SEQUENCE LISTING (105 sequences)

SEQ ID NO: 1 - protein, 113 aa, Artificial Sequence
  Synthesized Artificial Sequence
DIVMTQSPSSLTVTAGEKVTMSCKSSQSLLNSGNQKNYLTWYQQKPGQPPKLLIYWASTRESGVPDRFTGSGSGTDFTLTISSVQAEDLAVYYCQNDYSYPFTFGSGTKLEIK

SEQ ID NO: 2 - protein, 113 aa, Artificial Sequence
  Synthesized Artificial Sequence
DIVMTQSPDSLAVSLGERATINCKSSQSLLNSGNQKNYLTWYQQKPGQPPKLLIYWSSTRESGVPDRFSGSGSGTDFTLTISSLQAEDFAVYYCQNDYYYPLTFGGGTKVEIK

SEQ ID NO: 3 - protein, 113 aa, Artificial Sequence
  Synthesized Artificial Sequence
DIVMTQSPDSLAVSLGERATINCKSSQSLLNSGNQKNYLTWYQQKPGQPPKLLIYWASTRDSGVPDRFSGSGSGTDFTLTISSLQAEDVAVYYCQNDFIFPLTFGGGTKVEIK

SEQ ID NO: 4 - protein, 118 aa, Artificial Sequence
  Synthesized Artificial Sequence
QVQLQQPGAELVRPGASVKLSCKASGYTFTSYWINWVKQRPGQGLEWIGNIYPSDSYTNYNQKFKDKATLTVDKSSSTAYMQLSSPTSEDSAVYYCTRSWRGNSFDYWGQGTTLTVSS

SEQ ID NO: 5 - protein, 118 aa, Artificial Sequence
  Synthesized Artificial Sequence
EVQLVESGGGLVKPGGSLRLSCAASGFSFSNSAMSWVRQAPGKGLEWVSTISSGDSYTYYADSVKGRFTISRDNAKNSLYLQMNSLRAEDTAVYYCARQGYGNALDYWGQGTLVTVSS

SEQ ID NO: 6 - protein, 117 aa, Artificial Sequence
  Synthesized Artificial Sequence
QVQLQESGPGLVKPSETLSLTCTVSGFSLTRYGVSWIRQPPGKGLEWIGVIWGEGNTNYNPSLKSRVTISKDSSKSQVSLKLSSVTAADTAVYYCARVNFGNALDYWGQGTLVTVSS

SEQ ID NO: 7 - protein, 125 aa, Artificial Sequence
  Synthesized Artificial Sequence
QVQLVESGGGVVQPGRSLRLSCAASGFTFSTYAMNWVRQAPGKGLEWVARIRSKYNNYATYYADSVKDRFTISRDDSKNTLYLQMNSLRAEDTAVYYCARHGNFGNSYVSWAAYWGQGTLVTVSS

SEQ ID NO: 8 - protein, 15 aa, Artificial Sequence
  Synthesized Artificial Sequence
GGGGSGGGGSGGGGS

SEQ ID NO: 9 - protein, 109 aa, Artificial Sequence
  Synthesized Artificial Sequence
QTVVTQEPSLTVSPGGTVTLTCRSSTGAVTTSNYANWFQQKPGQAPRGLIGGTNKRAPGVPARFSGSLLGGKAALTLSGVQPEDEAEYYCALWYSNLWVFGGGTKVEIK

SEQ ID NO: 10 - protein, 125 aa, Artificial Sequence
  Synthesized Artificial Sequence
QVQLVESGGGVVQPGRSLRLSCAASGFTFSTYAMNWVRQAPGKGLEWVARIRSKYNNYATYYADSVKDRFTISRDDSKNTLYLQMNSLRAEDTAVYYCARHGNFGNSYVSWFAYWGQGTLVTVSS

SEQ ID NO: 11 - protein, 109 aa, Artificial Sequence
  Synthesized Artificial Sequence
QTVVTQEPSLTVSPGGTVTLTCRSSTGAVTTSNYANWVQQKPGQAPRGLIGGTNKRAPGVPARFSGSLLGGKAALTLSGVQPEDEAEYYCALWYSNLWVFGGGTKVEIK

SEQ ID NO: 12 - protein, 125 aa, Artificial Sequence
  Synthesized Artificial Sequence
QVQLVESGGGVVQPGRSLRLSCAASGFTFSTYAMNWVRQAPGKGLEWVGRIRSKYNNYATYYADSVKDRFTISRDDSKNTLYLQMNSLRGEDTAVYYCARHGNFGNSYVSWFAYWGQGTLVTVSS

SEQ ID NO: 13 - protein, 109 aa, Artificial Sequence
  Synthesized Artificial Sequence
QTVVTQEPSLTVSPGGTVTLTCRSSTGAVTTSNYANWVQQKPGQAPRGLIGGTNKRAPGVPARFSGSLLGGKAALTLSGVQPEDEAEYTCALWYSNLWVFGGGTKVEIK

SEQ ID NO: 14 - protein, 119 aa, Artificial Sequence
  Synthesized Artificial Sequence
DIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSS

SEQ ID NO: 15 - protein, 106 aa, Artificial Sequence
  Synthesized Artificial Sequence
DIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 16 - protein, 116 aa, Artificial Sequence
  Synthesized Artificial Sequence
EVQLVESGGGLVQPGKSLKLSCEASGFTFSGYGMHWVRQAPGRGLESVAYITSSSINIKYADAVKGRFTVSRDNAKNLLFLQMNILKSEDTAMYYCARFDWDKNYWGQGTMVTVSS

SEQ ID NO: 17 - protein, 107 aa, Artificial Sequence
  Synthesized Artificial Sequence
DIQMTQSPSSLPASLGDRVTINCQASQDISNYLNWYQQKPGKAPKLLIYYTNKLADGVPSRFSGSGSGRDSSFTISSLESEDIGSYYCQQYYNYPWTFGPGTKLEIK

SEQ ID NO: 18 - protein, 107 aa, Artificial Sequence [sequence omitted]

SEQ ID NO: 19 - protein, 98 aa, Artificial Sequence
  Synthesized Artificial Sequence
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKV

SEQ ID NO: 20 - protein, 209 aa, Artificial Sequence
  Synthesized Artificial Sequence
SVFLFPPKPKDTLMISRTPEVTCVVVDVSHEAPEVQFNWYVDGVEVHNAKTKPREEQFNSTFRVVSVLTVVHQDWLNGKEYKCKVSNKGLPAPIEKTISKTKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 21 - protein, 209 aa, Artificial Sequence
  Synthesized Artificial Sequence
SVFLFPPKPKDTLMISRTPEVTCVVVDVSHEAPEVQFNWYVDGVEVHNAKTKPREEQFNSTFRVVSVLTVVHQDWLNGKEYKCKVSNKGLPAPIEKTISKTKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 22 - protein, 209 aa, Artificial Sequence
  Synthesized Artificial Sequence
SVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYDTTPPVLDSDGSFFLYSDLTVDKSRWQQGNVFSCSVMHEALHNHYTQKSLSLSPGK

SEQ ID NO: 23 - protein, 209 aa, Artificial Sequence
  Synthesized Artificial Sequence
SVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCRVKGFYPSDIAVEWESNGQPENNYKTTPPVLKSDGSFFLAAKLTVDKSRWQQGNVFSCSVMHEALHNHYTQKSLSLSPGK

SEQ ID NO: 24 - protein, 209 aa, Artificial Sequence
  Synthesized Artificial Sequence
SVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 25 - protein, 209 aa, Artificial Sequence
  Synthesized Artificial Sequence
SVFLFPPKPKDTLMISRTPEVTCVVVFVSHEDPEVKFNWYVDGVEVHNAKTKPREEQYESTYRVVSVLTVLHQDWLNGKEYKCKVSNKALPAPEEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 26 - protein, 209 aa, Artificial Sequence
  Synthesized Artificial Sequence
SVFLFPPKPKDTLMISRTPEVTCVVVYVSHEDPEVKFNWYVDGVEVHNAKTKPREEQYDSLYRVVSVLTVLHQDWLNGKEYKCKVSNKALPAPEEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 27 - protein, 22 aa, Artificial Sequence
  Synthesized Artificial Sequence
EPKSCDKTHTCPPCPAPPVAGP

SEQ ID NO: 28 - protein, 17 aa, Artificial Sequence
  Synthesized Artificial Sequence
DKTHTCPPCPAPPVAGP

SEQ ID NO: 29 - protein, 10 aa, Artificial Sequence
  Synthesized Artificial Sequence
EPKSCDKTHT

SEQ ID NO: 30 - protein, 6 aa, Artificial Sequence
  Synthesized Artificial Sequence
GRGRGS

SEQ ID NO: 31 - protein, 16 aa, Artificial Sequence
  Synthesized Artificial Sequence
APAPAPAPAPAPAPAP

SEQ ID NO: 32 - protein, 5 aa, Artificial Sequence
  Synthesized Artificial Sequence
GGGGS

SEQ ID NO: 33 - protein, 220 aa, Artificial Sequence
  Synthesized Artificial Sequence
DIVMTQSPDSLAVSLGERATINCKSSQSLLNSGNQKNYLTWYQQKPGQPPKLLIYWASTRDSGVPDRFSGSGSGTDFTLTISSLQAEDVAVYYCQNDFIFPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 34 - protein, 447 aa, Artificial Sequence
  Synthesized Artificial Sequence
QVQLQESGPGLVKPSETLSLTCTVSGFSLTRYGVSWIRQPPGKGLEWIGVIWGEGNTNYNPSLKSRVTISKDSSKSQVSLKLSSVTAADTAVYYCARVNFGNALDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 35 - protein, 220 aa, Artificial Sequence
  Synthesized Artificial Sequence
DIVMTQSPSSLTVTAGEKVTMSCKSSQSLLNSGNQKNYLTWYQQKPGQPPKLLIYWASTRESGVPDRFTGSGSGTDFTLTISSVQAEDLAVYYCQNDYSYPFTFGSGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 36 - protein, 448 aa, Artificial Sequence
  Synthesized Artificial Sequence
QVQLQQPGAELVRPGASVKLSCKASGYTFTSYWINWVKQRPGQGLEWIGNIYPSDSYTNYNQKFKDKATLTVDKSSSTAYMQLSSPTSEDSAVYYCTRSWRGNSFDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 37 - protein, 216 aa, Artificial Sequence
  Synthesized Artificial Sequence
QTVVTQEPSLTVSPGGTVTLTCRSSTGAVTTSNYANWVQQKPGQAPRGLIGGTNKRAPGVPARFSGSLLGGKAALTLSGVQPEDEAEYYCALWYSNLWVFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 38 - protein, 454 aa, Artificial Sequence
  Synthesized Artificial Sequence
QVQLVESGGGVVQPGRSLRLSCAASGFTFSTYAMNWVRQAPGKGLEWVARIRSKYNNYATYYADSVKDRFTISRDDSKNTLYLQMNSLRAEDTAVYYCARHGNFGNSYVSWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPVAGPSVFLFPPKPKDTLMISRTPEVTCVVVDVSHEAPEVQFNWYVDGVEVHNAKTKPREEQFNSTFRVVSVLTVVHQDWLNGKEYKCKVSNKGLPAPIEKTISKTKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 39 - protein, 261 aa, Artificial Sequence
  Synthesized Artificial Sequence
MSTTTCQVVAFLLSILGLAGCIAATGMDMWSTQDLYDNPVTSVFQYEGLWRSCVRQSSGFTECRPYFTILGLPAMLQAVRALMIVGIVLGAIGLLVSIFALKCIRIGSMEDSAKANMTLTSGIMFIVSGLCAIAGVSVFANMLVTNFWMSTANMYTGMGGMVQTVQTRYTFGAALFVGWVAGGLTLIGGVMMCIACRGLAPEETNYKAVSYHASGHSVAYKPGGFKASTGFGSNTKNKKIYDGGARTEDEVQSYPSKHDYV

SEQ ID NO: 40 - protein, 261 aa, Artificial Sequence
  Synthesized Artificial Sequence
MAVTACQGLGFVVSLIGIAGIIAATCMDQWSTQDLYNNPVTAVFNYQGLWRSCVRESSGFTECRGYFTLLGLPAMLQAVRALMIVGIVLGAIGLLVSIFALKCIRIGSMEDSAKANMTLTSGIMFIVSGLCAIAGVSVFANMLVTNFWMSTANMYTGMGGMVQTVQTRYTFGAALFVGWVAGGLTLIGGVMMCIACRGLAPEETNYKAVSYHASGHSVAYKPGGFKASTGFGSNTKNKKIYDGGARTEDEVQSYPSKHDYV

SEQ ID NO: 41 - protein, 250 aa, Artificial Sequence
  Synthesized Artificial Sequence
MATTTCQVVGLLLSLLGLAGCIAATGMDMWSTQDLYDNPVTAVFQYEGLWRSCVQQSSGFTECRPYFTILGLPAMLQAVRALMIVGIVLGVIGILVSIFALKCIRIGSMDDSAKAKMTLTSGILFIISGICAIIGVSVFANMLVTNFWMSTANMYSGMGGMGGMVQTVQTRYTFGAALFVGWVAGGLTLIGGVMMCIACRGLTPDDSNFKAVSYHASGQNVAYRPGGFKASTGFGSNTRNKKIYDGGART

SEQ ID NO: 42 - protein, 200 aa, Artificial Sequence
  Synthesized Artificial Sequence
MATTTCQVVGLLLSLLGLAGCIAATGMDMWSTQDLYDNPVTAVFQYEGLWRSCVQQSSGFTECRPYFTILGLPAMLQAVRALMIVGIVLGVIGILVSIFALKCIRIGSMDDSAKAKMTLTSGILFIISGICAIIGVSVFANMLVTNFWMSTANMYSGMGGMGGMVQTVQTRYTFGAALFVGWVAGGLTLIGGVMMCIACR

SEQ ID NO: 43 - protein, 264 aa, Artificial Sequence
  Synthesized Artificial Sequence
MSVTACQGLGFVVSLIGFAGIIAATCMDQWSTQDLYNNPVTAVFNYQGLWRSCVRESSGFTECRGYFTLLGLPAMLQAVRALMIVGIVLGVIGILVSIFALKCIRIGSMDDSAKAKMTLTSGILFIISGICAIIGVSVFANMLVTNFWMSTANMYSGMGGMGGMVQTVQTRYTFGAALFVGWVAGGLTLIGGVMMCIACRGLTPDDSNFKAVSYHASGQNVAYRPGGFKASTGFGSNTRNKKIYDGGARTEDDEQSHPTKYDYV

SEQ ID NO: 44 - protein, 208 aa, Artificial Sequence
  Synthesized Artificial Sequence
MSVTACQGLGFVVSLIGFAGIIAATCMDQWSTQDLYNNPVTAVFNYQGLWRSCVRESSGFTECRGYFTLLGLPAMLQAVRALMIVGIVLGVIGILVSIFALKCIRIGSMDDSAKAKMTLTSGILFIISGICAIIGVSVFANMLVTNFWMSTANMYSGMGGMGGMVQTVQTRYTFGAALFVGWVAGGLTLIGGVMMCIACRGLTPDDSK

SEQ ID NO: 45 - protein, 104 aa, Artificial Sequence
  Synthesized Artificial Sequence
DGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMD

SEQ ID NO: 46 - protein, 160 aa, Artificial Sequence
  Synthesized Artificial Sequence
MRWNTFWGILCLSLLAVGTCQDDAENIEYKVSISGTSVELTCPLDSDENLKWEKNGQELPQKHDKHLVLQDFSEVEDSGYYVCYTPASNKNTYLYLKARVCEYCVEVDLTAVAIIIIVDICITLGLLMVIYYWSKNRKAKAKPVTRGTGAGSRPRGQNKE

SEQ ID NO: 47 - protein, 8 aa, Artificial Sequence
  Synthesized Artificial Seuqence
GYTFTSYW

SEQ ID NO: 48 - protein, 8 aa, Artificial Sequence
  Synthesized Artificial Sequence
IYPSDSYT

SEQ ID NO: 49 - protein, 11 aa, Artificial Sequence
  Synthesized Artificial Sequence
TRSWRGNSFDY

SEQ ID NO: 50 - protein, 12 aa, Artificial Sequence
  Synthesized Artificial Sequence
QSLLNSGNQKNY

SEQ ID NO: 51 - protein, 10 aa, Artificial Sequence
  Synthesized Artificial Sequence
WASTTTTTTT

SEQ ID NO: 52 - protein, 8 aa, Artificial Sequence
  Synthesized Artificial Sequence
QNDYSYPF

SEQ ID NO: 53 - protein, 8 aa, Artificial Sequence
  Synthesized Artificial Sequence
GFSFSNSA

SEQ ID NO: 54 - protein, 8 aa, Artificial Sequence
  Synthesized Artificial Sequence
ISSGDSYT

SEQ ID NO: 55 - protein, 11 aa, Artificial Sequence
  Synthesized Artificial Sequence
ARQGYGNALDY

SEQ ID NO: 56 - protein, 12 aa, Artificial Sequence
  Synthesized Artificial Sequence
QSLLNSGNQKNY

SEQ ID NO: 57 - protein, 8 aa, Artificial Sequence
  Synthesized Artificial Sequence
WSTTTTTT

SEQ ID NO: 58 - protein, 9 aa, Artificial Sequence
  Synthesized Artificial Sequence
QNDYYYPLT

SEQ ID NO: 59 - protein, 10 aa, Artificial Sequence
  Synthesized Artificial Sequence
GFSFSNSAMS

SEQ ID NO: 60 - protein, 10 aa, Artificial Sequence
  Synthesized Artificial Sequence
TISSGDSYTY

SEQ ID NO: 61 - protein, 9 aa, Artificial Sequence
  Synthesized Artificial Sequence
QGYGNALDY

SEQ ID NO: 62 - protein, 17 aa, Artificial Sequence
  Synthesized Artificial Sequence
KSSQSLLNSGNQKNYLT

SEQ ID NO: 63 - protein, 7 aa, Artificial Sequence
  Synthesized Artificial Sequence
WSSTRES

SEQ ID NO: 64 - protein, 5 aa, Artificial Sequence
  Synthesized Artificial Sequence
NSAMS

SEQ ID NO: 65 - protein, 17 aa, Artificial Sequence
  Synthesized Artificial Sequence
TISSGDSYTYYADSVKG

SEQ ID NO: 66 - protein, 8 aa, Artificial Sequence
  Synthesized Artificial Sequence
GFSLTRYG

SEQ ID NO: 67 - protein, 7 aa, Artificial Sequence
  Synthesized Artificial Sequence
IWGEGNT

SEQ ID NO: 68 - protein, 11 aa, Artificial Sequence
  Synthesized Artificial Sequence
ARVNFGNALDY

SEQ ID NO: 69 - protein, 12 aa, Artificial Sequence
  Synthesized Artificial Sequence
QSLLNSGNQKNY

SEQ ID NO: 70 - protein, 8 aa, Artificial Sequence
  Synthesized Artificial Sequence
WATTTTTT

SEQ ID NO: 71 - protein, 9 aa, Artificial Sequence
  Synthesized Artificial Sequence
QNDFIFPLT

SEQ ID NO: 72 - protein, 10 aa, Artificial Sequence
  Synthesized Artificial Sequence
GFSLTRYGVS

SEQ ID NO: 73 - protein, 9 aa, Artificial Sequence
  Synthesized Artificial Sequence
VIWGEGNTN

SEQ ID NO: 74 - protein, 9 aa, Artificial Sequence
  Synthesized Artificial Sequence
VNFGNALDY

SEQ ID NO: 75 - protein, 17 aa, Artificial Sequence
  Synthesized Artificial Sequence
KSSQSLLNSGNQKNYLT

SEQ ID NO: 76 - protein, 7 aa, Artificial Sequence
  Synthesized Artificial Sequence
WASTRDS

SEQ ID NO: 77 - protein, 5 aa, Artificial Sequence
  Synthesized Artificial Sequence
RYGVS

SEQ ID NO: 78 - protein, 16 aa, Artificial Sequence
  Synthesized Artificial Sequence
VIWGEGNTNYNPSLKS

SEQ ID NO: 79 - protein, 5 aa, Artificial Sequence
  Synthesized Artificial Sequence
TYAMN

SEQ ID NO: 80 - protein, 19 aa, Artificial Sequence
  Synthesized Artificial Sequence
RIRSKYNNYATYYADSVKD

SEQ ID NO: 81 - protein, 14 aa, Artificial Sequence
  Synthesized Artificial Sequence
HGNFGNSYVSWAAY

SEQ ID NO: 82 - protein, 14 aa, Artificial Sequence
  Synthesized Artificial Sequence
RSSTGAVTTSNYAN

SEQ ID NO: 83 - protein, 7 aa, Artificial Sequence
  Synthesized Artificial Sequence
GTNKRAP

SEQ ID NO: 84 - protein, 9 aa, Artificial Sequence
  Synthesized Artificial Sequence
ALWYSNLWV

SEQ ID NO: 85 - protein, 14 aa, Artificial Sequence
  Synthesized Artificial Sequence
HGNFGNSYVSWFAY

SEQ ID NO: 86 - protein, 5 aa, Artificial Sequence
  Synthesized Artificial Sequence
RYTMH

SEQ ID NO: 87 - protein, 17 aa, Artificial Sequence
  Synthesized Artificial Sequence
YINPSRGYTNYNQKFKD

SEQ ID NO: 88 - protein, 10 aa, Artificial Sequence
  Synthesized Artificial Sequence
YYDDHYCLDY

SEQ ID NO: 89 - protein, 10 aa, Artificial Sequence
  Synthesized Artificial Sequence
RASSSVSYMN

SEQ ID NO: 90 - protein, 7 aa, Artificial Sequence
  Synthesized Artificial Sequence
DTSKVAS

SEQ ID NO: 91 - protein, 9 aa, Artificial Sequence
  Synthesized Artificial Sequence
QQWSSNPLT

SEQ ID NO: 92 - protein, 5 aa, Artificial Sequence
  Synthesized Artificial Sequence
GYGMH

SEQ ID NO: 93 - protein, 17 aa, Artificial Sequence
  Synthesized Artificial Sequence
YITSSSINIKYADAVKG

SEQ ID NO: 94 - protein, 7 aa, Artificial Sequence
  Synthesized Artificial Sequence
FDWDKNY

SEQ ID NO: 95 - protein, 11 aa, Artificial Sequence
  Synthesized Artificial Sequence
QASQDISNYLN

SEQ ID NO: 96 - protein, 7 aa, Artificial Sequence
  Synthesized Artificial Sequence
YTNKLAD

SEQ ID NO: 97 - protein, 9 aa, Artificial Sequence
  Synthesized Artificial Sequence
QQYYNYPWT

SEQ ID NO: 98 - protein, 106 aa, Artificial Sequence
  Synthesized Artificial Sequence
GQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 99 - protein, 106 aa, Artificial Sequence
  Synthesized Artificial Sequence
GQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 100 - protein, 106 aa, Artificial Sequence
  Synthesized Artificial Sequence
GQPKAAPSVTLFPPSSEELQANKATLVCLISDFYPGAVTVAWKADSSPVKAGVETTTPSKQSNNKYAASSYLSLTPEQWKSHKSYSCQVTHEGSTVEKTVAPTECS

SEQ ID NO: 101 - protein, 98 aa, Artificial Sequence
  Synthesized Artificial Sequence
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTV

SEQ ID NO: 102 - protein, 98 aa, Artificial Sequence
  Synthesized Artificial Sequence
ASTKGPSVFPLAPCSRSTSGGTAALGCLVKDYFPEPVTVSWNSGALTSGVHTFPAVLQSSGLYSLSSVVTVPSSSLGTQTYTCNVNHKPSNTKVDKRV

SEQ ID NO: 103 - protein, 98 aa, Artificial Sequence
  Synthesized Artificial Sequence
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRV

SEQ ID NO: 104 - protein, 220 aa, Artificial Sequence
  Synthesized Artificial Sequence
DIVMTQSPDSLAVSLGERATINCKSSQSLLNSGNQKNYLTWYQQKPGQPPKLLIYWSSTRESGVPDRFSGSGSGTDFTLTISSLQAEDFAVYYCQNDYYYPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 105 - protein, 448 aa, Artificial Sequence
  Synthesized Artificial Sequence
EVQLVESGGGLVKPGGSLRLSCAASGFSFSNSAMSWVRQAPGKGLEWVSTISSGDSYTYYADSVKGRFTISRDNAKNSLYLQMNSLRAEDTAVYYCARQGYGNALDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

(19) United States
(12) Patent Application Publication
(10) Pub. No.: US 2023/0192903 A1
(43) Pub. Date: Jun. 22, 2023

(54) BISPECIFIC ANTIBODY AGAINST CLDN18.2 AND CD3

(71) Applicant: Wuhan YZY Biopharma Co., Ltd., Wuhan, Hubei (CN)

(72) Inventors: Hao YANG, Wuhan, Hubei (CN); Lijuan FANG, Wuhan, Hubei (CN); Shan HUA, Wuhan, Hubei (CN); Jing ZHANG, Wuhan, Hubei (CN); Pengfei ZHOU, Wuhan, Hubei (CN)

(21) Appl. No.: 18/000,174

(22) PCT Filed: May 29, 2020
(86) PCT No.: PCT/CN2020/093473

§ 371 Date: Nov. 29, 2022

(51) Int. Cl. C07K 16/46; A61P 35/00

(52) U.S. Cl. CPC C07K 16/468; A61P 35/00; C07K 2317/31; C07K 2317/565; C07K 2317/622; C07K 2317/522

(57) ABSTRACT

The present invention relates to a bispecific antibody against CLDN18.2 and CD3. The bispecific antibody effectively binds to CLDN18.2 and CD3 antigens.

### TECHNICAL FIELD

The present invention belongs to the field of antibody. In particular, the present invention relates to a bispecific antibody against CLDN18.2 and CD3, an anti-CD3 single-chain antibody and use thereof.

### BACKGROUND

Gastric cancer occurs regionally, with the morbidity and mortality of gastric cancer in china being among the top three. It is a characteristic cancer with high incidence mainly caused by dietary habits, which seriously harms the life health of people in China. In November 2019, the National Cancer Center released the latest national cancer statistics, i.e., the morbidity and mortality of malignant tumors in China in 2015. Among them, the morbidity and mortality of gastric cancer ranked the third in China, with 503,000 cases of gastric cancer (281,000 cases in males and 122,000 cases in females) and 291,000 deaths (201,000 cases in males and 90,000 cases in females). At present, the pathogenesis genes in gastric cancer are not very clear, and researches show that the occurrence of gastric cancer is related to Helicobacter pylori and EBV virus infection, and the gastric cancer has the characteristic of family aggregation. In the early stage of gastric cancer, endoscopic and surgical treatment are often beneficial to the resection of gastric cancer; however, gastric cancer often faces recurrence risk. Therefore, additional lymphatic resection, radiotherapy, postoperative chemotherapy, and the like are required according to the actual clinical condition. For unresectable locally-advanced gastric cancer, and recurrent and metastatic gastric cancer, conservative treatments are generally used, including systemic therapy, clinical trial and supportive care. In fact, most gastric cancers are found in an advanced stage, and the probability of surgical treatment is relatively low.

In the therapy of gastric cancer, HER2 status of patient is generally first analyzed by immunohistochemistry (IHC) and fluorescence in situ hybridization (FISH) to determine whether Trastuzumab is used. The currently recognized first-line therapeutic drugs are combination therapies of two or three chemical drugs, such as a combination of fluoropyrimidine-based and platinum-based drugs, paclitaxel and docetaxel in combination with platinum-based drugs, and the like; and for the elderly patients, second-line and later-line therapeutic drugs are generally preferred. The targeted therapeutic drugs for gastric cancer include HER2 targeted antibody Trastuzumab, VEGFR targeted antibody Ramucirumab, PD-L1 targeted antibody Pembrolizumab and PD-1 targeted antibody Nivolumab. Among them, Trastuzumab was approved by FDA for the treatment of gastric cancer in 2010; however, according to the phase III (ToGA) trial of Trastuzumab, only about 22% of patients with gastric cancer were HER2 positive, and the population with high expression (IHC2+ and FISH+ or IHC3+) significantly benefiting from Trastuzumab only accounted for about 12% of patients with gastric cancer, so Trastuzumab benefited only a limited number of patients. Ramucirumab was approved by FDA for the treatment of gastric cancer in 2014; according to the published result analysis of the phase III (Rainbow) trial, the antibody Ramucirumab in combination with paclitaxel extended median disease-free survival from 2.9 months to 4.4 months and median overall survival in non-Asian patients from 5.9 months to 8.5 months compared to paclitaxel in combination with placebo, but had no significant advantage in the indicator of extending overall survival in Asian patients. Immune checkpoint antibodies Pembrolizumab and Nivolumab have also been approved in recent years for use in the second-line and later-line therapy for gastric cancer. However, the existing targeted therapeutic drugs for gastric cancer are still very lacking.

The Claudin (CLDN) family was first discovered in 1998, and most of the family member proteins are four-transmembrane proteins, comprising two extracellular domains. The CLDN family protein is an important component of cellular tight junction, and closely links adjacent cells together with other tight junction protein families such as Occludin proteins and junction adhesion molecule (JAM) proteins through the interaction with cytoskeleton protein, thereby controlling the passing of substances among the cells. Different proteins in the CLDN family are expressed on different tissues, with CLDN18 being specifically expressed in gastric tissue. CLDN18 has two subtypes of CLDN18.1 and CLDN18.2; the CLDN18.2 protein in gastric tissue is beneficial to control the penetration of intercellular H+ to avoid the resulting immune activation and gastritis, and CLDN18.2 is exposed due to the disappearance of tight junctions between cells in the process of gastric tumorigenesis, so that CLDN18.2 can become one of the targets of gastric cancer-specific drug development. CLDN18.2 is detectably positive in 70-90% of patients with gastric cancer, the expression of CLDN18.2 in other tumors may also be induced, including about 50% of pancreatic cancer, 30% of esophageal cancer, and about 25% of non-small cell lung cancer, and expression of CLDN18.2 is still detectable in metastases from these tumors. Therefore, CLDN18.2 can also become one of targeted drug development targets for pancreatic cancer, esophageal cancer and non-small cell lung cancer. CLDN18.2 has great potential as a targeted target for gastric cancer.

### SUMMARY

A bispecific antibody is an antibody molecule comprising two specific antigen-binding sites, wherein an immune cell-recruiting antibody can simultaneously bind to a tumor cell specific antigen and an immune cell surface antigen, and can then effectively bridge tumor cells and immune cells, thereby promoting the killing effect of immune cells on tumor cells; wherein a T cell-recruiting bispecific antibody has both a binding domain of the T cell surface molecule CD3 and a binding domain of a target cell surface antigen, thereby promoting the specific killing effect of T cells on target cells. Such a bispecific antibody can activate CD3+T cells without co-stimulatory molecules and MHC-antigen presentation, and can play a role in killing. The CLDN18.2 on the surface of the tumor cell provides a target for a bispecific antibody targeting CLDN18.2 and CD3, then promoting the killing effect of CD3+T cells on CLDN18.2 positive tumor cells in gastric cancer, pancreatic cancer and other types of tumors. The present application provides a novel bispecific antibody targeting the CLDN18.2 antigen and the CD3 antigen.

Specifically, the present invention relates to the following aspects:

1. A bispecific antibody, comprising an antigen-binding domain specifically binding to CD3 and an antigen-binding domain specifically binding to CLDN18.2, wherein the antigen-binding domain specifically binding to CD3 is selected from the group consisting of:
1). an antigen-binding domain specifically binding to CD3 comprising the following CDRs or variants thereof:
(i) CDRH1, CDRH2 and CDRH3 comprised in a heavy chain variable region set forth in any one of SEQ ID NO: 7, 10 or 12, and
(ii) CDRL1, CDRL2 and CDRL3 comprised in a light chain variable region set forth in any one of SEQ ID NO: 9, 11 or 13,
preferably, the CDRH1 has a sequence set forth in SEQ ID NO: 79, the CDRH2 has a sequence set forth in SEQ ID NO: 80, the CDRH3 has a sequence set forth in SEQ ID NO: 81 or 85, the CDRL1 has a sequence set forth in SEQ ID NO: 82, the CDRL2 has a sequence set forth in SEQ ID NO: 83, and the CDRL3 has a sequence set forth in SEQ ID NO: 84;
2). an antigen-binding domain specifically binding to CD3 comprising the following CDRs or variants thereof:
(i) CDRH1, CDRH2 and CDRH3 comprised in a heavy chain variable region set forth in SEQ ID NO: 14, and
(ii) CDRL1, CDRL2 and CDRL3 comprised in a light chain variable region set forth in SEQ ID NO: 15,
preferably, the CDRH1 has a sequence set forth in SEQ ID NO: 86, the CDRH2 has a sequence set forth in SEQ ID NO: 87, the CDRH3 has a sequence set forth in SEQ ID NO: 88, the CDRL1 has a sequence set forth in SEQ ID NO: 89, the CDRL2 has a sequence set forth in SEQ ID NO: 90, and the CDRL3 has a sequence set forth in SEQ ID NO: 91; or
3). an antigen-binding domain specifically binding to CD3 comprising the following CDRs or variants thereof:
(i) CDRH1, CDRH2 and CDRH3 comprised in a heavy chain variable region set forth in SEQ ID NO: 16, and
(ii) CDRL1, CDRL2 and CDRL3 comprised in a light chain variable region set forth in SEQ ID NO: 17,
preferably, the CDRH1 has a sequence set forth in SEQ ID NO: 92, the CDRH2 has a sequence set forth in SEQ ID NO: 93, the CDRH3 has a sequence set forth in SEQ ID NO: 94, the CDRL1 has a sequence set forth in SEQ ID NO: 95, the CDRL2 has a sequence set forth in SEQ ID NO: 96, and the CDRL3 has a sequence set forth in SEQ ID NO: 97; and
wherein the antigen-binding domain specifically binding to CLDN18.2 is selected from the group consisting of:
1) an antigen-binding domain specifically binding to CLDN18.2 comprising the following CDRs or variants thereof:
(i) CDRH1, CDRH2 and CDRH3 comprised in a heavy chain variable region set forth in SEQ ID NO: 4, and
(ii) CDRL1, CDRL2 and CDRL3 comprised in a light chain variable region set forth in SEQ ID NO: 1,
preferably, the CDRH1 has a sequence set forth in SEQ ID NO: 47, the CDRH2 has a sequence set forth in SEQ ID NO: 48, the CDRH3 has a sequence set forth in SEQ ID NO: 49, the CDRL1 has a sequence set forth in SEQ ID NO: 50, the CDRL2 has a sequence set forth in SEQ ID NO: 51, and the CDRL3 has a sequence set forth in SEQ ID NO: 52;
2) an antigen-binding domain specifically binding to CLDN18.2 comprising the following CDRs or variants thereof:
(i) CDRH1, CDRH2 and CDRH3 comprised in a heavy chain variable region set forth in SEQ ID NO: 5, and
(ii) CDRL1, CDRL2 and CDRL3 comprised in a light chain variable region set forth in SEQ ID NO: 2,
preferably, the CDRH1 is selected from SEQ ID NOs: 53, 59 and 64, the CDRH2 is selected from SEQ ID NOs: 54, 60 and 65, the CDRH3 is selected from SEQ ID NOs: 55 and 61, the CDRL1 is selected from SEQ ID NOs: 56 and 62, the CDRL2 is selected from SEQ ID NOs: 57 and 63, and the CDRL3 is set forth in SEQ ID NO: 58;
more preferably, the CDRH1 has a sequence set forth in SEQ ID NO: 53, the CDRH2 has a sequence set forth in SEQ ID NO: 54, the CDRH3 has a sequence set forth in SEQ ID NO: 55, the CDRL1 has a sequence set forth in SEQ ID NO: 56, the CDRL2 has a sequence set forth in SEQ ID NO: 57, and the CDRL3 has a sequence set forth in SEQ ID NO: 58;
more preferably, the CDRH1 has a sequence set forth in SEQ ID NO: 59, the CDRH2 has a sequence set forth in SEQ ID NO: 60, the CDRH3 has a sequence set forth in SEQ ID NO: 61, the CDRL1 has a sequence set forth in SEQ ID NO: 62, the CDRL2 has a sequence set forth in SEQ ID NO: 63, and the CDRL3 has a sequence set forth in SEQ ID NO: 58;
more preferably, the CDRH1 has a sequence set forth in SEQ ID NO: 64, the CDRH2 has a sequence set forth in SEQ ID NO: 65, the CDRH3 has a sequence set forth in SEQ ID NO: 61, the CDRL1 has a sequence set forth in SEQ ID NO: 62, the CDRL2 has a sequence set forth in SEQ ID NO: 63, and the CDRL3 has a sequence set forth in SEQ ID NO: 58; or
3) an antigen-binding domain specifically binding to CLDN18.2 comprising the following CDRs or variants thereof:
(i) CDRH1, CDRH2 and CDRH3 comprised in a heavy chain variable region set forth in SEQ ID NO: 6, and
(ii) CDRL1, CDRL2 and CDRL3 comprised in a light chain variable region set forth in SEQ ID NO: 3,
preferably, the CDRH1 is selected from SEQ ID NOs: 66, 72 and 77, the CDRH2 is selected from SEQ ID NOs: 67, 73 and 78, the CDRH3 is selected from SEQ ID NOs: 68 and 74, the CDRL1 is selected from SEQ ID NOs: 69 and 75, the CDRL2 is selected from SEQ ID NOs: 70 and 76, and the CDRL3 is set forth in SEQ ID NO: 71;
more preferably, the CDRH1 has a sequence set forth in SEQ ID NO: 66, the CDRH2 has a sequence set forth in SEQ ID NO: 67, the CDRH3 has a sequence set forth in SEQ ID NO: 68, the CDRL1 has a sequence set forth in SEQ ID NO: 69, the CDRL2 has a sequence set forth in SEQ ID NO: 70, and the CDRL3 has a sequence set forth in SEQ ID NO: 71;
more preferably, the CDRH1 has a sequence set forth in SEQ ID NO: 72, the CDRH2 has a sequence set forth in SEQ ID NO: 73, the CDRH3 has a sequence set forth in SEQ ID NO: 74, the CDRL1 has a sequence set forth in SEQ ID NO: 75, the CDRL2 has a sequence set forth in SEQ ID NO: 76, and the CDRL3 has a sequence set forth in SEQ ID NO: 71;
more preferably, the CDRH1 has a sequence set forth in SEQ ID NO: 77, the CDRH2 has a sequence set forth in SEQ ID NO: 78, the CDRH3 has a sequence set forth in SEQ ID NO: 74, the CDRL1 has a sequence set forth in SEQ ID NO: 75, the CDRL2 has a sequence set forth in SEQ ID NO: 76, and the CDRL3 has a sequence set forth in SEQ ID NO: 71,
wherein the variants differ from the CDRs by 3, 2 or 1 amino acids or have at least 80%, 85%, 90%, 91%, 92%, 93%, 94%, 95%, 96%, 97%, 98% or 99% identity thereto.
2. The bispecific antibody according to item 1, comprising an antigen-binding domain specifically binding to CD3 and an antigen-binding domain specifically binding to CLDN18.2,
wherein the antigen-binding domain specifically binding to CLDN18.2 comprises the following CDRs (or variants thereof) or variable regions (or variants thereof):
(i) CDRH1, CDRH2 and CDRH3 comprised in a heavy chain variable region set forth in SEQ ID NO: 4, or a heavy chain variable region set forth in SEQ ID NO: 4, and
(ii) CDRL1, CDRL2 and CDRL3 comprised in a light chain variable region set forth in SEQ ID NO: 1, or
a light chain variable region set forth in SEQ ID NO: 1,
preferably, the CDRH1 has a sequence set forth in SEQ ID NO: 47, the CDRH2 has a sequence set forth in SEQ ID NO: 48, the CDRH3 has a sequence set forth in SEQ ID NO: 49, the CDRL1 has a sequence set forth in SEQ ID NO: 50, the CDRL2 has a sequence set forth in SEQ ID NO: 51, and the CDRL3 has a sequence set forth in SEQ ID NO: 52; and
wherein the antigen-binding domain specifically binding to CD3 is selected from the group consisting of:
1). an antigen-binding domain specifically binding to CD3 comprising the following CDRs (or variants thereof) or variable regions (or variants thereof):
(i) CDRH1, CDRH2 and CDRH3 comprised in a heavy chain variable region set forth in SEQ ID NO: 7, or a heavy chain variable region set forth in SEQ ID NO: 7, and
(ii) CDRL1, CDRL2 and CDRL3 comprised in a light chain variable region set forth in SEQ ID NO: 9, or
a light chain variable region set forth in SEQ ID NO: 9,
preferably, the CDRH1 has a sequence set forth in SEQ ID NO: 79, the CDRH2 has a sequence set forth in SEQ ID NO: 80, the CDRH3 has a sequence set forth in SEQ ID NO: 81, the CDRL1 has a sequence set forth in SEQ ID NO: 82, the CDRL2 has a sequence set forth in SEQ ID NO: 83, and the CDRL3 has a sequence set forth in SEQ ID NO: 84;
2). an antigen-binding domain specifically binding to CD3 comprising the following CDRs (or variants thereof) or variable regions (or variants thereof):
(i) CDRH1, CDRH2 and CDRH3 comprised in a heavy chain variable region set forth in SEQ ID NO: 10, or
a heavy chain variable region set forth in SEQ ID NO: 10, and
(ii) CDRL1, CDRL2 and CDRL3 comprised in a light chain variable region set forth in SEQ ID NO: 11, or
a light chain variable region set forth in SEQ ID NO: 11,
preferably, the CDRH1 has a sequence set forth in SEQ ID NO: 79, the CDRH2 has a sequence set forth in SEQ ID NO: 80, the CDRH3 has a sequence set forth in SEQ ID NO: 85, the CDRL1 has a sequence set forth in SEQ ID NO: 82, the CDRL2 has a sequence set forth in SEQ ID NO: 83, and the CDRL3 has a sequence set forth in SEQ ID NO: 84;
3). an antigen-binding domain specifically binding to CD3 comprising the following CDRs (or variants thereof) or variable regions (or variants thereof):
(i) CDRH1, CDRH2 and CDRH3 comprised in a heavy chain variable region set forth in SEQ ID NO: 12, or
a heavy chain variable region set forth in SEQ ID NO: 12, and
(ii) CDRL1, CDRL2 and CDRL3 comprised in a light chain variable region set forth in SEQ ID NO: 13, or
a light chain variable region set forth in SEQ ID NO: 13,
preferably, the CDRH1 has a sequence set forth in SEQ ID NO: 79, the CDRH2 has a sequence set forth in SEQ ID NO: 80, the CDRH3 has a sequence set forth in SEQ ID NO: 85, the CDRL1 has a sequence set forth in SEQ ID NO: 82, the CDRL2 has a sequence set forth in SEQ ID NO: 83, and the CDRL3 has a sequence set forth in SEQ ID NO: 84;
4). an antigen-binding domain specifically binding to CD3 comprising the following CDRs (or variants thereof) or variable regions (or variants thereof):
(i) CDRH1, CDRH2 and CDRH3 comprised in a heavy chain variable region set forth in SEQ ID NO: 14, or
a heavy chain variable region set forth in SEQ ID NO: 14, and
(ii) CDRL1, CDRL2 and CDRL3 comprised in a light chain variable region set forth in SEQ ID NO: 15, or
a light chain variable region set forth in SEQ ID NO: 15,
preferably, the CDRH1 has a sequence set forth in SEQ ID NO: 86, the CDRH2 has a sequence set forth in SEQ ID NO: 87, the CDRH3 has a sequence set forth in SEQ ID NO: 88, the CDRL1 has a sequence set forth in SEQ ID NO: 89, the CDRL2 has a sequence set forth in SEQ ID NO: 90, and the CDRL3 has a sequence set forth in SEQ ID NO: 91; or
5). an antigen-binding domain specifically binding to CD3 comprising the following CDRs (or variants thereof) or variable regions (or variants thereof):
(i) CDRH1, CDRH2 and CDRH3 comprised in a heavy chain variable region set forth in SEQ ID NO: 16, or
a heavy chain variable region set forth in SEQ ID NO: 16, and
(ii) CDRL1, CDRL2 and CDRL3 comprised in a light chain variable region set forth in SEQ ID NO: 17, or
a light chain variable region set forth in SEQ ID NO: 17,
preferably, the CDRH1 has a sequence set forth in SEQ ID NO: 92, the CDRH2 has a sequence set forth in SEQ ID NO: 93, the CDRH3 has a sequence set forth in SEQ ID NO: 94, the CDRL1 has a sequence set forth in SEQ ID NO: 95, the CDRL2 has a sequence set forth in SEQ ID NO: 96, and the CDRL3 has a sequence set forth in SEQ ID NO: 97,
wherein the variants differ from the CDRs or the variable regions by 3, 2 or 1 amino acids or have at least 80%, 85%, 90%, 91%, 92%, 93%, 94%, 95%, 96%, 97%, 98% or 99% identity thereto.
3. The bispecific antibody according to item 1, comprising an antigen-binding domain specifically binding to CD3 and an antigen-binding domain specifically binding to CLDN18.2,
wherein the antigen-binding domain specifically binding to CLDN18.2 comprises the following CDRs (or variants thereof) or variable regions (or variants thereof):
(i) CDRH1, CDRH2 and CDRH3 comprised in a heavy chain variable region set forth in SEQ ID NO: 5, or
a heavy chain variable region set forth in SEQ ID NO: 5, and
(ii) CDRL1, CDRL2 and CDRL3 comprised in a light chain variable region set forth in SEQ ID NO: 2, or
a light chain variable region set forth in SEQ ID NO: 2,
preferably, the CDRH1 is selected from SEQ ID NOs: 53, 59 and 64, the CDRH2 is selected from SEQ ID NOs: 54, 60 and 65, the CDRH3 is selected from SEQ ID NOs: 55 and 61, the CDRL1 is selected from SEQ ID NOs: 56 and 62, the CDRL2 is selected from SEQ ID NOs: 57 and 63, and the CDRL3 is set forth in SEQ ID NO: 58;
more preferably, the CDRH1 has a sequence set forth in SEQ ID NO: 53, the CDRH2 has a sequence set forth in SEQ ID NO: 54, the CDRH3 has a sequence set forth in SEQ ID NO: 55, the CDRL1 has a sequence set forth in SEQ ID NO: 56, the CDRL2 has a sequence set forth in SEQ ID NO: 57, and the CDRL3 has a sequence set forth in SEQ ID NO: 58; more preferably, the CDRH1 has a sequence set forth in SEQ ID NO: 59, the CDRH2 has a sequence set forth in SEQ ID NO: 60, the CDRH3 has a sequence set forth in SEQ ID NO: 61, the CDRL1 has a sequence set forth in SEQ ID NO: 62, the CDRL2 has a sequence set forth in SEQ ID NO: 63, and the CDRL3 has a sequence set forth in SEQ ID NO: 58;
more preferably, the CDRH1 has a sequence set forth in SEQ ID NO: 64, the CDRH2 has a sequence set forth in SEQ ID NO: 65, the CDRH3 has a sequence set forth in SEQ ID NO: 61, the CDRL1 has a sequence set forth in SEQ ID NO: 62, the CDRL2 has a sequence set forth in SEQ ID NO: 63, and the CDRL3 has a sequence set forth in SEQ ID NO: 58; and
wherein the antigen-binding domain specifically binding to CD3 is selected from the group consisting of:
1). an antigen-binding domain specifically binding to CD3 comprising the following CDRs (or variants thereof) or variable regions (or variants thereof):
(i) CDRH1, CDRH2 and CDRH3 comprised in a heavy chain variable region set forth in SEQ ID NO: 7, or
a heavy chain variable region set forth in SEQ ID NO: 7, and
(ii) CDRL1, CDRL2 and CDRL3 comprised in a light chain variable region set forth in SEQ ID NO: 9, or
a light chain variable region set forth in SEQ ID NO: 9,
preferably, the CDRH1 has a sequence set forth in SEQ ID NO: 79, the CDRH2 has a sequence set forth in SEQ ID NO: 80, the CDRH3 has a sequence set forth in SEQ ID NO: 81, the CDRL1 has a sequence set forth in SEQ ID NO: 82, the CDRL2 has a sequence set forth in SEQ ID NO: 83, and the CDRL3 has a sequence set forth in SEQ ID NO: 84;
2). an antigen-binding domain specifically binding to CD3 comprising the following CDRs (or variants thereof) or variable regions (or variants thereof):
(i) CDRH1, CDRH2 and CDRH3 comprised in a heavy chain variable region set forth in SEQ ID NO: 10, or
a heavy chain variable region set forth in SEQ ID NO: 10, and
(ii) CDRL1, CDRL2 and CDRL3 comprised in a light chain variable region set forth in SEQ ID NO: 11, or
a light chain variable region set forth in SEQ ID NO: 11,
preferably, the CDRH1 has a sequence set forth in SEQ ID NO: 79, the CDRH2 has a sequence set forth in SEQ ID NO: 80, the CDRH3 has a sequence set forth in SEQ ID NO: 85, the CDRL1 has a sequence set forth in SEQ ID NO: 82, the CDRL2 has a sequence set forth in SEQ ID NO: 83, and the CDRL3 has a sequence set forth in SEQ ID NO: 84;
3). an antigen-binding domain specifically binding to CD3 comprising the following CDRs (or variants thereof) or variable regions (or variants thereof):
(i) CDRH1, CDRH2 and CDRH3 comprised in a heavy chain variable region set forth in SEQ ID NO: 12, or
a heavy chain variable region set forth in SEQ ID NO: 12, and
(ii) CDRL1, CDRL2 and CDRL3 comprised in a light chain variable region set forth in SEQ ID NO: 13, or
a light chain variable region set forth in SEQ ID NO: 13,
preferably, the CDRH1 has a sequence set forth in SEQ ID NO: 79, the CDRH2 has a sequence set forth in SEQ ID NO: 80, the CDRH3 has a sequence set forth in SEQ ID NO: 85, the CDRL1 has a sequence set forth in SEQ ID NO: 82, the CDRL2 has a sequence set forth in SEQ ID NO: 83, and the CDRL3 has a sequence set forth in SEQ ID NO: 84;
4). an antigen-binding domain specifically binding to CD3 comprising the following CDRs (or variants thereof) or variable regions (or variants thereof):
(i) CDRH1, CDRH2 and CDRH3 comprised in a heavy chain variable region set forth in SEQ ID NO: 14, or
a heavy chain variable region set forth in SEQ ID NO: 14, and
(ii) CDRL1, CDRL2 and CDRL3 comprised in a light chain variable region set forth in SEQ ID NO: 15, or
a light chain variable region set forth in SEQ ID NO: 15,
preferably, the CDRH1 has a sequence set forth in SEQ ID NO: 86, the CDRH2 has a sequence set forth in SEQ ID NO: 87, the CDRH3 has a sequence set forth in SEQ ID NO: 88, the CDRL1 has a sequence set forth in SEQ ID NO: 89, the CDRL2 has a sequence set forth in SEQ ID NO: 90, and the CDRL3 has a sequence set forth in SEQ ID NO: 91; or
5). an antigen-binding domain specifically binding to CD3 comprising the following CDRs (or variants thereof) or variable regions (or variants thereof):
(i) CDRH1, CDRH2 and CDRH3 comprised in a heavy chain variable region set forth in SEQ ID NO: 16, or
a heavy chain variable region set forth in SEQ ID NO: 16, and
(ii) CDRL1, CDRL2 and CDRL3 comprised in a light chain variable region set forth in SEQ ID NO: 17, or
a light chain variable region set forth in SEQ ID NO: 17,
preferably, the CDRH1 has a sequence set forth in SEQ ID NO: 92, the CDRH2 has a sequence set forth in SEQ ID NO: 93, the CDRH3 has a sequence set forth in SEQ ID NO: 94, the CDRL1 has a sequence set forth in SEQ ID NO: 95, the CDRL2 has a sequence set forth in SEQ ID NO: 96, and the CDRL3 has a sequence set forth in SEQ ID NO: 97,
wherein the variants differ from the CDRs or the variable regions by 3, 2 or 1 amino acids or have at least 80%, 85%, 90%, 91%, 92%, 93%, 94%, 95%, 96%, 97%, 98% or 99% identity thereto.
4. The bispecific antibody according to item 1, comprising an antigen-binding domain specifically binding to CD3 and an antigen-binding domain specifically binding to CLDN18.2,
wherein the antigen-binding domain specifically binding to CLDN18.2 comprises the following CDRs (or variants thereof) or variable regions (or variants thereof):
(i) CDRH1, CDRH2 and CDRH3 comprised in a heavy chain variable region set forth in SEQ ID NO: 6, or
a heavy chain variable region set forth in SEQ ID NO: 6, and
(ii) CDRL1, CDRL2 and CDRL3 comprised in a light chain variable region set forth in SEQ ID NO: 3, or
a light chain variable region set forth in SEQ ID NO: 3,
preferably, the CDRH1 is selected from SEQ ID NOs: 66, 72 and 77, the CDRH2 is selected from SEQ ID NOs: 67, 73 and 78, the CDRH3 is selected from SEQ ID NOs: 68 and 74, the CDRL1 is selected from SEQ ID NOs: 69 and 75, the CDRL2 is selected from SEQ ID NOs: 70 and 76, and the CDRL3 is set forth in SEQ ID NO: 71;
more preferably, the CDRH1 has a sequence set forth in SEQ ID NO: 66, the CDRH2 has a sequence set forth in SEQ ID NO: 67, the CDRH3 has a sequence set forth in SEQ ID NO: 68, the CDRL1 has a sequence set forth in SEQ ID NO: 69, the CDRL2 has a sequence set forth in SEQ ID NO: 70, and the CDRL3 has a sequence set forth in SEQ ID NO: 71;
more preferably, the CDRH1 has a sequence set forth in SEQ ID NO: 72, the CDRH2 has a sequence set forth in SEQ ID NO: 73, the CDRH3 has a sequence set forth in SEQ ID NO: 74, the CDRL1 has a sequence set forth in SEQ ID NO: 75, the CDRL2 has a sequence set forth in SEQ ID NO: 76, and the CDRL3 has a sequence set forth in SEQ ID NO: 71;
more preferably, the CDRH1 has a sequence set forth in SEQ ID NO: 77, the CDRH2 has a sequence set forth in SEQ ID NO: 78, the CDRH3 has a sequence set forth in SEQ ID NO: 74, the CDRL1 has a sequence set forth in SEQ ID NO: 75, the CDRL2 has a sequence set forth in SEQ ID NO: 76, and the CDRL3 has a sequence set forth in SEQ ID NO: 71; and wherein the antigen-binding domain specifically binding to CD3 is selected from the group consisting of:
1). an antigen-binding domain specifically binding to CD3 comprising the following CDRs (or variants thereof) or variable regions (or variants thereof):
(i) CDRH1, CDRH2 and CDRH3 comprised in a heavy chain variable region set forth in SEQ ID NO: 7, or
a heavy chain variable region set forth in SEQ ID NO: 7, and
(ii) CDRL1, CDRL2 and CDRL3 comprised in a light chain variable region set forth in SEQ ID NO: 9, or
a light chain variable region set forth in SEQ ID NO: 9,
preferably, the CDRH1 has a sequence set forth in SEQ ID NO: 79, the CDRH2 has a sequence set forth in SEQ ID NO: 80, the CDRH3 has a sequence set forth in SEQ ID NO: 81, the CDRL1 has a sequence set forth in SEQ ID NO: 82, the CDRL2 has a sequence set forth in SEQ ID NO: 83, and the CDRL3 has a sequence set forth in SEQ ID NO: 84;
2). an antigen-binding domain specifically binding to CD3 comprising the following CDRs (or variants thereof) or variable regions (or variants thereof):
(i) CDRH1, CDRH2 and CDRH3 comprised in a heavy chain variable region set forth in SEQ ID NO: 10, or
a heavy chain variable region set forth in SEQ ID NO: 10, and
(ii) CDRL1, CDRL2 and CDRL3 comprised in a light chain variable region set forth in SEQ ID NO: 11, or
a light chain variable region set forth in SEQ ID NO: 11,
preferably, the CDRH1 has a sequence set forth in SEQ ID NO: 79, the CDRH2 has a sequence set forth in SEQ ID NO: 80, the CDRH3 has a sequence set forth in SEQ ID NO: 85, the CDRL1 has a sequence set forth in SEQ ID NO: 82, the CDRL2 has a sequence set forth in SEQ ID NO: 83, and the CDRL3 has a sequence set forth in SEQ ID NO: 84;
3). an antigen-binding domain specifically binding to CD3 comprising the following CDRs (or variants thereof) or variable regions (or variants thereof):
(i) CDRH1, CDRH2 and CDRH3 comprised in a heavy chain variable region set forth in SEQ ID NO: 12, or
a heavy chain variable region set forth in SEQ ID NO: 12, and
(ii) CDRL1, CDRL2 and CDRL3 comprised in a light chain variable region set forth in SEQ ID NO: 13, or
a light chain variable region set forth in SEQ ID NO: 13,
preferably, the CDRH1 has a sequence set forth in SEQ ID NO: 79, the CDRH2 has a sequence set forth in SEQ ID NO: 80, the CDRH3 has a sequence set forth in SEQ ID NO: 85, the CDRL1 has a sequence set forth in SEQ ID NO: 82, the CDRL2 has a sequence set forth in SEQ ID NO: 83, and the CDRL3 has a sequence set forth in SEQ ID NO: 84;
4). an antigen-binding domain specifically binding to CD3 comprising the following CDRs (or variants thereof) or variable regions (or variants thereof):
(i) CDRH1, CDRH2 and CDRH3 comprised in a heavy chain variable region set forth in SEQ ID NO: 14, or
a heavy chain variable region set forth in SEQ ID NO: 14, and
(ii) CDRL1, CDRL2 and CDRL3 comprised in a light chain variable region set forth in SEQ ID NO: 15, or
a light chain variable region set forth in SEQ ID NO: 15,
preferably, the CDRH1 has a sequence set forth in SEQ ID NO: 86, the CDRH2 has a sequence set forth in SEQ ID NO: 87, the CDRH3 has a sequence set forth in SEQ ID NO: 88, the CDRL1 has a sequence set forth in SEQ ID NO: 89, the CDRL2 has a sequence set forth in SEQ ID NO: 90, and the CDRL3 has a sequence set forth in SEQ ID NO: 91; or
5). an antigen-binding domain specifically binding to CD3 comprising the following CDRs (or variants thereof) or variable regions (or variants thereof):
(i) CDRH1, CDRH2 and CDRH3 comprised in a heavy chain variable region set forth in SEQ ID NO: 16, or
a heavy chain variable region set forth in SEQ ID NO: 16, and
(ii) CDRL1, CDRL2 and CDRL3 comprised in a light chain variable region set forth in SEQ ID NO: 17, or
a light chain variable region set forth in SEQ ID NO: 17,
preferably, the CDRH1 has a sequence set forth in SEQ ID NO: 92, the CDRH2 has a sequence set forth in SEQ ID NO: 93, the CDRH3 has a sequence set forth in SEQ ID NO: 94, the CDRL1 has a sequence set forth in SEQ ID NO: 95, the CDRL2 has a sequence set forth in SEQ ID NO: 96, and the CDRL3 has a sequence set forth in SEQ ID NO: 97,
wherein the variants differ from the CDRs or the variable regions by 3, 2 or 1 amino acids or have at least 80%, 85%, 90%, 91%, 92%, 93%, 94%, 95%, 96%, 97%, 98% or 99% identity thereto.
5. The bispecific antibody according to item 1, comprising an antigen-binding domain specifically binding to CD3 and an antigen-binding domain specifically binding to CLDN18.2, wherein
(1) the antigen-binding domain specifically binding to CD3 comprises the following CDRs or variants thereof:
(i) CDRH1, CDRH2 and CDRH3 comprised in a heavy chain variable region set forth in SEQ ID NO: 7, and
(ii) CDRL1, CDRL2 and CDRL3 comprised in a light chain variable region set forth in SEQ ID NO: 9,
preferably, the CDRH1 has a sequence set forth in SEQ ID NO: 79, the CDRH2 has a sequence set forth in SEQ ID NO: 80, the CDRH3 has a sequence set forth in SEQ ID NO: 81, the CDRL1 has a sequence set forth in SEQ ID NO: 82, the CDRL2 has a sequence set forth in SEQ ID NO: 83, and the CDRL3 has a sequence set forth in SEQ ID NO: 84; and the antigen-binding domain specifically binding to CLDN18.2 comprises the following CDRs or variants thereof:
(i) CDRH1, CDRH2 and CDRH3 comprised in a heavy chain variable region set forth in SEQ ID NO: 4, and
(ii) CDRL1, CDRL2 and CDRL3 comprised in a light chain variable region set forth in SEQ ID NO: 1,
preferably, the CDRH1 has a sequence set forth in SEQ ID NO: 47, the CDRH2 has a sequence set forth in SEQ ID NO: 48, the CDRH3 has a sequence set forth in SEQ ID NO: 49, the CDRL1 has a sequence set forth in SEQ ID NO: 50, the CDRL2 has a sequence set forth in SEQ ID NO: 51, and the CDRL3 has a sequence set forth in SEQ ID NO: 52; or
(2) the antigen-binding domain specifically binding to CD3 comprises the following CDRs or variants thereof:
(i) CDRH1, CDRH2 and CDRH3 comprised in a heavy chain variable region set forth in SEQ ID NO: 10, and
(ii) CDRL1, CDRL2 and CDRL3 comprised in a light chain variable region set forth in SEQ ID NO: 11,
preferably, the CDRH1 has a sequence set forth in SEQ ID NO: 79, the CDRH2 has a sequence set forth in SEQ ID NO: 80, the CDRH3 has a sequence set forth in SEQ ID NO: 85, the CDRL1 has a sequence set forth in SEQ ID NO: 82, the CDRL2 has a sequence set forth in SEQ ID NO: 83, and the CDRL3 has a sequence set forth in SEQ ID NO: 84; and
the antigen-binding domain specifically binding to CLDN18.2 comprises the following CDRs or variants thereof:
(i) CDRH1, CDRH2 and CDRH3 comprised in a heavy chain variable region set forth in SEQ ID NO: 4, and
(ii) CDRL1, CDRL2 and CDRL3 comprised in a light chain variable region set forth in SEQ ID NO: 1,
preferably, the CDRH1 has a sequence set forth in SEQ ID NO: 47, the CDRH2 has a sequence set forth in SEQ ID NO: 48, the CDRH3 has a sequence set forth in SEQ ID NO: 49, the CDRL1 has a sequence set forth in SEQ ID NO: 50, the CDRL2 has a sequence set forth in SEQ ID NO: 51, and the CDRL3 has a sequence set forth in SEQ ID NO: 52;
or
(3) the antigen-binding domain specifically binding to CD3 comprises the following CDRs or variants thereof:
(i) CDRH1, CDRH2 and CDRH3 comprised in a heavy chain variable region set forth in SEQ ID NO: 12, and
(ii) CDRL1, CDRL2 and CDRL3 comprised in a light chain variable region set forth in SEQ ID NO: 13,
preferably, the CDRH1 has a sequence set forth in SEQ ID NO: 79, the CDRH2 has a sequence set forth in SEQ ID NO: 80, the CDRH3 has a sequence set forth in SEQ ID NO: 85, the CDRL1 has a sequence set forth in SEQ ID NO: 82, the CDRL2 has a sequence set forth in SEQ ID NO: 83, and the CDRL3 has a sequence set forth in SEQ ID NO: 84; and
the antigen-binding domain specifically binding to CLDN18.2 comprises the following CDRs or variants thereof:
(i) CDRH1, CDRH2 and CDRH3 comprised in a heavy chain variable region set forth in SEQ ID NO: 4, and
(ii) CDRL1, CDRL2 and CDRL3 comprised in a light chain variable region set forth in SEQ ID NO: 1,
preferably, the CDRH1 has a sequence set forth in SEQ ID NO: 47, the CDRH2 has a sequence set forth in SEQ ID NO: 48, the CDRH3 has a sequence set forth in SEQ ID NO: 49, the CDRL1 has a sequence set forth in SEQ ID NO: 50, the CDRL2 has a sequence set forth in SEQ ID NO: 51, and the CDRL3 has a sequence set forth in SEQ ID NO: 52;
or
(4) the antigen-binding domain specifically binding to CD3 comprises the following CDRs or variants thereof:
(i) CDRH1, CDRH2 and CDRH3 comprised in a heavy chain variable region set forth in SEQ ID NO: 14, and
(ii) CDRL1, CDRL2 and CDRL3 comprised in a light chain variable region set forth in SEQ ID NO: 15, preferably, the CDRH1 has a sequence set forth in SEQ ID NO: 86, the CDRH2 has a sequence set forth in SEQ ID NO: 87, the CDRH3 has a sequence set forth in SEQ ID NO: 88, the CDRL1 has a sequence set forth in SEQ ID NO: 89, the CDRL2 has a sequence set forth in SEQ ID NO: 90, and the CDRL3 has a sequence set forth in SEQ ID NO: 91; and
the antigen-binding domain specifically binding to CLDN18.2 comprises the following CDRs or variants thereof:
(i) CDRH1, CDRH2 and CDRH3 comprised in a heavy chain variable region set forth in SEQ ID NO: 4, and
(ii) CDRL1, CDRL2 and CDRL3 comprised in a light chain variable region set forth in SEQ ID NO: 1,
preferably, the CDRH1 has a sequence set forth in SEQ ID NO: 47, the CDRH2 has a sequence set forth in SEQ ID NO: 48, the CDRH3 has a sequence set forth in SEQ ID NO: 49, the CDRL1 has a sequence set forth in SEQ ID NO: 50, the CDRL2 has a sequence set forth in SEQ ID NO: 51, and the CDRL3 has a sequence set forth in SEQ ID NO: 52;
or
(5) the antigen-binding domain specifically binding to CD3 comprises the following CDRs or variants thereof:
(i) CDRH1, CDRH2 and CDRH3 comprised in a heavy chain variable region set forth in SEQ ID NO: 16, and
(ii) CDRL1, CDRL2 and CDRL3 comprised in a light chain variable region set forth in SEQ ID NO: 17,
preferably, the CDRH1 has a sequence set forth in SEQ ID NO: 92, the CDRH2 has a sequence set forth in SEQ ID NO: 93, the CDRH3 has a sequence set forth in SEQ ID NO: 94, the CDRL1 has a sequence set forth in SEQ ID NO: 95, the CDRL2 has a sequence set forth in SEQ ID NO: 96, and the CDRL3 has a sequence set forth in SEQ ID NO: 97; and
the antigen-binding domain specifically binding to CLDN18.2 comprises the following CDRs or variants thereof:
(i) CDRH1, CDRH2 and CDRH3 comprised in a heavy chain variable region set forth in SEQ ID NO: 4, and
(ii) CDRL1, CDRL2 and CDRL3 comprised in a light chain variable region set forth in SEQ ID NO: 1,
preferably, the CDRH1 has a sequence set forth in SEQ ID NO: 47, the CDRH2 has a sequence set forth in SEQ ID NO: 48, the CDRH3 has a sequence set forth in SEQ ID NO: 49, the CDRL1 has a sequence set forth in SEQ ID NO: 50, the CDRL2 has a sequence set forth in SEQ ID NO: 51, and the CDRL3 has a sequence set forth in SEQ ID NO: 52;
or
(6) wherein the antigen-binding domain specifically binding to CD3 comprises the following CDRs or variants thereof:
(i) CDRH1, CDRH2 and CDRH3 comprised in a heavy chain variable region set forth in SEQ ID NO: 7, and
(ii) CDRL1, CDRL2 and CDRL3 comprised in a light chain variable region set forth in SEQ ID NO: 9,
preferably, the CDRH1 has a sequence set forth in SEQ ID NO: 79, the CDRH2 has a sequence set forth in SEQ ID NO: 80, the CDRH3 has a sequence set forth in SEQ ID NO: 81, the CDRL1 has a sequence set forth in SEQ ID NO: 82, the CDRL2 has a sequence set forth in SEQ ID NO: 83, and the CDRL3 has a sequence set forth in SEQ ID NO: 84; and
the antigen-binding domain specifically binding to CLDN18.2 comprises the following CDRs or variants thereof:
(i) CDRH1, CDRH2 and CDRH3 comprised in a heavy chain variable region set forth in SEQ ID NO: 5, and
(ii) CDRL1, CDRL2 and CDRL3 comprised in a light chain variable region set forth in SEQ ID NO: 2,
preferably, the CDRH1 is selected from SEQ ID NOs: 53, 59 and 64, the CDRH2 is selected from SEQ ID NOs: 54, 60 and 65, the CDRH3 is selected from SEQ ID NOs: 55 and 61, the CDRL1 is selected from SEQ ID NOs: 56 and 62, the CDRL2 is selected from SEQ ID NOs: 57 and 63, and the CDRL3 is selected from SEQ ID NO: 58;
more preferably, the CDRH1 has a sequence set forth in SEQ ID NO: 53, the CDRH2 has a sequence set forth in SEQ ID NO: 54, the CDRH3 has a sequence set forth in SEQ ID NO: 55, the CDRL1 has a sequence set forth in SEQ ID NO: 56, the CDRL2 has a sequence set forth in SEQ ID NO: 57, and the CDRL3 has a sequence set forth in SEQ ID NO: 58;
more preferably, the CDRH1 has a sequence set forth in SEQ ID NO: 59, the CDRH2 has a sequence set forth in SEQ ID NO: 60, the CDRH3 has a sequence set forth in SEQ ID NO: 61, the CDRL1 has a sequence set forth in SEQ ID NO: 62, the CDRL2 has a sequence set forth in SEQ ID NO: 63, and the CDRL3 has a sequence set forth in SEQ ID NO: 58;
more preferably, the CDRH1 has a sequence set forth in SEQ ID NO: 64, the CDRH2 has a sequence set forth in SEQ ID NO: 65, the CDRH3 has a sequence set forth in SEQ ID NO: 61, the CDRL1 has a sequence set forth in SEQ ID NO: 62, the CDRL2 has a sequence set forth in SEQ ID NO: 63, and the CDRL3 has a sequence set forth in SEQ ID NO: 58;
or
(7) the antigen-binding domain specifically binding to CD3 comprises the following CDRs or variants thereof:
(i) CDRH1, CDRH2 and CDRH3 comprised in a heavy chain variable region set forth in SEQ ID NO: 10, and
(ii) CDRL1, CDRL2 and CDRL3 comprised in a light chain variable region set forth in SEQ ID NO: 11,
preferably, the CDRH1 has a sequence set forth in SEQ ID NO: 79, the CDRH2 has a sequence set forth in SEQ ID NO: 80, the CDRH3 has a sequence set forth in SEQ ID NO: 85, the CDRL1 has a sequence set forth in SEQ ID NO: 82, the CDRL2 has a sequence set forth in SEQ ID NO: 83, and the CDRL3 has a sequence set forth in SEQ ID NO: 84; and
the antigen-binding domain specifically binding to CLDN18.2 comprises the following CDRs or variants thereof:
(i) CDRH1, CDRH2 and CDRH3 comprised in a heavy chain variable region set forth in SEQ ID NO: 5, and
(ii) CDRL1, CDRL2 and CDRL3 comprised in a light chain variable region set forth in SEQ ID NO: 2,
preferably, the CDRH1 is selected from SEQ ID NOs: 53, 59 and 64, the CDRH2 is selected from SEQ ID NOs: 54, 60 and 65, the CDRH3 is selected from SEQ ID NOs: 55 and 61, the CDRL1 is selected from SEQ ID NOs: 56 and 62, the CDRL2 is selected from SEQ ID NOs: 57 and 63, and the CDRL3 is set forth in SEQ ID NO: 58;
more preferably, the CDRH1 has a sequence set forth in SEQ ID NO: 53, the CDRH2 has a sequence set forth in SEQ ID NO: 54, the CDRH3 has a sequence set forth in SEQ ID NO: 55, the CDRL1 has a sequence set forth in SEQ ID NO: 56, the CDRL2 has a sequence set forth in SEQ ID NO: 57, and the CDRL3 has a sequence set forth in SEQ ID NO: 58;
more preferably, the CDRH1 has a sequence set forth in SEQ ID NO: 59, the CDRH2 has a sequence set forth in SEQ ID NO: 60, the CDRH3 has a sequence set forth in SEQ ID NO: 61, the CDRL1 has a sequence set forth in SEQ ID NO: 62, the CDRL2 has a sequence set forth in SEQ ID NO: 63, and the CDRL3 has a sequence set forth in SEQ ID NO: 58;
more preferably, the CDRH1 has a sequence set forth in SEQ ID NO: 64, the CDRH2 has a sequence set forth in SEQ ID NO: 65, the CDRH3 has a sequence set forth in SEQ ID NO: 61, the CDRL1 has a sequence set forth in SEQ ID NO: 62, the CDRL2 has a sequence set forth in SEQ ID NO: 63, and the CDRL3 has a sequence set forth in SEQ ID NO: 58;
or
(8) the antigen-binding domain specifically binding to CD3 comprises the following CDRs or variants thereof:
(i) CDRH1, CDRH2 and CDRH3 comprised in a heavy chain variable region set forth in SEQ ID NO: 12, and
(ii) CDRL1, CDRL2 and CDRL3 comprised in a light chain variable region set forth in SEQ ID NO: 13,
preferably, the CDRH1 has a sequence set forth in SEQ ID NO: 79, the CDRH2 has a sequence set forth in SEQ ID NO: 80, the CDRH3 has a sequence set forth in SEQ ID NO: 85, the CDRL1 has a sequence set forth in SEQ ID NO: 82, the CDRL2 has a sequence set forth in SEQ ID NO: 83, and the CDRL3 has a sequence set forth in SEQ ID NO: 84; and
the antigen-binding domain specifically binding to CLDN18.2 comprises the following CDRs or variants thereof:
(i) CDRH1, CDRH2 and CDRH3 comprised in a heavy chain variable region set forth in SEQ ID NO: 5, and
(ii) CDRL1, CDRL2 and CDRL3 comprised in a light chain variable region set forth in SEQ ID NO: 2,
preferably, the CDRH1 is selected from SEQ ID NOs: 53, 59 and 64, the CDRH2 is selected from SEQ ID NOs: 54, 60 and 65, the CDRH3 is selected from SEQ ID NOs: 55 and 61, the CDRL1 is selected from SEQ ID NOs: 56 and 62, the CDRL2 is selected from SEQ ID NOs: 57 and 63, and the CDRL3 is set forth in SEQ ID NO: 58;
more preferably, the CDRH1 has a sequence set forth in SEQ ID NO: 53, the CDRH2 has a sequence set forth in SEQ ID NO: 54, the CDRH3 has a sequence set forth in SEQ ID NO: 55, the CDRL1 has a sequence set forth in SEQ ID NO: 56, the CDRL2 has a sequence set forth in SEQ ID NO: 57, and the CDRL3 has a sequence set forth in SEQ ID NO: 58;
more preferably, the CDRH1 has a sequence set forth in SEQ ID NO: 59, the CDRH2 has a sequence set forth in SEQ ID NO: 60, the CDRH3 has a sequence set forth in SEQ ID NO: 61, the CDRL1 has a sequence set forth in SEQ ID NO: 62, the CDRL2 has a sequence set forth in SEQ ID NO: 63, and the CDRL3 has a sequence set forth in SEQ ID NO: 58;
more preferably, the CDRH1 has a sequence set forth in SEQ ID NO: 64, the CDRH2 has a sequence set forth in SEQ ID NO: 65, the CDRH3 has a sequence set forth in SEQ ID NO: 61, the CDRL1 has a sequence set forth in SEQ ID NO: 62, the CDRL2 has a sequence set forth in SEQ ID NO: 63, and the CDRL3 has a sequence set forth in SEQ ID NO: 58;
or
(9) the antigen-binding domain specifically binding to CD3 comprises the following CDRs or variants thereof:
(i) CDRH1, CDRH2 and CDRH3 comprised in a heavy chain variable region set forth in SEQ ID NO: 14, and
(ii) CDRL1, CDRL2 and CDRL3 comprised in a light chain variable region set forth in SEQ ID NO: 15,
preferably, the CDRH1 has a sequence set forth in SEQ ID NO: 86, the CDRH2 has a sequence set forth in SEQ ID NO: 87, the CDRH3 has a sequence set forth in SEQ ID NO: 88, the CDRL1 has a sequence set forth in SEQ ID NO: 89, the CDRL2 has a sequence set forth in SEQ ID NO: 90, and the CDRL3 has a sequence set forth in SEQ ID NO: 91; and
the antigen-binding domain specifically binding to CLDN18.2 comprises the following CDRs or variants thereof:
(i) CDRH1, CDRH2 and CDRH3 comprised in a heavy chain variable region set forth in SEQ ID NO: 5, and
(ii) CDRL1, CDRL2 and CDRL3 comprised in a light chain variable region set forth in SEQ ID NO: 2,
preferably, the CDRH1 is selected from SEQ ID NOs: 53, 59 and 64, the CDRH2 is selected from SEQ ID NOs: 54, 60 and 65, the CDRH3 is selected from SEQ ID NOs: 55 and 61, the CDRL1 is selected from SEQ ID NOs: 56 and 62, the CDRL2 is selected from SEQ ID NOs: 57 and 63, and the CDRL3 is set forth in SEQ ID NO: 58;
more preferably, the CDRH1 has a sequence set forth in SEQ ID NO: 53, the CDRH2 has a sequence set forth in SEQ ID NO: 54, the CDRH3 has a sequence set forth in SEQ ID NO: 55, the CDRL1 has a sequence set forth in SEQ ID NO: 56, the CDRL2 has a sequence set forth in SEQ ID NO: 57, and the CDRL3 has a sequence set forth in SEQ ID NO: 58;
more preferably, the CDRH1 has a sequence set forth in SEQ ID NO: 59, the CDRH2 has a sequence set forth in SEQ ID NO: 60, the CDRH3 has a sequence set forth in SEQ ID NO: 61, the CDRL1 has a sequence set forth in SEQ ID NO: 62, the CDRL2 has a sequence set forth in SEQ ID NO: 63, and the CDRL3 has a sequence set forth in SEQ ID NO: 58;
more preferably, the CDRH1 has a sequence set forth in SEQ ID NO: 64, the CDRH2 has a sequence set forth in SEQ ID NO: 65, the CDRH3 has a sequence set forth in SEQ ID NO: 61, the CDRL1 has a sequence set forth in SEQ ID NO: 62, the CDRL2 has a sequence set forth in SEQ ID NO: 63, and the CDRL3 has a sequence set forth in SEQ ID NO: 58; or
(10) the antigen-binding domain specifically binding to CD3 comprises the following CDRs or variants thereof:
(i) CDRH1, CDRH2 and CDRH3 comprised in a heavy chain variable region set forth in SEQ ID NO: 16, and
(ii) CDRL1, CDRL2 and CDRL3 comprised in a light chain variable region set forth in SEQ ID NO: 17,
preferably, the CDRH1 has a sequence set forth in SEQ ID NO: 92, the CDRH2 has a sequence set forth in SEQ ID NO: 93, the CDRH3 has a sequence set forth in SEQ ID NO: 94, the CDRL1 has a sequence set forth in SEQ ID NO: 95, the CDRL2 has a sequence set forth in SEQ ID NO: 96, and the CDRL3 has a sequence set forth in SEQ ID NO: 97; and
the antigen-binding domain specifically binding to CLDN18.2 comprises the following CDRs or variants thereof:
(i) CDRH1, CDRH2 and CDRH3 comprised in a heavy chain variable region set forth in SEQ ID NO: 5, and
(ii) CDRL1, CDRL2 and CDRL3 comprised in a light chain variable region set forth in SEQ ID NO: 2,
preferably, the CDRH1 is selected from SEQ ID NOs: 53, 59 and 64, the CDRH2 is selected from SEQ ID NOs: 54, 60 and 65, the CDRH3 is selected from SEQ ID NOs: 55 and 61, the CDRL1 is selected from SEQ ID NOs: 56 and 62, the CDRL2 is selected from SEQ ID NOs: 57 and 63, and the CDRL3 is set forth in SEQ ID NO: 58;
more preferably, the CDRH1 has a sequence set forth in SEQ ID NO: 53, the CDRH2 has a sequence set forth in SEQ ID NO: 54, the CDRH3 has a sequence set forth in SEQ ID NO: 55, the CDRL1 has a sequence set forth in SEQ ID NO: 56, the CDRL2 has a sequence set forth in SEQ ID NO: 57, and the CDRL3 has a sequence set forth in SEQ ID NO: 58;
more preferably, the CDRH1 has a sequence set forth in SEQ ID NO: 59, the CDRH2 has a sequence set forth in SEQ ID NO: 60, the CDRH3 has a sequence set forth in SEQ ID NO: 61, the CDRL1 has a sequence set forth in SEQ ID NO: 62, the CDRL2 has a sequence set forth in SEQ ID NO: 63, and the CDRL3 has a sequence set forth in SEQ ID NO: 58;
more preferably, the CDRH1 has a sequence set forth in SEQ ID NO: 64, the CDRH2 has a sequence set forth in SEQ ID NO: 65, the CDRH3 has a sequence set forth in SEQ ID NO: 61, the CDRL1 has a sequence set forth in SEQ ID NO: 62, the CDRL2 has a sequence set forth in SEQ ID NO: 63, and the CDRL3 has a sequence set forth in SEQ ID NO: 58; or
(11) wherein the antigen-binding domain specifically binding to CD3 comprises the following CDRs or variants thereof:
(i) CDRH1, CDRH2 and CDRH3 comprised in a heavy chain variable region set forth in SEQ ID NO: 7, and
(ii) CDRL1, CDRL2 and CDRL3 comprised in a light chain variable region set forth in SEQ ID NO: 9,
preferably, the CDRH1 has a sequence set forth in SEQ ID NO: 79, the CDRH2 has a sequence set forth in SEQ ID NO: 80, the CDRH3 has a sequence set forth in SEQ ID NO: 81, the CDRL1 has a sequence set forth in SEQ ID NO: 82, the CDRL2 has a sequence set forth in SEQ ID NO: 83, and the CDRL3 has a sequence set forth in SEQ ID NO: 84; and
the antigen-binding domain specifically binding to CLDN18.2 comprises the following CDRs or variants thereof:
(i) CDRH1, CDRH2 and CDRH3 comprised in a heavy chain variable region set forth in SEQ ID NO: 6, and
(ii) CDRL1, CDRL2 and CDRL3 comprised in a light chain variable region set forth in SEQ ID NO: 3,
preferably, the CDRH1 is selected from SEQ ID NOs: 66, 72 and 77, the CDRH2 is selected from SEQ ID NOs: 67, 73 and 78, the CDRH3 is selected from SEQ ID NOs: 68 and 74, the CDRL1 is selected from SEQ ID NOs: 69 and 75, the CDRL2 is selected from SEQ ID NOs: 70 and 76, and the CDRL3 is set forth in SEQ ID NO: 71;
more preferably, the CDRH1 has a sequence set forth in SEQ ID NO: 66, the CDRH2 has a sequence set forth in SEQ ID NO: 67, the CDRH3 has a sequence set forth in SEQ ID NO: 68, the CDRL1 has a sequence set forth in SEQ ID NO: 69, the CDRL2 has a sequence set forth in SEQ ID NO: 70, and the CDRL3 has a sequence set forth in SEQ ID NO: 71;
more preferably, the CDRH1 has a sequence set forth in SEQ ID NO: 72, the CDRH2 has a sequence set forth in SEQ ID NO: 73, the CDRH3 has a sequence set forth in SEQ ID NO: 74, the CDRL1 has a sequence set forth in SEQ ID NO: 75, the CDRL2 has a sequence set forth in SEQ ID NO: 76, and the CDRL3 has a sequence set forth in SEQ ID NO: 71;
more preferably, the CDRH1 has a sequence set forth in SEQ ID NO: 77, the CDRH2 has a sequence set forth in SEQ ID NO: 78, the CDRH3 has a sequence set forth in SEQ ID NO: 74, the CDRL1 has a sequence set forth in SEQ ID NO: 75, the CDRL2 has a sequence set forth in SEQ ID NO: 76, and the CDRL3 has a sequence set forth in SEQ ID NO: 71;
or
(12) the antigen-binding domain specifically binding to CD3 comprises the following CDRs or variants thereof:
(i) CDRH1, CDRH2 and CDRH3 comprised in a heavy chain variable region set forth in SEQ ID NO: 10, and
(ii) CDRL1, CDRL2 and CDRL3 comprised in a light chain variable region set forth in SEQ ID NO: 11,
preferably, the CDRH1 has a sequence set forth in SEQ ID NO: 79, the CDRH2 has a sequence set forth in SEQ ID NO: 80, the CDRH3 has a sequence set forth in SEQ ID NO: 85, the CDRL1 has a sequence set forth in SEQ ID NO: 82, the CDRL2 has a sequence set forth in SEQ ID NO: 83, and the CDRL3 has a sequence set forth in SEQ ID NO: 84; and
the antigen-binding domain specifically binding to CLDN18.2 comprises the following CDRs or variants thereof:
(i) CDRH1, CDRH2 and CDRH3 comprised in a heavy chain variable region set forth in SEQ ID NO: 6, and
(ii) CDRL1, CDRL2 and CDRL3 comprised in a light chain variable region set forth in SEQ ID NO: 3,
preferably, the CDRH1 is selected from SEQ ID NOs: 66, 72 and 77, the CDRH2 is selected from SEQ ID NOs: 67, 73 and 78, the CDRH3 is selected from SEQ ID NOs: 68 and 74, the CDRL1 is selected from SEQ ID NOs: 69 and 75, the CDRL2 is selected from SEQ ID NOs: 70 and 76, and the CDRL3 is set forth in SEQ ID NO: 71;
more preferably, the CDRH1 has a sequence set forth in SEQ ID NO: 66, the CDRH2 has a sequence set forth in SEQ ID NO: 67, the CDRH3 has a sequence set forth in SEQ ID NO: 68, the CDRL1 has a sequence set forth in SEQ ID NO: 69, the CDRL2 has a sequence set forth in SEQ ID NO: 70, and the CDRL3 has a sequence set forth in SEQ ID NO: 71;
more preferably, the CDRH1 has a sequence set forth in SEQ ID NO: 72, the CDRH2 has a sequence set forth in SEQ ID NO: 73, the CDRH3 has a sequence set forth in SEQ ID NO: 74, the CDRL1 has a sequence set forth in SEQ ID NO: 75, the CDRL2 has a sequence set forth in SEQ ID NO: 76, and the CDRL3 has a sequence set forth in SEQ ID NO: 71;
more preferably, the CDRH1 has a sequence set forth in SEQ ID NO: 77, the CDRH2 has a sequence set forth in SEQ ID NO: 78, the CDRH3 has a sequence set forth in SEQ ID NO: 74, the CDRL1 has a sequence set forth in SEQ ID NO: 75, the CDRL2 has a sequence set forth in SEQ ID NO: 76, and the CDRL3 has a sequence set forth in SEQ ID NO: 71;
or
(13) the antigen-binding domain specifically binding to CD3 comprises the following CDRs or variants thereof:
(i) CDRH1, CDRH2 and CDRH3 comprised in a heavy chain variable region set forth in SEQ ID NO: 12, and
(ii) CDRL1, CDRL2 and CDRL3 comprised in a light chain variable region set forth in SEQ ID NO: 13,
preferably, the CDRH1 has a sequence set forth in SEQ ID NO: 79, the CDRH2 has a sequence set forth in SEQ ID NO: 80, the CDRH3 has a sequence set forth in SEQ ID NO: 85, the CDRL1 has a sequence set forth in SEQ ID NO: 82, the CDRL2 has a sequence set forth in SEQ ID NO: 83, and the CDRL3 has a sequence set forth in SEQ ID NO: 84; and
the antigen-binding domain specifically binding to CLDN18.2 comprises the following CDRs or variants thereof:
(i) CDRH1, CDRH2 and CDRH3 comprised in a heavy chain variable region set forth in SEQ ID NO: 6, and
(ii) CDRL1, CDRL2 and CDRL3 comprised in a light chain variable region set forth in SEQ ID NO: 3,
preferably, the CDRH1 is selected from SEQ ID NOs: 66, 72 and 77, the CDRH2 is selected from SEQ ID NOs: 67, 73 and 78, the CDRH3 is selected from SEQ ID NOs: 68 and 74, the CDRL1 is selected from SEQ ID NOs: 69 and 75, the CDRL2 is selected from SEQ ID NOs: 70 and 76, and the CDRL3 is set forth in SEQ ID NO: 71;
more preferably, the CDRH1 has a sequence set forth in SEQ ID NO: 66, the CDRH2 has a sequence set forth in SEQ ID NO: 67, the CDRH3 has a sequence set forth in SEQ ID NO: 68, the CDRL1 has a sequence set forth in SEQ ID NO: 69, the CDRL2 has a sequence set forth in SEQ ID NO: 70, and the CDRL3 has a sequence set forth in SEQ ID NO: 71;
more preferably, the CDRH1 has a sequence set forth in SEQ ID NO: 72, the CDRH2 has a sequence set forth in SEQ ID NO: 73, the CDRH3 has a sequence set forth in SEQ ID NO: 74, the CDRL1 has a sequence set forth in SEQ ID NO: 75, the CDRL2 has a sequence set forth in SEQ ID NO: 76, and the CDRL3 has a sequence set forth in SEQ ID NO: 71;
more preferably, the CDRH1 has a sequence set forth in SEQ ID NO: 77, the CDRH2 has a sequence set forth in SEQ ID NO: 78, the CDRH3 has a sequence set forth in SEQ ID NO: 74, the CDRL1 has a sequence set forth in SEQ ID NO: 75, the CDRL2 has a sequence set forth in SEQ ID NO: 76, and the CDRL3 has a sequence set forth in SEQ ID NO: 71;
or
(14) the antigen-binding domain specifically binding to CD3 comprises the following CDRs or variants thereof:
(i) CDRH1, CDRH2 and CDRH3 comprised in a heavy chain variable region set forth in SEQ ID NO: 14, and
(ii) CDRL1, CDRL2 and CDRL3 comprised in a light chain variable region set forth in SEQ ID NO: 15,
preferably, the CDRH1 has a sequence set forth in SEQ ID NO: 86, the CDRH2 has a sequence set forth in SEQ ID NO: 87, the CDRH3 has a sequence set forth in SEQ ID NO: 88, the CDRL1 has a sequence set forth in SEQ ID NO: 89, the CDRL2 has a sequence set forth in SEQ ID NO: 90, and the CDRL3 has a sequence set forth in SEQ ID NO: 91; and
the antigen-binding domain specifically binding to CLDN18.2 comprises the following CDRs or variants thereof:
(i) CDRH1, CDRH2 and CDRH3 comprised in a heavy chain variable region set forth in SEQ ID NO: 6, and
(ii) CDRL1, CDRL2 and CDRL3 comprised in a light chain variable region set forth in SEQ ID NO: 3,
preferably, the CDRH1 is selected from SEQ ID NOs: 66, 72 and 77, the CDRH2 is selected from SEQ ID NOs: 67, 73 and 78, the CDRH3 is selected from SEQ ID NOs: 68 and 74, the CDRL1 is selected from SEQ ID NOs: 69 and 75, the CDRL2 is selected from SEQ ID NOs: 70 and 76, and the CDRL3 is set forth in SEQ ID NO: 71;
more preferably, the CDRH1 has a sequence set forth in SEQ ID NO: 66, the CDRH2 has a sequence set forth in SEQ ID NO: 67, the CDRH3 has a sequence set forth in SEQ ID NO: 68, the CDRL1 has a sequence set forth in SEQ ID NO: 69, the CDRL2 has a sequence set forth in SEQ ID NO: 70, and the CDRL3 has a sequence set forth in SEQ ID NO: 71;
more preferably, the CDRH1 has a sequence set forth in SEQ ID NO: 72, the CDRH2 has a sequence set forth in SEQ ID NO: 73, the CDRH3 has a sequence set forth in SEQ ID NO: 74, the CDRL1 has a sequence set forth in SEQ ID NO: 75, the CDRL2 has a sequence set forth in SEQ ID NO: 76, and the CDRL3 has a sequence set forth in SEQ ID NO: 71;
more preferably, the CDRH1 has a sequence set forth in SEQ ID NO: 77, the CDRH2 has a sequence set forth in SEQ ID NO: 78, the CDRH3 has a sequence set forth in SEQ ID NO: 74, the CDRL1 has a sequence set forth in SEQ ID NO: 75, the CDRL2 has a sequence set forth in SEQ ID NO: 76, and the CDRL3 has a sequence set forth in SEQ ID NO: 71; or
(15) the antigen-binding domain specifically binding to CD3 comprises the following CDRs or variants thereof:
(i) CDRH1, CDRH2 and CDRH3 comprised in a heavy chain variable region set forth in SEQ ID NO: 16, and
(ii) CDRL1, CDRL2 and CDRL3 comprised in a light chain variable region set forth in SEQ ID NO: 17,
preferably, the CDRH1 has a sequence set forth in SEQ ID NO: 92, the CDRH2 has a sequence set forth in SEQ ID NO: 93, the CDRH3 has a sequence set forth in SEQ ID NO: 94, the CDRL1 has a sequence set forth in SEQ ID NO: 95, the CDRL2 has a sequence set forth in SEQ ID NO: 96, and the CDRL3 has a sequence set forth in SEQ ID NO: 97; and
the antigen-binding domain specifically binding to CLDN18.2 comprises the following CDRs or variants thereof:
(i) CDRH1, CDRH2 and CDRH3 comprised in a heavy chain variable region set forth in SEQ ID NO: 6, and
(ii) CDRL1, CDRL2 and CDRL3 comprised in a light chain variable region set forth in SEQ ID NO: 3,
preferably, the CDRH1 is selected from SEQ ID NOs: 66, 72 and 77, the CDRH2 is selected from SEQ ID NOs: 67, 73 and 78, the CDRH3 is selected from SEQ ID NOs: 68 and 74, the CDRL1 is selected from SEQ ID NOs: 69 and 75, the CDRL2 is selected from SEQ ID NOs: 70 and 76, and the CDRL3 is set forth in SEQ ID NO: 71;
more preferably, the CDRH1 has a sequence set forth in SEQ ID NO: 66, the CDRH2 has a sequence set forth in SEQ ID NO: 67, the CDRH3 has a sequence set forth in SEQ ID NO: 68, the CDRL1 has a sequence set forth in SEQ ID NO: 69, the CDRL2 has a sequence set forth in SEQ ID NO: 70, and the CDRL3 has a sequence set forth in SEQ ID NO: 71;
more preferably, the CDRH1 has a sequence set forth in SEQ ID NO: 72, the CDRH2 has a sequence set forth in SEQ ID NO: 73, the CDRH3 has a sequence set forth in SEQ ID NO: 74, the CDRL1 has a sequence set forth in SEQ ID NO: 75, the CDRL2 has a sequence set forth in SEQ ID NO: 76, and the CDRL3 has a sequence set forth in SEQ ID NO: 71;
more preferably, the CDRH1 has a sequence set forth in SEQ ID NO: 77, the CDRH2 has a sequence set forth in SEQ ID NO: 78, the CDRH3 has a sequence set forth in SEQ ID NO: 74, the CDRL1 has a sequence set forth in SEQ ID NO: 75, the CDRL2 has a sequence set forth in SEQ ID NO: 76, and the CDRL3 has a sequence set forth in SEQ ID NO: 71;
wherein the variants differ from the CDRs by 3, 2 or 1 amino acids or have at least 80%, 85%, 90%, 91%, 92%, 93%, 94%, 95%, 96%, 97%, 98% or 99% identity thereto.
6. The bispecific antibody according to item 1, wherein the antigen-binding domain specifically binding to CD3 is selected from the group consisting of:
1). an antigen-binding domain specifically binding to CD3 comprising the following variable region amino acid sequences or variants thereof:
(i) a heavy chain variable region amino acid sequence set forth in SEQ ID NO: 7, and
(ii) a light chain variable region amino acid sequence set forth in SEQ ID NO: 9;
2). an antigen-binding domain specifically binding to CD3 comprising the following variable region amino acid sequences or variants thereof:
(i) a heavy chain variable region amino acid sequence set forth in SEQ ID NO: 10, and
(ii) a light chain variable region amino acid sequence set forth in SEQ ID NO: 11;
3). an antigen-binding domain specifically binding to CD3 comprising the following variable region amino acid sequences or variants thereof:
(i) a heavy chain variable region amino acid sequence set forth in SEQ ID NO: 12, and
(ii) a light chain variable region amino acid sequence set forth in SEQ ID NO: 13;
4). an antigen-binding domain specifically binding to CD3 comprising the following variable region amino acid sequences or variants thereof:
(i) a heavy chain variable region amino acid sequence set forth in SEQ ID NO: 14, and
(ii) a light chain variable region amino acid sequence set forth in SEQ ID NO: 15; or
5). an antigen-binding domain specifically binding to CD3 comprising the following variable region amino acid sequences or variants thereof:
(i) a heavy chain variable region amino acid sequence set forth in SEQ ID NO: 16, and
(ii) a light chain variable region amino acid sequence set forth in SEQ ID NO: 17;
wherein the antigen-binding domain specifically binding to CLDN18.2 is selected from the group consisting of:
1) an antigen-binding domain specifically binding to CLDN18.2 comprising the following variable region amino acid sequences or variants thereof:
(i) a heavy chain variable region amino acid sequence set forth in SEQ ID NO: 4, and
(ii) a light chain variable region amino acid sequence set forth in SEQ ID NO: 1;
2) an antigen-binding domain specifically binding to CLDN18.2 comprising the following variable region amino acid sequences or variants thereof:
(i) a heavy chain variable region amino acid sequence set forth in SEQ ID NO: 5, and
(ii) a light chain variable region amino acid sequence set forth in SEQ ID NO: 2; or
3) an antigen-binding domain specifically binding to CLDN18.2 comprising the following variable region amino acid sequences or variants thereof:
(i) a heavy chain variable region amino acid sequence set forth in SEQ ID NO: 6, and
(ii) a light chain variable region amino acid sequence set forth in SEQ ID NO: 3,
wherein the variants differ from the variable region amino acid sequences by 3, 2 or 1 amino acids or have at least 80%, 85%, 90%, 91%, 92%, 93%, 94%, 95%, 96%, 97%, 98% or 99% identity thereto.
7. The bispecific antibody according to item 1, comprising an antigen-binding domain specifically binding to CD3 and an antigen-binding domain specifically binding to CLDN18.2, wherein
(1) the antigen-binding domain specifically binding to CD3 comprises the following variable region amino acid sequences or variants thereof:
(i) a heavy chain variable region amino acid sequence set forth in SEQ ID NO: 7, and
(ii) a light chain variable region amino acid sequence set forth in SEQ ID NO: 9; and
the antigen-binding domain specifically binding to CLDN18.2 comprises the following variable region amino acid sequences or variants thereof:
(i) a heavy chain variable region amino acid sequence set forth in SEQ ID NO: 4, and
(ii) a light chain variable region amino acid sequence set forth in SEQ ID NO: 1; or
(2) the antigen-binding domain specifically binding to CD3 comprises the following variable region amino acid sequences or variants thereof:
(i) a heavy chain variable region amino acid sequence set forth in SEQ ID NO: 10, and
(ii) a light chain variable region amino acid sequence set forth in SEQ ID NO: 11; and
the antigen-binding domain specifically binding to CLDN18.2 comprises the following variable region amino acid sequences or variants thereof:
(i) a heavy chain variable region amino acid sequence set forth in SEQ ID NO: 4, and
(ii) a light chain variable region amino acid sequence set forth in SEQ ID NO: 1; or
(3) the antigen-binding domain specifically binding to CD3 comprises the following variable region amino acid sequences or variants thereof:
(i) a heavy chain variable region amino acid sequence set forth in SEQ ID NO: 12, and
(ii) a light chain variable region amino acid sequence set forth in SEQ ID NO: 13; and
the antigen-binding domain specifically binding to CLDN18.2 comprises the following variable region amino acid sequences or variants thereof:
(i) a heavy chain variable region amino acid sequence set forth in SEQ ID NO: 4, and
(ii) a light chain variable region amino acid sequence set forth in SEQ ID NO: 1; or
(4) the antigen-binding domain specifically binding to CD3 comprises the following variable region amino acid sequences or variants thereof:
(i) a heavy chain variable region amino acid sequence set forth in SEQ ID NO: 14, and
(ii) a light chain variable region amino acid sequence set forth in SEQ ID NO: 15; and
the antigen-binding domain specifically binding to CLDN18.2 comprises the following variable region amino acid sequences or variants thereof:
(i) a heavy chain variable region amino acid sequence set forth in SEQ ID NO: 4, and
(ii) a light chain variable region amino acid sequence set forth in SEQ ID NO: 1; or
(5) the antigen-binding domain specifically binding to CD3 comprises the following variable region amino acid sequences or variants thereof:
(i) a heavy chain variable region amino acid sequence set forth in SEQ ID NO: 16, and
(ii) a light chain variable region amino acid sequence set forth in SEQ ID NO: 17; and
the antigen-binding domain specifically binding to CLDN18.2 comprises the following variable region amino acid sequences or variants thereof:
(i) a heavy chain variable region amino acid sequence set forth in SEQ ID NO: 4, and
(ii) a light chain variable region amino acid sequence set forth in SEQ ID NO: 1; or
(6) the antigen-binding domain specifically binding to CD3 comprises the following variable region amino acid sequences or variants thereof:
(i) a heavy chain variable region amino acid sequence set forth in SEQ ID NO: 7, and
(ii) a light chain variable region amino acid sequence set forth in SEQ ID NO: 9; and
the antigen-binding domain specifically binding to CLDN18.2 comprises the following variable region amino acid sequences or variants thereof:
(i) a heavy chain variable region amino acid sequence set forth in SEQ ID NO: 5, and
(ii) a light chain variable region amino acid sequence set forth in SEQ ID NO: 2; or
(7) the antigen-binding domain specifically binding to CD3 comprises the following variable region amino acid sequences or variants thereof:
(i) a heavy chain variable region amino acid sequence set forth in SEQ ID NO: 10, and
(ii) a light chain variable region amino acid sequence set forth in SEQ ID NO: 11; and
the antigen-binding domain specifically binding to CLDN18.2 comprises the following variable region amino acid sequences or variants thereof:
(i) a heavy chain variable region amino acid sequence set forth in SEQ ID NO: 5, and
(ii) a light chain variable region amino acid sequence set forth in SEQ ID NO: 2; or
(8) the antigen-binding domain specifically binding to CD3 comprises the following variable region amino acid sequences or variants thereof:
(i) a heavy chain variable region amino acid sequence set forth in SEQ ID NO: 12, and
(ii) a light chain variable region amino acid sequence set forth in SEQ ID NO: 13; and
the antigen-binding domain specifically binding to CLDN18.2 comprises the following variable region amino acid sequences or variants thereof:
(i) a heavy chain variable region amino acid sequence set forth in SEQ ID NO: 5, and
(ii) a light chain variable region amino acid sequence set forth in SEQ ID NO: 2; or
(9) the antigen-binding domain specifically binding to CD3 comprises the following variable region amino acid sequences or variants thereof:
(i) a heavy chain variable region amino acid sequence set forth in SEQ ID NO: 14, and
(ii) a light chain variable region amino acid sequence set forth in SEQ ID NO: 15; and
the antigen-binding domain specifically binding to CLDN18.2 comprises the following variable region amino acid sequences or variants thereof:
(i) a heavy chain variable region amino acid sequence set forth in SEQ ID NO: 5, and
(ii) a light chain variable region amino acid sequence set forth in SEQ ID NO: 2; or
(10) the antigen-binding domain specifically binding to CD3 comprises the following variable region amino acid sequences or variants thereof:
(i) a heavy chain variable region amino acid sequence set forth in SEQ ID NO: 16, and
(ii) a light chain variable region amino acid sequence set forth in SEQ ID NO: 17; and
the antigen-binding domain specifically binding to CLDN18.2 comprises the following variable region amino acid sequences or variants thereof:
(i) a heavy chain variable region amino acid sequence set forth in SEQ ID NO: 5, and
(ii) a light chain variable region amino acid sequence set forth in SEQ ID NO: 2; or
(11) the antigen-binding domain specifically binding to CD3 comprises the following variable region amino acid sequences or variants thereof:
(i) a heavy chain variable region amino acid sequence set forth in SEQ ID NO: 7, and
(ii) a light chain variable region amino acid sequence set forth in SEQ ID NO: 9; and
the antigen-binding domain specifically binding to CLDN18.2 comprises the following variable region amino acid sequences or variants thereof:
(i) a heavy chain variable region amino acid sequence set forth in SEQ ID NO: 6, and
(ii) a light chain variable region amino acid sequence set forth in SEQ ID NO: 3; or
(12) the antigen-binding domain specifically binding to CD3 comprises the following variable region amino acid sequences or variants thereof:
(i) a heavy chain variable region amino acid sequence set forth in SEQ ID NO: 10, and
(ii) a light chain variable region amino acid sequence set forth in SEQ ID NO: 11; and
the antigen-binding domain specifically binding to CLDN18.2 comprises the following variable region amino acid sequences or variants thereof:
(i) a heavy chain variable region amino acid sequence set forth in SEQ ID NO: 6, and
(ii) a light chain variable region amino acid sequence set forth in SEQ ID NO: 3; or
(13) the antigen-binding domain specifically binding to CD3 comprises the following variable region amino acid sequences or variants thereof:
(i) a heavy chain variable region amino acid sequence set forth in SEQ ID NO: 12, and
(ii) a light chain variable region amino acid sequence set forth in SEQ ID NO: 13; and
the antigen-binding domain specifically binding to CLDN18.2 comprises the following variable region amino acid sequences or variants thereof:
(i) a heavy chain variable region amino acid sequence set forth in SEQ ID NO: 6, and
(ii) a light chain variable region amino acid sequence set forth in SEQ ID NO: 3; or
(14) the antigen-binding domain specifically binding to CD3 comprises the following variable region amino acid sequences or variants thereof:
(i) a heavy chain variable region amino acid sequence set forth in SEQ ID NO: 14, and
(ii) a light chain variable region amino acid sequence set forth in SEQ ID NO: 15; and
the antigen-binding domain specifically binding to CLDN18.2 comprises the following variable region amino acid sequences or variants thereof:
(i) a heavy chain variable region amino acid sequence set forth in SEQ ID NO: 6, and
(ii) a light chain variable region amino acid sequence set forth in SEQ ID NO: 3; or
(15) the antigen-binding domain specifically binding to CD3 comprises the following variable region amino acid sequences or variants thereof:
(i) a heavy chain variable region amino acid sequence set forth in SEQ ID NO: 16, and
(ii) a light chain variable region amino acid sequence set forth in SEQ ID NO: 17; and
the antigen-binding domain specifically binding to CLDN18.2 comprises the following variable region amino acid sequences or variants thereof:
(i) a heavy chain variable region amino acid sequence set forth in SEQ ID NO: 6, and
(ii) a light chain variable region amino acid sequence set forth in SEQ ID NO: 3;
wherein the variants differ from the variable region amino acid sequences by 3, 2 or 1 amino acids or have at least 80%, 85%, 90%, 91%, 92%, 93%, 94%, 95%, 96%, 97%, 98% or 99% identity thereto.
8. The bispecific antibody according to any one of items 1 to 7, wherein the antigen-binding domain specifically binding to CLDN18.2 is in the form of an Fab fragment and the antigen-binding domain specifically binding to CD3 is in the form of an scFv, preferably, the bispecific antibody comprising:
a) a first monomer, comprising a first heavy chain,
wherein the first heavy chain comprises:
1) a first heavy chain variable region;
2) a first heavy chain constant region, comprising a first CH1 domain and a first Fc domain (wherein, preferably the first CH1 domain and the first Fc domain are linked by hinge region 1, and preferably the first Fc domain is selected from an IgG1 subtype, an IgG2 subtype, an IgG3 subtype and an IgG4 subtype);
3) an antigen-binding domain specifically binding to CD3 (preferably human CD3), wherein a heavy chain variable region and a light chain variable region in the antigen-binding domain are linked by a linker peptide and positions of the two are interchangeable, wherein the antigen-binding domain specifically binding to CD3 is as defined in any one of items 1 to 7, wherein
i) the antigen-binding domain is linked to a C-terminus of the first CH1 domain and an N-terminus of the first Fc domain by a linker peptide, preferably by a linker peptide and a hinge region (e.g., hinge region 3), and preferably the antigen-binding domain is linked to the first Fc domain by a linker peptide and a hinge region (e.g., hinge region 2), or
ii) the antigen-binding domain is linked to a C-terminus of the first Fc domain by a linker peptide, preferably by a linker peptide and/or a hinge region (e.g., hinge region 1, 2 or 3), or
iii) the antigen-binding domain is linked to an N-terminus of the first heavy chain variable region by a linker peptide, preferably by a linker peptide and/or a hinge region (e.g., hinge region 1, 2 or 3);
b) a second monomer, comprising a second heavy chain,
wherein the second heavy chain comprises: a second heavy chain variable region and a second heavy chain constant region, the second heavy chain constant region comprising a second CH1 domain and a second Fc domain (preferably the second CH1 domain and the second Fc domain are linked by a hinge region (e.g., hinge region 1), and preferably the second Fc domain is selected from an IgG1 subtype, an IgG2 subtype, an IgG3 subtype and an IgG4 subtype),
c) a first light chain assembled with the first heavy chain and comprising a first light chain variable region and a first light chain constant region;
d) a second light chain assembled with the second heavy chain and comprising a second light chain variable region and a second light chain constant region;
wherein the first light chain constant region is identical to or different from the second light chain constant regions, the first CH1 domain is identical to or different from the second CH1 domain, and the first Fc domain is identical to or different from the second Fc domain;
wherein the first heavy chain variable region and the first light chain variable region form a first antigen-binding domain, the second heavy chain variable region and the second light chain variable region form a second antigen-binding domain, and a sequence of the first antigen-binding domain and the second antigen-binding domain is a sequence of the antigen-binding domain specifically binding to CLDN18.2 as defined in any one of items 1 to 7, and the first antigen-binding domain and the second antigen-binding domain have identical or different sequences.
9. The bispecific antibody according to any one of items 1 to 7, wherein the antigen-binding domain specifically binding to CLDN18.2 is in the form of an Fab fragment and the antigen-binding domain specifically binding to CD3 is in the form of an scFv, preferably, the bispecific antibody comprising:
a) a first monomer, comprising a heavy chain,
wherein the heavy chain comprises:
1) a first heavy chain variable region;
2) a heavy chain constant region comprising a CH1 domain and a first Fc domain (preferably the CH1 domain and the first Fc domain are linked by a hinge region (e.g., hinge region 1), and preferably the first Fc domain is selected from an IgG1 subtype, an IgG2 subtype, an IgG3 subtype and an IgG4 subtype);
b) a second monomer, comprising an antigen-binding domain specifically binding to CD3 (preferably human CD3), wherein the antigen-binding domain is linked to an N-terminus of a second Fc domain (preferably the second Fc domain is selected from an IgG1 subtype, an IgG2 subtype, an IgG3 subtype and an IgG4 subtype) by a linker peptide (preferably by a linker peptide and/or a hinge region (e.g., hinge region 1, 2 or 3)), wherein a heavy chain variable region and a light chain variable region in the antigen-binding domain specifically binding to CD3 are linked by a linker peptide and positions of the two are interchangeable, wherein the antigen-binding domain specifically binding to CD3 is as defined in any one of items 1 to 7,
c) a light chain assembled with the heavy chain and comprising a first light chain variable region and a light chain constant region;
wherein the first heavy chain variable region and the first light chain variable region form the antigen-binding domain specifically binding to CLDN18.2 as defined in any one of items 1 to 7.
10. The bispecific antibody according to item 9, wherein the antigen-binding domain specifically binding to CLDN18.2 is selected from the group consisting of:
1) an antigen-binding domain specifically binding to CLDN18.2 comprising the following CDRs (or variants thereof) or variable regions (or variants thereof):
(i) CDRH1, CDRH2 and CDRH3 comprised in a heavy chain variable region set forth in SEQ ID NO: 5, or a heavy chain variable region set forth in SEQ ID NO: 5, and
(ii) CDRL1, CDRL2 and CDRL3 comprised in a light chain variable region set forth in SEQ ID NO: 2, or a light chain variable region set forth in SEQ ID NO: 2; or
2) an antigen-binding domain specifically binding to CLDN18.2 comprising the following CDRs (or variants thereof) or variable regions (or variants thereof):
(i) CDRH1, CDRH2 and CDRH3 comprised in a heavy chain variable region set forth in SEQ ID NO: 6, or a heavy chain variable region set forth in SEQ ID NO: 6, and
(ii) CDRL1, CDRL2 and CDRL3 comprised in a light chain variable region set forth in SEQ ID NO: 3, or a light chain variable region set forth in SEQ ID NO: 3,
wherein the variants differ from the CDRs or the variable regions by 3, 2 or 1 amino acids or have at least 80%, 85%, 90%, 91%, 92%, 93%, 94%, 95%, 96%, 97%, 98% or 99% identity thereto.
11. The bispecific antibody according to item 8 or 9, wherein the light chain constant region CL has a sequence set forth in SEQ ID NO: 18, 98, 99 or 100, and the CH1 domain has a sequence set forth in SEQ ID NO: 19, 101, 102 or 103.
12. The bispecific antibody according to item 8 or 9, wherein the first Fc domain or the second Fc domain has identical or different sequence, preferably selected from SEQ ID NO: 20, 21, 22, 23, 24, 25 or 26.
13. The bispecific antibody according to item 8 or 9, wherein the hinge region 1 has a sequence set forth in SEQ ID NO: 27, the hinge region 2 has a sequence set forth in SEQ ID NO: 28, and/or the hinge region 3 has a sequence set forth in SEQ ID NO: 29.
14. The bispecific antibody according to item 8 or 9, wherein the linker peptide has a sequence selected from sequences set forth in SEQ ID NOs: 8, 30, 31 and 32.
15. A nucleic acid composition, comprising a nucleic acid sequence encoding the bispecific antibody according to any one of items 1 to 14, wherein preferably,
the nucleic acid composition comprises:
a) a first expression vector comprising a first nucleic acid encoding the antigen-binding domain specifically binding to CD3 as defined in item 5;
b) a second expression vector comprising a second nucleic acid encoding the antigen-binding domain specifically binding to CLDN18.2 as defined in item 5; or
the nucleic acid composition comprises:
a) a first expression vector comprising a first nucleic acid encoding the antigen-binding domain specifically binding to CD3 as defined in item 6;
b) a second expression vector comprising a second nucleic acid encoding the antigen-binding domain specifically binding to CLDN18.2 as defined in item 6; or
the nucleic acid composition comprises:
a) a first expression vector comprising a first nucleic acid encoding the antigen-binding domain specifically binding to CD3 as defined in item 7;
b) a second expression vector comprising a second nucleic acid encoding the antigen-binding domain specifically binding to CLDN18.2 as defined in item 7; or
the nucleic acid composition comprises:
a) a first expression vector comprising encoding the first monomer as defined in item 8;
b) a second expression vector comprising encoding the second monomer as defined in item 8;
c) a third expression vector comprising encoding the first light chain as defined in item 8; and
d) a fourth expression vector comprising encoding the second light chain as defined in item 8; or
the nucleic acid composition comprises:
a) a first expression vector comprising encoding the first monomer as defined in item 9;
b) a second expression vector comprising encoding the second monomer as defined in item 9, and
c) a third expression vector comprising encoding the light chain as defined in item 9.
16. An expression vector, comprising the nucleic acid composition according to item 15.
17. A host cell, comprising the expression vector according to item 16.
18. A pharmaceutical composition, comprising the bispecific antibody according to any one of items 1 to 14 and a pharmaceutical carrier, and optionally, a drug (e.g., a small molecule drug or a macromolecular drug) for the treatment of cancer (e.g., gastric cancer, pancreatic cancer, ovarian cancer, esophageal cancer or non-small cell lung cancer).
19. A kit, comprising the bispecific antibody according to any one of items 1 to 14, and optionally, a drug (e.g., a small molecule drug or a macromolecular drug) for the treatment of cancer (e.g., gastric cancer, pancreatic cancer, ovarian cancer, esophageal cancer or non-small cell lung cancer).
20. Use of the bispecific antibody according to any one of items 1 to 14 in the treatment of, or in the preparation of a medicament for treating, cancer, e.g., gastric cancer, pancreatic cancer, ovarian cancer, esophageal cancer, or non-small cell lung cancer.
21. A method for treating cancer, comprising administering to a subject a therapeutically effective amount of the bispecific antibody according to any one of items 1 to 14, wherein the cancer is, for example, gastric cancer, pancreatic cancer, ovarian cancer, esophageal cancer or non-small cell lung cancer.
22. A bispecific antibody conjugate, comprising the bispecific antibody according to any one of items 1 to 14 and a further substance, wherein the further substance is selected from one or more of a therapeutic agent, a prodrug, a peptide, a protein, an enzyme, a virus, a lipid, a biological response modifier, a pharmaceutical agent and PEG, and preferably the therapeutic agent comprises a detectable label, e.g., a radioactive label, an immunomodulator, a hormone, an enzyme, an oligonucleotide, a photoactive therapeutic or diagnostic agent, a cytotoxic agent such as a drug or a toxin, an ultrasound enhancing agent and a nonradioactive label.

In some embodiments, a VH and/or VL of the antigen-binding domain comprises framework regions (FRs) from human, rabbit or mouse immunoglobulins.

In some embodiments, the Fc domain (e.g., a first Fc domain and/or a second Fc structure) is wild-type or mutant Fc domain. In some embodiments, the Fc domain (e.g., a first Fc domain and/or a second Fc domain) comprises one or more amino acid substitutions that form an ionic bond and/or a pestle-mortar structure pairing between a heavy chain and an Fc fragment as compared to a wild-type antibody Fc fragment. In some embodiments, amino acid mutation and engineering is performed on the Fc domain (e.g., a first Fc domain and/or a second Fc domain) to make each domain less prone to form a homodimer and more prone to form a heterodimer. In some embodiments, the Fc domain contains or does not contain a hinge region.

The bispecific antibody of the present invention specifically binds to CLDN18.2 antigen and not to CLDN18.1 antigen. In some embodiments, the CLDN18.2 antigen comprises a human CLDN18.2 antigen, a murine CLDN18.2 antigen, or a monkey CLDN18.2 antigen. In some embodiments, the murine CLDN18.2 antigen comprises a murine CLDN18A2.1 antigen and a murine CLDN18A2.2 antigen. In some embodiments, the CLDN18.2 antigen sequence includes a sequence set forth in SEQ ID NO: 40, 43 or 44.

The CD3 antigen bound by the bispecific antibody of the present invention includes a human CD3 antigen, a murine CD3 antigen, a rabbit CD3 antigen, a goat CD3 antigen, or a monkey CD3 antigen.

In some embodiments, bispecific antibodies Y1, Y2, Y4, Y6, Y7, Y8, Y17, Y18 and SY1 have YBODY structures. In some embodiments, bispecific antibodies CS1, CS2, CS3, CS4, CS5, CS6, CS7, CS8, CS9, CS10, CS11 and SCS1 have CSBODY structures.

Another aspect of the present invention also relates to an antigen-binding molecule specifically binding to CD3 (preferably human CD3), wherein the antigen-binding molecule comprises:

(i) a heavy chain variable region set forth in SEQ ID NO: 12, or
an amino acid sequence having at least 90%, 91%, 92%, 93%, 94%, 95%, 96%, 97%, 98% or 99% identity to an amino acid sequence set forth in SEQ ID NO:12, or
an amino acid sequence differing from a heavy chain variable region set forth in SEQ ID NO: 12 by 3, 2 or 1 amino acids; and
(ii) a light chain variable region set forth in SEQ ID NO: 13, or
an amino acid sequence having at least 90%, 91%, 92%, 93%, 94%, 95%, 96%, 97%, 98% or 99% identity to an amino acid sequence set forth in SEQ ID NO: 13, or
an amino acid sequence differing from a light chain variable region set forth in SEQ ID NO: 13 by 3, 2 or 1 amino acids.

In some embodiments, the antigen-binding molecule specifically binding to CD3 is a single-chain antibody, a diabody or an antigen-binding fragment.

Another aspect of the present invention further relates to a bispecific antibody comprising the antigen-binding molecule specifically binding to CD3 described above and an antigen-binding domain specifically binding to another antigen (e.g., EGFR, Her2, EpCAM, CD20, CD30, CD33, CD38, BCMA, PD-L1, PD-1, TIM-3, TGF-β, LAG-3, VISTA, CTLA-4, OX40, BTLA, 4-1BB, CD96, CD27, CD28, CD40, LAIR1, CD160, 2B4, TGF-R, KIR, ICOS, GITR, BAFFR, HVEM, CD7, LIGHT, NKp80, B7-H3, SLAMF7, Claudin18.2, CEA, CD47, CD52, CD133, CEA, gpA33, mucin, TAG-72, CIX, PSMA, folate-binding protein, GD2, GD3, GM2, VEGF, VEGFR, EGFR, integrin, αVβ3, α5β1, ERBB2, ERBB3, MET, IGF1R, EPHA3, TRAILR1, TRAILR2, RANKL, FAP or Tenascin).

Yet another aspect of the present invention further relates to polynucleotides encoding the antigen-binding molecule specifically binding to CD3 described above or the bispecific antibody described above comprising the same.

It is to be understood that within the scope of the present invention, the above various technical features of the present invention and the technical features specifically described hereinafter (as in the examples) may be combined with each other to constitute a new or preferred technical scheme. Due to limited space, such schemes are not described herein.

The terms referred to in the present invention have the conventional meanings understood by those skilled in the art. Where a term has two or more definitions as used and/or acceptable in the art, the definitions of the terms used herein are intended to include all meanings.

It will be understood by those of ordinary skill in the art that the CDR regions of an antibody are responsible for the binding specificity of the antibody for an antigen. Given the known sequences of the heavy and light chain variable regions of an antibody, there are several methods for determining the CDR regions of the antibody, including the Kabat, Chothia and AbM numbering systems.

Specifically, the AbM numbering system: the AbM method for defining CDRs was derived from Martin's related research (Martin A C R, Cheetham J C, Rees A R (1989) Modelling antibody hypervariableloops: A combined algorithm. Proc Natl Acad Sci USA 86: 9268-9272), and this method integrates partial definitions of Kabat and Chothia method.

The Kabat numbering system: an immunoglobulin alignment and numbering system was proposed by Elvin A. Kabat (see, e.g., Kabat et al., Sequences of Proteins of Immunological Interest, 5th Ed. Public Health Service, National Institutes of Health, Bethesda, Md., 1991).

The Chothia numbering system: an immunoglobulin numbering system was proposed by Chothia et al., which was a classical rule for identifying the boundaries of CDR regions based on the position of structural loop regions (see, e.g., Chothia & Lesk (1987) J. Mol. Biol. 196:901-917; Chothia et al., (1989) Nature 342:878-883).

The IMGT numbering system: a numbering system based on The international ImMunoGeneTics Information System® (IMGT) proposed by Lefranc et al. may refer to Lefranc et al., Dev. Comparat. Immunol. 27:55-77, 2003.

For example, sequences of a heavy chain variable region and a light chain variable region of an antibody can be determined at the website http://abysis.org/.

However, the application of all the definitions of CDRs for an antibody or its variant shall fall within the scope of the terms defined and used herein. If an amino acid sequence of the variable region of the antibody is known, those skilled in the art can generally determine which residues comprise a particular CDR, without relying on any experimental data beyond the sequence itself. Suitable amino acid residues of the CDRs as defined by the Kabat and Chothia CDR numbering systems are listed below for comparison. The exact number of residues comprising a particular CDR will vary with the sequence and size of that CDR.

| | Kabat | Chothia |
|---|---|---|
| CDR-H1 | 31-35 | 26-32 |
| CDR-H2 | 50-65 | 52-58 |
| CDR-H3 | 95-102 | 95-102 |
| CDR-L1 | 24-34 | 26-32 |
| CDR-L2 | 50-56 | 50-52 |
| CDR-L3 | 89-97 | 91-96 |

In addition to the above table, the Kabat numbering system describes the CDRs as follows: CDR-H1 begins at about the 31^{st} amino acid (i.e., about 9 residues after a first cysteine residue), comprises about 5-7 amino acids, and ends at the next tryptophan residue. CDR-H2 begins at the 15^{th} residue after the terminus of CDR-H1, comprises about 16-19 amino acids, and ends at the next arginine or lysine residue. CDR-H3 begins at about the 33^{rd} amino acid residue after the terminus of CDR-H2, comprises 3-25 amino acids; and ends at the sequence W-G-X-G, wherein X is any amino acid. CDR-L1 begins at about the 24^{th} residue (i.e., after cysteine residue), comprises about 10-17 residues, and ends at the next tryptophan residue. CDR-L2 begins at about the 16^{th} residues after the terminus of CDR-L1 and comprises about 7 residues. CDR-L3 begins at about the 33^{rd} residue (i.e., after cysteine residue) after the terminus of CDR-L2, comprises about 7-11 residues, and ends at the sequence F or W-G-X-G, wherein X is any amino acid.

Embodiments of the present application provide a plurality of bispecific antibodies comprising two or more different or identical antigen-binding domains. An antigen-binding domain binding to an antigen is an Fab, or an ScFv, or non-covalent pairing (Fv) between a heavy chain variable region (VH) and a light chain variable region (VL). Any of the above antibodies or polypeptides may also include additional polypeptides, e.g., a signal peptide at an N-terminus of the antibody, which is used to direct secretion, or other heterologous polypeptides as described herein.

For the purpose of comparing two or more amino acid sequences, the percentage of “sequence homology” (also referred to herein as “amino acid homology”) between a first amino acid sequence and a second amino acid sequence can be calculated by dividing [the number of amino acid residues in a first amino acid sequence that are identical to the amino acid residues at the corresponding positions in a second amino acid sequence] by [the total number of amino acid residues in the first amino acid sequence] and multiplying by [100%], wherein each deletion, insertion, substitution or addition of an amino acid residue in the second amino acid sequence, as compared to the first amino acid sequence, is considered a difference in a single amino acid residue (position), i.e., an “amino acid difference” as defined herein.

Alternatively, the degree of sequence identity between two amino acid sequences can be calculated using known computer algorithms, such as NCBI embryonic cell v2.0. Some other techniques, computer algorithms and arrangements for determining the degree of sequence identity are described, for example, in WO 04/037999, EP 0 967 284, EP 1 085 089, WO 00/55318, WO 00/78972, WO 98/49185 and GB 2 357 768-A.

Typically, for the purpose of determining the percentage of “sequence identity” between two amino acid sequences according to the calculation methods set forth above, an amino acid sequence with the greatest number of amino acid residues is considered a “first” amino acid sequence, and the other amino acid sequence is considered a “second” amino acid sequence.

Furthermore, when determining the degree of sequence identity between two amino acid sequences, those skilled in the art may consider so-called “conservative” amino acid substitutions, which may generally be described as amino acid substitutions in which an amino acid residue is substituted with another amino acid residue having a similar chemical structure with little or no effect on the function, activity or other biological properties of the polypeptide. Such conservative amino acid substitutions are well known in the art, for example, from WO 04/037999, GB-A-3357 768, WO 98/49185, WO 00/46383 and WO 01/09300; and such substitutive (preferred) types and/or combinations may be selected based on the relevant teachings of WO 04/037999 and WO 98/49185 and other references cited therein.

A “conservative amino acid substitution” is one in which an amino acid residue is substituted with an amino acid residue having a similar side chain. Families of amino acid residues having similar side chains have been defined in the art, including basic side chains (e.g., lysine, arginine and histidine), acidic side chains (e.g., aspartic acid and glutamic acid), uncharged polar side chains (e.g., glycine, asparagine, glutamine, serine, threonine, tyrosine and cysteine), non-polar side chains (e.g., alanine, valine, leucine, isoleucine, proline, phenylalanine, methionine and tryptophan), (3-branched side chains (e.g., threonine, valine and isoleucine) and aromatic side chains (e.g., tyrosine, phenylalanine, tryptophan and histidine). Thus, non-essential amino acid residues of an immunoglobulin polypeptide are preferably substituted with other amino acid residues from the same side chain family. In other embodiments, a string of amino acids may be substituted with a structurally similar string of amino acids that differ in sequence and/or composition of the side chain family.

Non-limiting examples of conservative amino acid substitutions are provided in the following table, where a similarity score of 0 or higher indicates that there is a conservative substitution between the two amino acids.

| | C | G | P | S | A | T | D | E | N | Q | H | K | R | V | M | I | L | F | Y | W |
|---|---|---|---|---|---|---|---|---|---|---|---|---|---|---|---|---|---|---|---|---|
| W | −8 | −7 | −6 | −2 | −6 | −5 | −7 | −7 | −4 | −5 | −3 | −3 | 2 | −6 | −4 | −5 | −2 | 0 | 0 | 17 |
| Y | 0 | −5 | −5 | −3 | −3 | −3 | −4 | −4 | −2 | −4 | 0 | −4 | −5 | −2 | −2 | −1 | −1 | 7 | 10 | |
| F | −4 | −5 | −5 | −3 | −4 | −3 | −6 | −5 | −4 | −5 | −2 | −5 | −4 | −1 | 0 | 1 | 2 | 9 | | |
| L | −6 | −4 | −3 | −3 | −2 | −2 | −4 | −3 | −3 | −2 | −2 | −3 | −3 | 2 | 4 | 2 | 6 | | | |
| I | −2 | −3 | −2 | −1 | −1 | 0 | −2 | −2 | −2 | −2 | −2 | −2 | −2 | 4 | 2 | 5 | | | | |
| M | −5 | −3 | −2 | −2 | −1 | −1 | −3 | −2 | 0 | −1 | −2 | 0 | 0 | 2 | 6 | | | | | |
| V | −2 | −1 | −1 | −1 | 0 | 0 | −2 | −2 | −2 | −2 | −2 | −2 | −2 | 4 | | | | | | |
| R | −4 | −3 | 0 | 0 | −2 | −1 | −1 | −1 | 0 | 1 | 2 | 3 | 6 | | | | | | | |
| K | −5 | −2 | −1 | 0 | −1 | 0 | 0 | 0 | 1 | 1 | 0 | 5 | | | | | | | | |
| H | −3 | −2 | 0 | −1 | −1 | −1 | 1 | 1 | 2 | 3 | 6 | | | | | | | | | |
| Q | −5 | −1 | 0 | −1 | 0 | −1 | 2 | 2 | 1 | 4 | | | | | | | | | | |
| N | −4 | 0 | −1 | 1 | 0 | 0 | 2 | 1 | 2 | | | | | | | | | | | |
| E | −5 | 0 | −1 | 0 | 0 | 0 | 3 | 4 | | | | | | | | | | | | |
| D | −5 | 1 | −1 | 0 | 0 | 0 | 4 | | | | | | | | | | | | | |
| T | −2 | 0 | 0 | 1 | 1 | 3 | | | | | | | | | | | | | | |
| A | −2 | 1 | 1 | 1 | 2 | | | | | | | | | | | | | | | |
| S | 0 | 1 | 1 | 1 | | | | | | | | | | | | | | | | |
| P | −3 | −1 | 6 | | | | | | | | | | | | | | | | | |
| G | −3 | 5 | | | | | | | | | | | | | | | | | | |
| C | 12 | | | | | | | | | | | | | | | | | | | |

In some embodiments, the conservative substitution is preferably a substitution in which one amino acid within the following groups (a)-(e) is substituted with another amino acid residue within the same group: (a) small aliphatic, non-polar or weakly polar residues: Ala, Ser, Thr, Pro and Gly, (b) polar, negatively charged residues and (uncharged) amides thereof: Asp, Asn, Glu and Gln, (c) polar, positively charged residues: His, Arg and Lys, (d) bulky aliphatic, nonpolar residues: Met, Leu, Ile, Val and Cys, and (e) aromatic residues: Phe, Tyr and Trp.

Particularly preferred conservative substitutions are as follows: Ala is substituted with Gly or Ser; Arg is substituted with Lys; Asn is substituted with Gln or His; Asp is substituted with Glu; Cys is substituted with Ser; Gln is substituted with Asn; Glu is substituted with Asp; Gly is substituted with Ala or Pro; His is substituted with Asn or Gln; Ile is substituted with Leu or Val; Leu is substituted with Ile or Val; Lys is substituted with Arg, Gln or Glu; Met is substituted with Leu, Tyr or Ile; Phe is substituted with Met, Leu or Tyr; Ser is substituted with Thr; Thr is substituted with Ser; Trp is substituted with Tyr; Tyr is substituted with Trp; and/or Phe is substituted with Val, Ile or Leu.

In some embodiments, the bispecific antibody may be conjugated with a therapeutic agent, a prodrug, a peptide, a protein, an enzyme, a virus, a lipid, a biological response modifier, a pharmaceutical agent or PEG. The bispecific antibody can be linked or fused to a therapeutic agent, and the therapeutic agent can include a detectable label such as a radioactive label, an immunomodulator, a hormone, an enzyme, an oligonucleotide, a photoactive therapeutic or diagnostic agent, a cytotoxic agent that can be a drug or a toxin, an ultrasound enhancing agent, a nonradioactive label, a combination thereof and other such ingredients known in the art.

Specifically, the sequences of the present invention are shown below.

1. CDR sequences in the anti-CLDN18.2 antibody:

### 175D10:

SEQ ID NO: 47
VH-CDR1 GYTFTSYW
SEQ ID NO: 48
VH-CDR2 IYPSDSYT
SEQ ID NO: 49
VH-CDR3 TRSWRGNSFDY
SEQ ID NO: 50
VL-CDR1 QSLLNSGNQKNY
SEQ ID NO: 51
VL-CDR2 WAS
SEQ ID NO: 52
VL-CDR3 QNDYSYPF

1E9.2 (CDRs determined according to IMGT):

SEQ ID NO: 53
VH-CDR1 GFSFSNSA
SEQ ID NO: 54
VH-CDR2 ISSGDSYT
SEQ ID NO: 55
VH-CDR3 ARQGYGNALDY
SEQ ID NO: 56
VL-CDR1 QSLLNSGNQKNY
SEQ ID NO: 57
VL-CDR2 WS
SEQ ID NO: 58
VL-CDR3 QNDYYYPLT

1E9.2 (CDRs determined according to AbM):

SEQ ID NO: 59
VH-CDR1 GFSFSNSAMS
SEQ ID NO: 60
VH-CDR2 TISSGDSYTY
SEQ ID NO: 61
VH-CDR3 QGYGNALDY
SEQ ID NO: 62
VL-CDR1 KSSQSLLNSGNQKNYLT
SEQ ID NO: 63
VL-CDR2 WSSTRES
SEQ ID NO: 58
VL-CDR3 QNDYYYPLT

1E9.2 (CDRs determined according to Kabat):

SEQ ID NO:64
VH-CDR1 NSAMS
SEQ ID NO: 65
VH-CDR2 TISSGDSYTYYADSVKG
SEQ ID NO: 61
VH-CDR3 QGYGNALDY
SEQ ID NO: 62
VL-CDR1 KSSQSLLNSGNQKNYLT
SEQ ID NO: 63
VL-CDR2 WSSTRES
SEQ ID NO: 58
VL-CDR3 QNDYYYPLT

2C6.9 (CDRs determined according to IMGT):

SEQ ID NO: 66
VH-CDR1 GFSLTRYG
SEQ ID NO: 67
VH-CDR2 IWGEGNT
SEQ ID NO: 68
VH-CDR3 ARVNFGNALDY
SEQ ID NO: 69
VL-CDR1 QSLLNSGNQKNY
SEQ ID NO: 70
VL-CDR2 WA
SEQ ID NO: 71
VL-CDR3 QNDFIFPLT

2C6.9 (CDRs determined according to AbM):

SEQ ID NO: 72
VH-CDR1 GFSLTRYGVS
SEQ ID NO: 73
VH-CDR2 VIWGEGNTN
SEQ ID NO: 74
VH-CDR3 VNFGNALDY
SEQ ID NO: 75
VL-CDR1 KSSQSLLNSGNQKNYLT
SEQ ID NO: 76
VL-CDR2 WASTRDS
SEQ ID NO: 71
VL-CDR3 QNDFIFPLT

2C6.9 (CDRs determined according to Kabat):

SEQ ID NO: 77
VH-CDR1 RYGVS
SEQ ID NO: 78
VH-CDR2 VIWGEGNTNYNPSLKS
SEQ ID NO: 74
VH-CDR3 VNFGNALDY
SEQ ID NO: 75
VL-CDR1 KSSQSLLNSGNQKNYLT
SEQ ID NO: 76
VL-CDR2 WASTRDS
SEQ ID NO: 71
VL-CDR3 QNDFIFPLT

2. CDR sequences in the anti-CD3 scFv:
2a5ga (CDRs determined according to Kabat):

SEQ ID NO: 79
VH-CDR1 TYAMN
SEQ ID NO: 80
VH-CDR2 RIRSKYNNYATYYADSVKD
SEQ ID NO: 81
VH-CDR3 HGNFGNSYVSWAAY
SEQ ID NO: 82
VL-CDR1 RSSTGAVTTSNYAN
SEQ ID NO: 83
VL-CDR2 GTNKRAP
SEQ ID NO: 84
VL-CDR3 ALWYSNLWV

2a5 (CDRs determined according to Kabat):

SEQ ID NO: 79
VH-CDR1 TYAMN
SEQ ID NO: 80
VH-CDR2 RIRSKYNNYATYYADSVKD
SEQ ID NO: 85
VH-CDR3 HGNFGNSYVSWFAY
SEQ ID NO: 82
VL-CDR1 RSSTGAVTTSNYAN
SEQ ID NO: 83
VL-CDR2 GTNKRAP
SEQ ID NO: 84
VL-CDR3 ALWYSNLWV

Dib-2 (CDRs determined according to Kabat):

SEQ ID NO: 79
VH-CDR1 TYAMN
SEQ ID NO: 80
VH-CDR2 RIRSKYNNYATYYADSVKD
SEQ ID NO: 85
VH-CDR3 HGNFGNSYVSWFAY
SEQ ID NO: 82
VL-CDR1 RSSTGAVTTSNYAN
SEQ ID NO: 83
VL-CDR2 GTNKRAP
SEQ ID NO: 84
VL-CDR3 ALWYSNLWV

L2K (CDRs determined according to Kabat):

SEQ ID NO: 86
VH-CDR1 RYTMH
SEQ ID NO: 87
VH-CDR2 YINPSRGYTNYNQKFKD
SEQ ID NO: 88
VH-CDR3 YYDDHYCLDY
SEQ ID NO: 89
VL-CDR1 RASSSVSYMN
SEQ ID NO: 90
VL-CDR2 DTSKVAS
SEQ ID NO: 91
VL-CDR3 QQWSSNPLT

2C11 (CDRs determined according to Kabat):

SEQ ID NO: 92
VH-CDR1 GYGMH
SEQ ID NO: 93
VH-CDR2 YITSSSINIKYADAVKG
SEQ ID NO: 94
VH-CDR3 FDWDKNY
SEQ ID NO: 95
VL-CDR1 QASQDISNYLN
SEQ ID NO: 96
VL-CDR2 YTNKLAD
SEQ ID NO: 97
VL-CDR3 QQYYNYPWT

3. Variable region sequences in the anti-CLDN18.2 antibody:

| Antibody | | | |
|---|---|---|---|
| No. | | | |
| (sequence | | | SEQ ID |
| source) | Amino acid sequence | | NO: |
| 175D10 | VL | DIVMTQSPSSLTVTAGEKVTMSCKSSQSLLNSGNQKNYLTWYQQKPG | 1 |
| | | QPPKLLIYWASTRESGVPDRFTGSGSGTDFTLTISSVQAEDLAVYYCQN | |
| | | DYSYPFTFGSGTKLEIK | |
| | VH | QVQLQQPGAELVRPGASVKLSCKASGYTFTSYWINWVKQRPGQGLE | 4 |
| | | WIGNIYPSDSYTNYNQKFKDKATLTVDKSSSTAYMQLSSPTSEDSAVY | |
| | | YCTRSWRGNSFDYWGQGTTLTVSS | |
| 1E9.2 | VL | DIVMTQSPDSLAVSLGERATINCKSSQSLLNSGNQKNYLTWYQQKPGQ | 2 |
| | | PPKLLIYWSSTRESGVPDRFSGSGSGTDFTLTISSLQAEDFAVYYCQND | |
| | | YYYPLTFGGGTKVEIK | |
| | VH | EVQLVESGGGLVKPGGSLRLSCAASGFSFSNSAMSWVRQAPGKGLEW | 5 |
| | | VSTISSGDSYTYYADSVKGRFTISRDNAKNSLYLQMNSLRAEDTAVYY | |
| | | CARQGYGNALDYWGQGTLVTVSS | |
| 2C6.9 | VL | DIVMTQSPDSLAVSLGERATINCKSSQSLLNSGNQKNYLTWYQQKPGQ | 3 |
| | | PPKLLIYWASTRDSGVPDRFSGSGSGTDFTLTISSLQAEDVAVYYCQND | |
| | | FIFPLTFGGGTKVEIK | |
| | VH | QVQLQESGPGLVKPSETLSLTCTVSGFSLTRYGVSWIRQPPGKGLEWIG | 6 |
| | | VIWGEGNTNYNPSLKSRVTISKDSSKSQVSLKLSSVTAADTAVYYCAR | |
| | | VNFGNALDYWGQGTLVTVSS | |

4. Variable region sequences in the anti-CD3 scFv:

| Antibody No. | | | |
|---|---|---|---|
| (sequence | | | SEQ ID |
| source) | Amino acid sequence of variable region | | NO: |
| 2a5ga | VH | QVQLVESGGGVVQPGRSLRLSCAASGFTFSTYAMNWVRQAPGKG | 7 |
| (anti-human | | LEWVARIRSKYNNYATYYADSVKDRFTISRDDSKNTLYLQMNSLR | |
| CD3) | | AEDTAVYYCARHGNFGNSYVSWAAYWGQGTLVTVSS | |
| | VL | QTVVTQEPSLTVSPGGTVTLTCRSSTGAVTTSNYANWFQQKPGQAP | 9 |
| | | RGLIGGTNKRAPGVPARFSGSLLGGKAALTLSGVQPEDEAEYYCA | |
| | | LWYSNLWVFGGGTKVEIK | |
| 2a5 | VH | QVQLVESGGGVVQPGRSLRLSCAASGFTFSTYAMNWVRQAPGKG | 10 |
| (anti-human | | LEWVARIRSKYNNYATYYADSVKDRFTISRDDSKNTLYLQMNSLR | |
| CD3) | | AEDTAVYYCARHGNFGNSYVSWFAYWGQGTLVTVSS | |
| | VL | QTVVTQEPSLTVSPGGTVTLTCRSSTGAVTTSNYANWVQQKPGQA | 11 |
| | | PRGLIGGTNKRAPGVPARFSGSLLGGKAALTLSGVQPEDEAEYYC | |
| | | ALWYSNLWVFGGGTKVEIK | |
| Di6-2 | VH | QVQLVESGGGVVQPGRSLRLSCAASGFTFSTYAMNWVRQAPGKG | 12 |
| (anti-human | | LEWVGRIRSKYNNYATYYADSVKDRFTISRDDSKNTLYLQMNSLR | |
| CD3) | | GEDTAVYYCARHGNFGNSYVSWFAYWGQGTLVTVSS | |
| | VL | QTVVTQEPSLTVSPGGTVTLTCRSSTGAVTTSNYANWVQQKPGQA | 13 |
| | | PRGLIGGTNKRAPGVPARFSGSLLGGKAALTLSGVQPEDEAEYTCA | |
| | | LWYSNLWVFGGGTKVEIK | |
| L2K | VH | DIKLQQSGAELARPGASVKMSCKTSGYTFTRYTMHWVKQRPGQG | 14 |
| (anti-human | | LEWIGYINPSRGYTNYNQKFKDKATLTTDKSSSTAYMQLSSLTSED | |
| CD3) | | SAVYYCARYYDDHYCLDYWGQGTTLTVSS | |
| | VL | DIQLTQSPAIMSASPGEKVTMTCRASSSVSYMNWYQQKSGTSPKR | 15 |
| | | WIYDTSKVASGVPYRFSGSGSGTSYSLTISSMEAEDAATYYCQQWS | |
| | | SNPLTFGAGTKLELK | |
| 2C11 | VH | EVQLVESGGGLVQPGKSLKLSCEASGFTFSGYGMHWVRQAPGRG | 16 |
| (anti-mouse | | LESVAYITSSSINIKYADAVKGRFTVSRDNAKNLLFLQMNILKSEDT | |
| CD3) | | AMYYCARFDWDKNYWGQGTMVTVSS | |
| | VL | DIQMTQSPSSLPASLGDRVTINCQASQDISNYLNWYQQKPGKAPKL | 17 |
| | | LIYYTNKLADGVPSRFSGSGSGRDSSFTISSLESEDIGSYYCQQYYN | |
| | | YPWTFGPGTKLEIK | |

5. CH1 and CL sequences:

| | | SEQ ID |
|---|---|---|
| Domain | Amino acid sequence | NO: |
| CL | RTVAAPSVFIFPPSDEQLKSGTASVVCLLNNFYPREAKVQWKVDNALQSGNSQ | 18 |
| | ESVTEQDSKDSTYSLSSTLTLSKADYEKHKVYACEVTHQGLSSPVTKSFNRGEC | |
| | GQPKANPTVTLFPPSSEELQANKATLVCLISDFYPGAVTVAWKADGSPVKAGV | 98 |
| | ETTKPSKQSNNKYAASSYLSLTPEQWKSHRSYSCQVTHEGSTVEKTVAPTECS | |
| | GQPKAAPSVTLFPPSSEELQANKATLVCLISDFYPGAVTVAWKADSSPVKAGVE | 99 |
| | TTTPSKQSNNKYAASSYLSLTPEQWKSHRSYSCQVTHEGSTVEKTVAPTECS | |
| | GQPKAAPSVTLFPPSSEELQANKATLVCLISDFYPGAVTVAWKADSSPVKAGVE | 100 |
| | TTTPSKQSNNKYAASSYLSLTPEQWKSHKSYSCQVTHEGSTVEKTVAPTECS | |
| CH1 | ASTKGPSVFPLAPSSKSTSGGTAALGCLVKDYFPEPVTVSWNSGALTSGVHTFP | 19 |
| | AVLQSSGLYSLSSVVTVPSSSLGTQTYICNVNHKPSNTKVDKKV | |
| | ASTKGPSVFPLAPCSRSTSESTAALGCLVKDYFPEPVTVSWNSGALTSGVHTFPA | 101 |
| | VLQSSGLYSLSSVVTVPSSNFGTQTYTCNVDHKPSNTKVDKTV | |
| | ASTKGPSVFPLAPCSRSTSGGTAALGCLVKDYFPEPVTVSWNSGALTSGVHTFP | 102 |
| | AVLQSSGLYSLSSVVTVPSSSLGTQTYTCNVNHKPSNTKVDKRV | |
| | ASTKGPSVFPLAPCSRSTSESTAALGCLVKDYFPEPVTVSWNSGALTSGVHTFPA | 103 |
| | VLQSSGLYSLSSVVTVPSSSLGTKTYTCNVDHKPSNTKVDKRV | |

6. Fc sequences:

| | | | SEQ ID |
|---|---|---|---|
| Domain | No. | Amino acid sequence | NO: |
| Fc1 | G2D1 | SVFLFPPKPKDTLMISRTPEVTCVVVDVSHEAPEVQFNWYVDGVEV | 20 |
| or | | HNAKTKPREEQFNSTFRVVSVLTVVHQDWLNGKEYKCKVSNKGLP | |
| Fc2 | | APIEKTISKTKGQPREPQVYTLPPCRDELTKNQVSLWCLVKGFYPSDI | |
| | | AVEWESNGQPENNYKTTPPVLDSDGSFFLYSKLTVDKSRWQQGNVF | |
| | | SCSVMHEALHNHYTQKSLSLSPGK | |
| | G2D2 | SVFLFPPKPKDTLMISRTPEVTCVVVDVSHEAPEVQFNWYVDGVEV | 21 |
| | | HNAKTKPREEQFNSTFRVVSVLTVVHQDWLNGKEYKCKVSNKGLP | |
| | | APIEKTISKTKGQPREPQVCTLPPSRDELTKNQVSLSCAVKGFYPSDIA | |
| | | VEWESNGQPENNYKTTPPVLDSDGSFFLVSKLTVDKSRWQQGNVFS | |
| | | CSVMHEALHNHYTQKSLSLSPGK | |
| | M802P1 | SVFLFPPKPKDTLMISRTPEVTCVVVDVSHEDPEVKFNWYVDGVEV | 22 |
| | | HNAKTKPREEQYNSTYRVVSVLTVLHQDWLNGKEYKCKVSNKALP | |
| | | APIEKTISKAKGQPREPQVYTLPPSRDELTKNQVSLWCLVKGFYPSDI | |
| | | AVEWESNGQPENNYDTTPPVLDSDGSFFLYSDLTVDKSRWQQGNVF | |
| | | SCSVMHEALHNHYTQKSLSLSPGK | |
| | M802P2 | SVFLFPPKPKDTLMISRTPEVTCVVVDVSHEDPEVKFNWYVDGVEV | 23 |
| | | HNAKTKPREEQYNSTYRVVSVLTVLHQDWLNGKEYKCKVSNKALP | |
| | | APIEKTISKAKGQPREPQVYTLPPSRDELTKNQVSLTCRVKGFYPSDI | |
| | | AVEWESNGQPENNYKTTPPVLKSDGSFFLAAKLTVDKSRWQQGNV | |
| | | FSCSVMHEALHNHYTQKSLSLSPGK | |
| | NA | SVFLFPPKPKDTLMISRTPEVTCVVVDVSHEDPEVKFNWYVDGVEV | 24 |
| | | HNAKTKPREEQYNSTYRVVSVLTVLHQDWLNGKEYKCKVSNKALP | |
| | | APIEKTISKAKGQPREPQVYTLPPSRDELTKNQVSLTCLVKGFYPSDIA | |
| | | VEWESNGQPENNYKTTPPVLDSDGSFFLYSKLTVDKSRWQQGNVFS | |
| | | CSVMHEALHNHYTQKSLSLSPGK | |
| | XEFE | SVFLFPPKPKDTLMISRTPEVTCVVVFVSHEDPEVKFNWYVDGVEV | 25 |
| | | HNAKTKPREEQYESTYRVVSVLTVLHQDWLNGKEYKCKVSNKALP | |
| | | APEEKTISKAKGQPREPQVYTLPPSRDELTKNQVSLTCLVKGFYPSDI | |
| | | AVEWESNGQPENNYKTTPPVLDSDGSFFLYSKLTVDKSRWQQGNVF | |
| | | SCSVMHEALHNHYTQKSLSLSPGK | |
| | XDYLE | SVFLFPPKPKDTLMISRTPEVTCVVVYVSHEDPEVKFNWYVDGVEV | 26 |
| | | HNAKTKPREEQYDSLYRVVSVLTVLHQDWLNGKEYKCKVSNKALP | |
| | | APEEKTISKAKGQPREPQVYTLPPSRDELTKNQVSLTCLVKGFYPSDI | |
| | | AVEWESNGQPENNYKTTPPVLDSDGSFFLYSKLTVDKSRWQQGNVF | |
| | | SCSVMHEALHNHYTQKSLSLSPGK | |

7. Hinge region sequences:

| Domain | No. | Amino acid sequence | SEQ ID NO: |
|---|---|---|---|
| Hinge | Hin1 | EPKSCDKTHTCPPCPAPPVAGP | 27 |
| | Hin2 | DKTHTCPPCPAPPVAGP | 28 |
| | Hin3 | EPKSCDKTHT | 29 |

8. Linker peptide sequences:

| Domain | No. | Amino acid sequence | SEQ ID NO: |
|---|---|---|---|
| Linker | Lin1 | GRGRGS | 30 |
| peptide | Lin2 | APAPAPAPAPAPAPAP | 31 |
| | Lin3 | GGGGS | 32 |
| | Lin4 | GGGGSGGGGSGGGGS | 8 |

9. Monoclonal antibody sequences:

| | | | SEQ ID |
|---|---|---|---|
| Antibody | | Amino acid sequence | NO: |
| Anti-CLDN18.2 | Light | DIVMTQSPDSLAVSLGERATINCKSSQSLLNSGNQKNYLTWYQQ | 33 |
| monoclonal | chain | KPGQPPKLLIYWASTRDSGVPDRFSGSGSGTDFTLTISSLQAEDV | |
| antibody 2C6.9 | | AVYYCQNDFIFPLTFGGGTKVEIKRTVAAPSVFIFPPSDEQLKSGT | |
| | | ASVVCLLNNFYPREAKVQWKVDNALQSGNSQESVTEQDSKDS | |
| | | TYSLSSTLTLSKADYEKHKVYACEVTHQGLSSPVTKSFNRGEC | |
| | Heavy | QVQLQESGPGLVKPSETLSLTCTVSGFSLTRYGVSWIRQPPGKGL | 34 |
| | chain | EWIGVIWGEGNTNYNPSLKSRVTISKDSSKSQVSLKLSSVTAADT | |
| | | AVYYCARVNFGNALDYWGQGTLVTVSSASTKGPSVFPLAPSSK | |
| | | STSGGTAALGCLVKDYFPEPVTVSWNSGALTSGVHTFPAVLQSS | |
| | | GLYSLSSVVTVPSSSLGTQTYICNVNHKPSNTKVDKKVEPKSCD | |
| | | KTHTCPPCPAPELLGGPSVFLFPPKPKDTLMISRTPEVTCVVVDV | |
| | | SHEDPEVKFNWYVDGVEVHNAKTKPREEQYNSTYRVVSVLTV | |
| | | LHQDWLNGKEYKCKVSNKALPAPIEKTISKAKGQPREPQVYTL | |
| | | PPSRDELTKNQVSLTCLVKGFYPSDIAVEWESNGQPENNYKTTPP | |
| | | VLDSDGSFFLYSKLTVDKSRWQQGNVFSCSVMHEALHNHYTQK | |
| | | SLSLSPGK | |
| Anti-CLDN18.2 | Light | DIVMTQSPDSLAVSLGERATINCKSSQSLLNSGNQKNYLTWYQQ | 104 |
| monoclonal | chain | KPGQPPKLLIYWSSTRESGVPDRFSGSGSGTDFTLTISSLQAEDFA | |
| antibody 1E9.2 | | VYYCQNDYYYPLTFGGGTKVEIKRTVAAPSVFIFPPSDEQLKSGT | |
| | | ASVVCLLNNFYPREAKVQWKVDNALQSGNSQESVTEQDSKDS | |
| | | TYSLSSTLTLSKADYEKHKVYACEVTHQGLSSPVTKSFNRGEC | |
| | Heavy | EVQLVESGGGLVKPGGSLRLSCAASGFSFSNSAMSWVRQAPGK | 105 |
| | chain | GLEWVSTISSGDSYTYYADSVKGRFTISRDNAKNSLYLQMNSLR | |
| | | AEDTAVYYCARQGYGNALDYWGQGTLVTVSSASTKGPSVFPLA | |
| | | PSSKSTSGGTAALGCLVKDYFPEPVTVSWNSGALTSGVHTFPAV | |
| | | LQSSGLYSLSSVVTVPSSSLGTQTYICNVNHKPSNTKVDKKVEP | |
| | | KSCDKTHTCPPCPAPELLGGPSVFLFPPKPKDTLMISRTPEVTCV | |
| | | VVDVSHEDPEVKFNWYVDGVEVHNAKTKPREEQYNSTYRVVS | |
| | | VLTVLHQDWLNGKEYKCKVSNKALPAPIEKTISKAKGQPREPQ | |
| | | VYTLPPSRDELTKNQVSLTCLVKGFYPSDIAVEWESNGQPENNY | |
| | | KTTPPVLDSDGSFFLYSKLTVDKSRWQQGNVFSCSVMHEALHN | |
| | | HYTQKSLSLSPGK | |
| Anti-CLDN18.2 | Light | DIVMTQSPSSLTVTAGEKVTMSCKSSQSLLNSGNQKNYLTWYQ | 35 |
| monoclonal | chain | QKPGQPPKLLIYWASTRESGVPDRFTGSGSGTDFTLTISSVQAED | |
| antibody | | LAVYYCQNDYSYPFTFGSGTKLEIKRTVAAPSVFIFPPSDEQLKS | |
| IMAB362 | | GTASVVCLLNNFYPREAKVQWKVDNALQSGNSQESVTEQDSK | |
| | | DSTYSLSSTLTLSKADYEKHKVYACEVTHQGLSSPVTKSFNRGE | |
| | | C | |
| | Heavy | QVQLQQPGAELVRPGASVKLSCKASGYTFTSYWINWVKQRPG | 36 |
| | chain | QGLEWIGNIYPSDSYTNYNQKFKDKATLTVDKSSSTAYMQLSSP | |
| | | TSEDSAVYYCTRSWRGNSFDYWGQGTTLTVSSASTKGPSVFPL | |
| | | APSSKSTSGGTAALGCLVKDYFPEPVTVSWNSGALTSGVHTFPA | |
| | | VLQSSGLYSLSSVVTVPSSSLGTQTYICNVNHKPSNTKVDKKVE | |
| | | PKSCDKTHTCPPCPAPELLGGPSVFLFPPKPKDTLMISRTPEVTC | |
| | | VVVDVSHEDPEVKFNWYVDGVEVHNAKTKPREEQYNSTYRVV | |
| | | SVLTVLHQDWLNGKEYKCKVSNKALPAPIEKTISKAKGQPREPQ | |
| | | VYTLPPSRDELTKNQVSLTCLVKGFYPSDIAVEWESNGQPENNY | |
| | | KTTPPVLDSDGSFFLYSKLTVDKSRWQQGNVFSCSVMHEALHN | |
| | | HYTQKSLSLSPGK | |
| Anti-CD3 | Light | QTVVTQEPSLTVSPGGTVTLTCRSSTGAVTTSNYANWVQQKPGQ | 37 |
| monoclonal | chain | APRGLIGGTNKRAPGVPARFSGSLLGGKAALTLSGVQPEDEAEY | |
| antibody | | YCALWYSNLWVFGGGTKVEIKRTVAAPSVFIFPPSDEQLKSGTA | |
| | | SVVCLLNNFYPREAKVQWKVDNALQSGNSQESVTEQDSKDST | |
| | | YSLSSTLTLSKADYEKHKVYACEVTHQGLSSPVTKSFNRGEC | |
| | Heavy | QVQLVESGGGVVQPGRSLRLSCAASGFTFSTYAMNWVRQAPG | 38 |
| | chain | KGLEWVARIRSKYNNYATYYADSVKDRFTISRDDSKNTLYLQM | |
| | | NSLRAEDTAVYYCARHGNFGNSYVSWFAYWGQGTLVTVSSAST | |
| | | KGPSVFPLAPSSKSTSGGTAALGCLVKDYFPEPVTVSWNSGALT | |
| | | SGVHTFPAVLQSSGLYSLSSVVTVPSSSLGTQTYICNVNHKPSNT | |
| | | KVDKKVEPKSCDKTHTCPPCPAPPVAGPSVFLFPPKPKDTLMISR | |
| | | TPEVTCVVVDVSHEAPEVQFNWYVDGVEVHNAKTKPREEQFN | |
| | | STFRVVSVLTVVHQDWLNGKEYKCKVSNKGLPAPIEKTISKTKG | |
| | | QPREPQVYTLPPSRDELTKNQVSLTCLVKGFYPSDIAVEWESNG | |
| | | QPENNYKTTPPVLDSDGSFFLYSKLTVDKSRWQQGNVFSCSVM | |
| | | HEALHNHYTQKSLSLSPGK | |

10. Antigen amino acid sequences:

| Tumor antigen | | SEQ ID |
|---|---|---|
| (source) | Amino acid sequence | NO: |
| Human | MSTTTCQVVAFLLSILGLAGCIAATGMDMWSTQDLYDNPVTSVFQYEGL | 39 |
| CLDN18.1 | WRSCVRQSSGFTECRPYFTILGLPAMLQAVRALMIVGIVLGAIGLLVSIFAL | |
| (source: | KCIRIGSMEDSAKANMTLTSGIMFIVSGLCAIAGVSVFANMLVTNFWMST | |
| UniproKB- | ANMYTGMGGMVQTVQTRYTFGAALFVGWVAGGLTLIGGVMMCIACRG | |
| P56856-1) | LAPEETNYKAVSYHASGHSVAYKPGGFKASTGFGSNTKNKKIYDGGARTE | |
| | DEVQSYPSKHDYV | |
| Human | MAVTACQGLGFVVSLIGIAGIIAATCMDQWSTQDLYNNPVTAVFNYQGLW | 40 |
| CLDN18.2 | RSCVRESSGFTECRGYFTLLGLPAMLQAVRALMIVGIVLGAIGLLVSIFALK | |
| (source: | CIRIGSMEDSAKANMTLTSGIMFIVSGLCAIAGVSVFANMLVTNFWMSTA | |
| UniproKB- | NMYTGMGGMVQTVQTRYTFGAALFVGWVAGGLTLIGGVMMCIACRGL | |
| P56856-2) | APEETNYKAVSYHASGHSVAYKPGGFKASTGFGSNTKNKKIYDGGARTE | |
| | DEVQSYPSKHDYV | |
| Mouse | MATTTCQVVGLLLSLLGLAGCIAATGMDMWSTQDLYDNPVTAVFQYEGL | 41 |
| CLDN18A1.1 | WRSCVQQSSGFTECRPYFTILGLPAMLQAVRALMIVGIVLGVIGILVSIFAL | |
| (source: | KCIRIGSMDDSAKAKMTLTSGILFIISGICAIIGVSVFANMLVTNFWMSTAN | |
| UniproKB- | MYSGMGGMGGMVQTVQTRYTFGAALFVGWVAGGLTLIGGVMMCIACR | |
| P56857-1) | GLTPDDSNFKAVSYHASGQNVAYRPGGFKASTGFGSNTRNKKIYDGGART | |
| | EDDEQSHPTKYDYV | |
| Mouse | MATTTCQVVGLLLSLLGLAGCIAATGMDMWSTQDLYDNPVTAVFQYEGL | 42 |
| CLDN18A1.2 | WRSCVQQSSGFTECRPYFTILGLPAMLQAVRALMIVGIVLGVIGILVSIFAL | |
| (source: | KCIRIGSMDDSAKAKMTLTSGILFIISGICAIIGVSVFANMLVTNFWMSTAN | |
| UniproKB- | MYSGMGGMGGMVQTVQTRYTFGAALFVGWVAGGLTLIGGVMMCIACR | |
| P56857-2) | GLTPDDSK | |
| Mouse | MSVTACQGLGFVVSLIGFAGIIAATCMDQWSTQDLYNNPVTAVFNYQGL | 43 |
| CLDN18A2.1 | WRSCVRESSGFTECRGYFTLLGLPAMLQAVRALMIVGIVLGVIGILVSIFAL | |
| (source: | KCIRIGSMDDSAKAKMTLTSGILFIISGICAIIGVSVFANMLVTNFWMSTAN | |
| UniproKB- | MYSGMGGMGGMVQTVQTRYTFGAALFVGWVAGGLTLIGGVMMCIACR | |
| P56857-3) | GLTPDDSNFKAVSYHASGQNVAYRPGGFKASTGFGSNTRNKKIYDGGART | |
| | EDDEQSHPTKYDYV | |
| Mouse | MSVTACQGLGFVVSLIGFAGIIAATCMDQWSTQDLYNNPVTAVFNYQGL | 44 |
| CLDN18A2.2 | WRSCVRESSGFTECRGYFTLLGLPAMLQAVRALMIVGIVLGVIGILVSIFAL | |
| (source: | KCIRIGSMDDSAKAKMTLTSGILFIISGICAIIGVSVFANMLVTNFWMSTAN | |
| UniproKB- | MYSGMGGMGGMVQTVQTRYTFGAALFVGWVAGGLTLIGGVMMCIACR | |
| P56857-4) | GLTPDDSK | |
| Human CD3ϵ | DGNEEMGGITQTPYKVSISGTTVILTCPQYPGSEILWQHNDKNIGGDEDDK | 45 |
| (source: | NIGSDEDHLSLKEFSELEQSGYYVCYPRGSKPEDANFYLYLRARVCENCM | |
| UniProtKB- | EMD | |
| P07766) | | |
| Mouse CD3ϵ | MRWNTFWGILCLSLLAVGTCQDDAENIEYKVSISGTSVELTCPLDSDENL | 46 |
| (source: | KWEKNGQELPQKHDKHLVLQDFSEVEDSGYYVCYTPASNKNTYLYLKA | |
| UniProtKB- | RVCEYCVEVDLTAVAIIIIVDICITLGLLMVIYYWSKNRKAKAKPVTRGTG | |
| P22646) | AGSRPRGQNKE RPPPVPNPDYEPIRKGQRDLYSGLNQRAV | |

In some embodiments, the antigen-binding domain specifically binding to CLDN18.2 of the present invention (e.g., comprising a variable region set forth in SEQ ID NO: 5 and SEQ ID NO: 2 or CDRs therein, and/or comprising a variable region set forth in SEQ ID NO: 6 and SEQ ID NO: 3 or CDRs therein) has high binding activity to CLDN18.2 antigen and strong binding activity to both cells with high expression of CLDN18.2 and cells with medium/low expression of CLDN18.2. The inventors have found that a bispecific antibody (e.g., a bispecific antibody of the present invention in FIGS. 1-4) is obtained by assembling a specific antigen-binding domain specifically binding to CLDN18.2 with a specific antigen-binding domain specifically binding to CD3, and the bispecific antibody has one or more of the following advantages:

1. The method for preparing the bispecific antibody of the present invention is simple, and the prepared bispecific antibody has high expression level and high expression purity, and is easy to purify;
2. The bispecific antibody of the present invention has good stability, and particularly still maintains good stability in an acidic and thermal system;
3. The bispecific antibody of the present invention not only has strong binding effect to cells with high expression of CLDN18.2, but also has strong binding effect to cells with medium/low expression of CLDN18.2; as well as has strong killing activity to cells with high and medium/low expression of CLDN18.2;
4. The bispecific antibody of the present invention has no binding to other antigens, particularly CLDN18.1, and has no potential toxic and side effects, and the bispecific antibody of the present invention has no significant potential toxic and side effects while realizing the tumor inhibition effect;
5. The bispecific antibody of the present invention has high humanization degree, and has lower immunogenicity compared with other CD3-recruiting antibodies;
6. The bispecific antibody of the present invention has a CD3 sequence with moderate binding ability to T cell, which can activate T cell effectively while avoiding the potential immunotoxicity caused by over strong activation.
7. The bispecific antibody of the present invention having the structures in FIGS. 1-4 has higher stability and longer blood half-life, and are easier to prepare and purify as compared to BITE structures and other bispecific antibody structures.
8. The present invention makes intensive research and modification on a murine antibody, so that the murine antibody has extremely high humanization degree, retains (even is superior to) the functions and properties of the parent murine antibody, for example, the murine antibody binds to CLDN18.2 with high affinity and specificity, has lower immunogenic reaction and better safety.

### BRIEF DESCRIPTION OF THE DRAWINGS

FIG. 1 is an exemplary schematic diagram of the structure of a YBODY antibody (A) and the primary structure of each component protein (B).

FIG. 2 is an exemplary schematic diagram of the structure of a CSBODY antibody (A) and the primary structure of each component protein (B).

FIG. 3 is an exemplary schematic diagram of the structure of the bispecific antibody (A) and the primary structure of each component protein (B) of the present invention.

FIG. 4 is an exemplary schematic diagram of the structure of the bispecific antibody (A) and the primary structure of each component protein (B) of the present invention.

FIG. 5 shows the expression of CLDN18.2 antigen on the surface of cell strains 293 T-hCLDN18.2, NUGC4-hCLDN18.2, KATOIII-hCLDN18.2, CT26-hCLDN18.2, NUGC4, 293T-hCLDN18.1 and KATOIII.

FIG. 6 shows the binding effect of each antibody molecule to 293T-hCLDN18.1 cells.

FIG. 7 shows that the antibody-mediated hPBMC has in vitro killing effect on NUGC4 cells and NUGC4-hCLDN18.2 cells with an effector-to-target ratio of 10:1, and the hPBMC is detected after being incubated at 37° C. for 48 h; Y1, Y2, Y4, CS1 and CS2 bispecific antibodies have in vitro killing effect on NUGC4 cells (A) and NUGC4-hCLDN18.2 cells (B); Y6, Y7, Y8, CS4 and CS5 bispecific antibodies have in vitro killing effect on NUGC4 cells (C) and NUGC4-hCLDN18.2 cells (D); E+T is tumor cells mixed with effector cells without antibody added.

FIG. 8 shows that the antibody-mediated hPBMC has in vitro killing effect on 293T-hCLDN18.1 cells with an effector-to-target ratio of 10:1, and the hPBMC is detected after being incubated for 48 h; (A) Y1, Y2, Y4 and CS2 molecules have killing effect on 293T-hCLDN18.1 cells; (B) Y6, Y7, Y8, CS4 and CS5 molecules have killing effect on 293T-hCLDN18.1 cells; T+E is tumor cells mixed with effector cells without antibody added.

FIG. 9 shows that the antibody molecules induce the expression of immune cells CD69 and CD25 in the presence of NUGC4, NUGC4-hCLDN18.2 and 293T-hCLDN18.1 cells with an effector-to-target ratio of 10:1, and antibody molecules are detected after being incubated at 37° C. for 48 h; CS2, CS4, Y4 and Y6 molecules induce the expression of CD69 (A) and CD25 (D) on the surface of CD3+T cells in the presence of target cells NUGC4; CS2, CS4, Y4 and Y6 molecules induce the expression of CD69 (B) and CD25 (E) on the surface of CD3+T cells in the presence of target cells NUGC4-hCLDN18.2; CS4, Y4 and Y6 molecules induce the expression of CD69 (C) and CD25 (F) on the surface of CD3+T cells in the presence of target cells 293T-hCLDN18.1; wherein E+CS2, E+CS4, E+Y4, E+Y6 and E+isotype control groups indicate that the experimental group only has effector cells and CS2, CS4, Y4, Y6 or isotype control bispecific antibody; E+T is tumor cells mixed with effector cells without antibody added, and the effector cells are hPBMC cells.

FIG. 10 shows that the antibody molecule CS2 has proliferation promoting effect on immune cells in the presence of NUGC4, NUGC4-hCLDN18.2 and KATOIII cells; the CS2 molecule has the proliferation promoting effect on CD3+T cells in the presence of target cells NUGC4 (A), NUGC4-hCLDN182 (B) and non-target cells KATOIII (C); E is effector cell, i.e., hPBMC; Isotype is an isotype control bispecific antibody (anti-luciferase and anti-CD3); wherein E+CS2 indicates that the experimental group only has effector cells and CS2 bispecific antibody, and E+isotype control indicates that the experimental group only has effector cells and isotype control bispecific antibody; E+T is tumor cells mixed with effector cells without antibody added.

FIG. 11 shows the in vivo efficacy of the bispecific antibody CS4 in a CD3+T cell immune reconstituted B-NDG mouse subcutaneous NCI-N87-hCLDN18.2 xenograft tumor model, and the body weights (A) and tumor volumes (B) of mice in groups after the mice are randomly grouped for administration.

FIG. 12 shows the in vivo efficacy of the bispecific antibodies CS4 and Y6 in an hCD3E mouse CT26-hCLDN18.2 subcutaneous tumor model, and changes in body weights (A) and tumor volumes (B) of mice in groups after the mice are randomly grouped for administration.

FIG. 13 is an HPLC-SEC pattern of bispecific antibody CS2 molecule without acid treatment (A) and with treatment at pH 3.5 for 30 min (B), and an HPLC-SEC pattern of bispecific antibody CS4 molecule without acid treatment (C) and with treatment at pH 3.5 for 30 min (D).

### DETAILED DESCRIPTION

The embodiments of the present invention will be described in detail below with reference to the examples. Those skilled in the art will understand that the following examples are only used to illustrate the present invention, and should not be regarded as limiting the scope of the present invention. The cases without the specific descriptions of techniques or conditions were carried out according to the technologies or conditions described in the literature in the art (e.g., see, Guide to Molecular Cloning Experiments, authored by J. Sambrook et al., and translated by Huang Peitang et al., third edition, Science Press) or according to the product manual. Reagents or instruments used are commercially available conventional products if the manufacturers thereof are not specified.

### Example 1: Construction of Expression Vectors of Bispecific Antibodies

### 1. Construction of Expression Vector of Bispecific Antibody

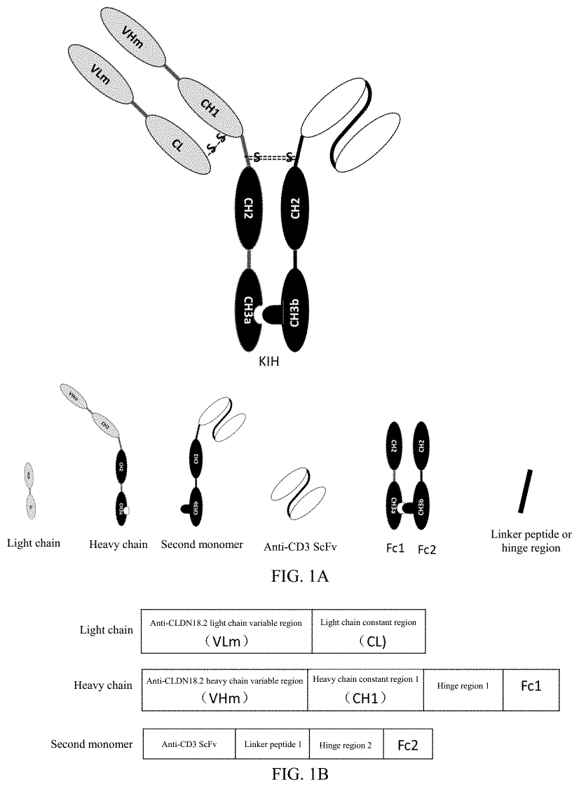

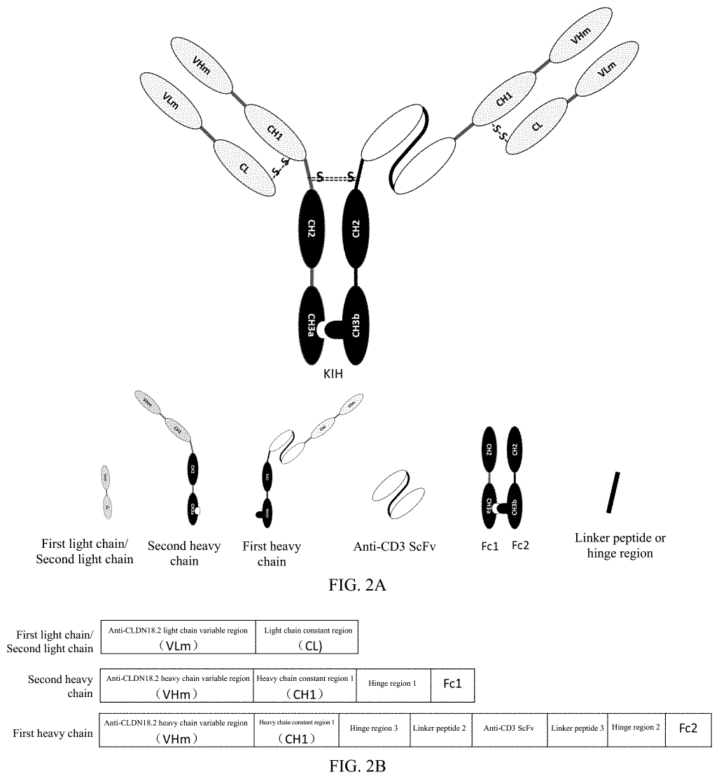

The DNA sequences of coding genes of chains corresponding to the bispecific antibodies were cloned into pcDNA3.1 eukaryotic expression vectors by the molecular biological method commonly used by those skilled in the art, wherein the bispecific antibodies comprise bispecific antibody molecules Y1, Y2, Y4, Y6, Y7, Y8, Y17, Y18 and SY1 having a YBODY structure (FIG. 1), and bispecific antibody molecules CS1, CS2, CS3, CS4, CS5, CS6, CS7, CS8, CS9, CS10, CS11 and SCS1 having a CSBODY structure (FIG. 2). The anti-CD3 scFv in bispecific antibody molecules Y1, Y2, Y4, Y6, Y7, Y8, Y17, Y18, SY1, CS1, CS2, CS3, CS4, CS5, CS6, CS7, CS8, CS9, CS10, CS11 and SCS1 has a structure from N—C terminus of VH-linker peptide 4-VL. In the bispecific antibody molecules CS1, CS2, CS3, CS4, CS5, CS6, CS7, CS8, CS9, CS10, CS11 and SCS1, the antigen-binding domain specifically binding to CLDN18.2 in a first heavy chain and a first light chain has VH and VL sequences identical to VH and VL sequences of the antigen-binding domain specifically binding to CLDN18.2 in a second heavy chain and a second light chain. Specific sequences of the bispecific antibodies are shown in Table 1 below.

TABLE 1
Sequences corresponding to bispecific antibodies

| Bi- | | | | |
|---|---|---|---|---|
| specific | | | | SEQ |
| anti- | | | | ID |
| bodies | Polypeptide | Domain | No. | NO: |
| Y1 | Light chain | VLm | 175D10 | 1 |
| | | CL | CL | 18 |
| | Heavy chain | VHm | 175D10 | 4 |
| | | CH1 | CH1 | 19 |
| | | Hinge region 1 | Hin1 | 27 |
| | | Fc1 | G2D1 | 20 |
| | Second monomer | anti-CD3 VH | 2a5ga | 7 |
| | | Linker peptide 4 | Lin4 | 8 |
| | | Anti-CD3 VL | 2a5ga | 9 |
| | | Linker peptide 1 | Lin1 | 30 |
| | | Hinge region 2 | Hin2 | 28 |
| | | Fc2 | G2D2 | 21 |
| Y2 | Light chain | VLm | 175D10 | 1 |
| | | CL | CL | 18 |
| | Heavy chain | VHm | 175D10 | 4 |
| | | CH1 | CH1 | 19 |
| | | Hinge region 1 | Hin1 | 27 |
| | | Fc1 | G2D1 | 20 |
| | Second monomer | anti-CD3 VH | 2a5 | 10 |
| | | Linker peptide 4 | Lin4 | 8 |
| | | Anti-CD3 VL | 2a5 | 11 |
| | | Linker peptide 1 | Lin1 | 30 |
| | | Hinge region 2 | Hin2 | 28 |
| | | Fc2 | G2D2 | 21 |
| Y4 | Light chain | VLm | 1E9.2 | 2 |
| | | CL | CL | 18 |
| | Heavy chain | VHm | 1E9.2 | 5 |
| | | CH1 | CH1 | 19 |
| | | Hinge region 1 | Hin1 | 27 |
| | | Fc1 | G2D1 | 20 |
| | Second monomer | anti-CD3 VH | 2a5 | 10 |
| | | Linker peptide 4 | Lin4 | 8 |
| | | Anti-CD3 VL | 2a5 | 11 |
| | | Linker peptide 1 | Lin1 | 30 |
| | | Hinge region 2 | Hin2 | 28 |
| | | Fc2 | G2D2 | 21 |
| Y6 | Light chain | VLm | 2C6.9 | 3 |
| | | CL | CL | 18 |
| | Heavy chain | VHm | 2C6.9 | 6 |
| | | CH1 | CH1 | 19 |
| | | Hinge region 1 | Hin1 | 27 |
| | | Fc1 | G2D1 | 20 |
| | Second monomer | anti-CD3 VH | 2a5 | 10 |
| | | Linker peptide 4 | Lin4 | 8 |
| | | Anti-CD3 VL | 2a5 | 11 |
| | | Linker peptide 1 | Lin1 | 30 |
| | | Hinge region 2 | Hin2 | 28 |
| | | Fc2 | G2D2 | 21 |
| Y7 | Light chain | VLm | 2C6.9 | 3 |
| | | CL | CL | 18 |
| | Heavy chain | VHm | 2C6.9 | 6 |
| | | CH1 | CH1 | 19 |
| | | Hinge region 1 | Hin1 | 27 |
| | | Fc1 | G2D1 | 20 |
| | Second monomer | anti-CD3 VH | Di6-2 | 12 |
| | | Linker peptide 4 | Lin4 | 8 |
| | | Anti-CD3 VL | Di6-2 | 13 |
| | | Linker peptide 1 | Lin1 | 30 |
| | | Hinge region 2 | Hin2 | 28 |
| | | Fc2 | G2D2 | 21 |
| Y8 | Light chain | VLm | 2C6.9 | 3 |
| | | CL | CL | 18 |
| | Heavy chain | VHm | 2C6.9 | 6 |
| | | CH1 | CH1 | 19 |
| | | Hinge region 1 | Hin1 | 27 |
| | | Fc1 | G2D1 | 20 |
| | Second monomer | anti-CD3 VH | L2K | 14 |
| | | Linker peptide 4 | Lin4 | 8 |
| | | Anti-CD3 VL | L2K | 15 |
| | | Linker peptide 1 | Lin1 | 30 |
| | | Hinge region 2 | Hin2 | 28 |
| | | Fc2 | G2D2 | 21 |
| Y17 | Light chain | VLm | 1E9.2 | 2 |
| | | CL | CL | 18 |
| | Heavy chain | VHm | 1E9.2 | 5 |
| | | CH1 | CH1 | 19 |
| | | Hinge region 1 | Hin1 | 27 |
| | | Fc1 | G2D1 | 20 |
| | Second monomer | anti-CD3 VH | Di6-2 | 12 |
| | | Linker peptide 4 | Lin4 | 8 |
| | | Anti-CD3 VL | Di6-2 | 13 |
| | | Linker peptide 1 | Lin1 | 30 |
| | | Hinge region 2 | Hin2 | 28 |
| | | Fc2 | G2D2 | 21 |
| Y18 | Light chain | VLm | 1E9.2 | 2 |
| | | CL | CL | 18 |
| | Heavy chain | VHm | 1E9.2 | 5 |
| | | CH1 | CH1 | 19 |
| | | Hinge region 1 | Hin1 | 27 |
| | | Fc1 | G2D1 | 20 |
| | Second monomer | anti-CD3 VH | L2K | 14 |
| | | Linker peptide 4 | Lin4 | 8 |
| | | Anti-CD3 VL | L2K | 15 |
| | | Linker peptide 1 | Lin1 | 30 |
| | | Hinge region 2 | Hin2 | 28 |
| | | Fc2 | G2D2 | 21 |
| SY1 | Light chain | VLm | 2C6.9 | 3 |
| | | CL | CL | 18 |
| | Heavy chain | VHm | 2C6.9 | 6 |
| | | CH1 | CH1 | 19 |
| | | Hinge region 1 | Hin1 | 27 |
| | | Fc1 | G2D1 | 20 |
| | Single chain | anti-CD3 VH | 2C11 | 16 |
| | | Linker peptide 4 | Lin4 | 8 |
| | | Anti-CD3 VL | 2C11 | 17 |
| | | Linker peptide 1 | Lin1 | 30 |
| | | Hinge region 2 | Hin2 | 28 |
| | | Fc2 | G2D2 | 21 |
| CS1 | First light chain/ | VLm | 175D10 | 1 |
| | Second light chain | CL | CL | 18 |
| | Second heavy | VHm | 175D10 | 4 |
| | chain | CH1 | CH1 | 19 |
| | | Hinge region 1 | Hin1 | 27 |
| | | Fc1 | G2D1 | 20 |
| | | VHm | 175D10 | 4 |
| | | CH1 | CH1 | 19 |
| | | Hinge region 3 | Hin3 | 29 |
| | | Linker peptide 2 | Lin2 | 31 |
| | First heavy chain | anti-CD3 VH | 2a5ga | 7 |
| | | Linker peptide 4 | Lin4 | 8 |
| | | Anti-CD3 VL | 2a5ga | 9 |
| | | Linker peptide 3 | Lin3 | 32 |
| | | Hinge region 2 | Hin2 | 28 |
| | | Fc2 | G2D2 | 21 |
| CS2 | First light chain/ | VLm | 175D10 | 1 |
| | Second light chain | CL | CL | 18 |
| | | VHm | 175D10 | 4 |
| | Second heavy | CH1 | CH1 | 19 |
| | chain | Hinge region 1 | Hin1 | 27 |
| | | Fc1 | G2D1 | 20 |
| | | VHm | 175D10 | 4 |
| | | CH1 | CH1 | 19 |
| | | Hinge region 3 | Hin3 | 29 |
| | | Linker peptide 2 | Lin2 | 31 |
| | First heavy chain | anti-CD3 VH | 2a5 | 10 |
| | | Linker peptide 4 | Lin4 | 8 |
| | | Anti-CD3 VL | 2a5 | 11 |
| | | Linker peptide 3 | Lin3 | 32 |
| | | Hinge region 2 | Hin2 | 28 |
| | | Fc2 | G2D2 | 21 |
| CS3 | First light chain/ | VLm | 1E9.2 | 2 |
| | Second light chain | CL | CL | 18 |
| | | VHm | 1E9.2 | 5 |
| | Second heavy | CH1 | CH1 | 19 |
| | chain | Hinge region 1 | Hin1 | 27 |
| | | Fc1 | G2D1 | 20 |
| | | VHm | 1E9.2 | 5 |
| | | CH1 | CH1 | 19 |
| | | Hinge region 3 | Hin3 | 29 |
| | | Linker peptide 2 | Lin2 | 31 |
| | First heavy chain | anti-CD3 VH | 2a5 | 10 |
| | | Linker peptide 4 | Lin4 | 8 |
| | | Anti-CD3 VL | 2a5 | 11 |
| | | Linker peptide 3 | Lin3 | 32 |
| | | Hinge region 2 | Hin2 | 28 |
| | | Fc2 | G2D2 | 21 |
| CS4 | First light chain/ | VLm | 2C6.9 | 3 |
| | Second light chain | CL | CL | 18 |
| | | VHm | 2C6.9 | 6 |
| | Second heavy | CH1 | CH1 | 19 |
| | chain | Hinge region 1 | Hin1 | 27 |
| | | Fc1 | G2D1 | 20 |
| | | VHm | 2C6.9 | 6 |
| | | CH1 | CH1 | 19 |
| | | Hinge region 3 | Hin3 | 29 |
| | First heavy chain | Linker peptide 2 | Lin2 | 31 |
| | | anti-CD3 VH | 2a5 | 10 |
| | | Linker peptide 4 | Lin4 | 8 |
| | | Anti-CD3 VL | 2a5 | 11 |
| | | Linker peptide 3 | Lin3 | 32 |
| | | Hinge region 2 | Hin2 | 28 |
| | | Fc2 | G2D2 | 21 |
| CS5 | First light chain/ | VLm | 2C6.9 | 3 |
| | Second light chain | CL | CL | 18 |
| | | VHm | 2C6.9 | 6 |
| | Second heavy | CH1 | CH1 | 19 |
| | chain | Hinge region 1 | Hin1 | 27 |
| | | Fc1 | G2D1 | 20 |
| | | VHm | 2C6.9 | 6 |
| | | CH1 | CH1 | 19 |
| | | Hinge region 3 | Hin3 | 29 |
| | | Linker peptide 2 | Lin2 | 31 |
| | First heavy chain | anti-CD3 VH | 2a5ga | 7 |
| | | Linker peptide 4 | Lin4 | 8 |
| | | Anti-CD3 VL | 2a5ga | 9 |
| | | Linker peptide 3 | Lin3 | 32 |
| | | Hinge region 2 | Hin2 | 28 |
| | | Fc2 | G2D2 | 21 |
| CS6 | First light chain/ | VLm | 175D10 | 1 |
| | Second light chain | CL | CL | 18 |
| | | VHm | 175D10 | 4 |
| | Second heavy | CH1 | CH1 | 19 |
| | chain | Hinge region 1 | Hin1 | 27 |
| | | Fc1 | G2D1 | 20 |
| | | VHm | 175D10 | 4 |
| | | CH1 | CH1 | 19 |
| | | Hinge region 3 | Hin3 | 29 |
| | | Linker peptide 2 | Lin2 | 31 |
| | First heavy chain | anti-CD3 VH | Di6-2 | 12 |
| | | Linker peptide 4 | Lin4 | 8 |
| | | Anti-CD3 VL | Di6-2 | 13 |
| | | Linker peptide 3 | Lin3 | 32 |
| | | Hinge region 2 | Hin2 | 28 |
| | | Fc2 | G2D2 | 21 |
| CS7 | First light chain/ | VLm | 1E9.2 | 2 |
| | Second light chain | CL | CL | 18 |
| | | VHm | 1E9.2 | 5 |
| | Second heavy | CH1 | CH1 | 19 |
| | chain | Hinge region 1 | Hin1 | 27 |
| | | Fc1 | G2D1 | 20 |
| | | VHm | 1E9.2 | 5 |
| | | CH1 | CH1 | 19 |
| | | Hinge region 3 | Hin3 | 29 |
| | | Linker peptide 2 | Lin2 | 31 |
| | First heavy chain | anti-CD3 VH | Di6-2 | 12 |
| | | Linker peptide 4 | Lin4 | 8 |
| | | Anti-CD3 VL | Di6-2 | 13 |
| | | Linker peptide 3 | Lin3 | 32 |
| | | Hinge region 2 | Hin2 | 28 |
| | | Fc2 | G2D2 | 21 |
| CS8 | First light chain/ | VLm | 2C6.9 | 3 |
| | Second light chain | CL | CL | 18 |
| | Second heavy | VHm | 2C6.9 | 6 |
| | | CH1 | CH1 | 19 |
| | chain | Hinge region 1 | Hin1 | 27 |
| | | Fc1 | G2D1 | 20 |
| | First heavy chain | VHm | 2C6.9 | 6 |
| | | CH1 | CH1 | 19 |
| | | Hinge region 3 | Hin3 | 29 |
| | | Linker peptide 2 | Lin2 | 31 |
| | | anti-CD3 VH | Di6-2 | 12 |
| | | Linker peptide 4 | Lin4 | 8 |
| | | Anti-CD3 VL | Di6-2 | 13 |
| | | Linker peptide 3 | Lin3 | 32 |
| | | Hinge region 2 | Hin2 | 28 |
| | | Fc2 | G2D2 | 21 |
| CS9 | First light chain/ | VLm | 175D10 | 1 |
| | Second light chain | CL | CL | 18 |
| | Second heavy | VHm | 175D10 | 4 |
| | chain | CH1 | CH1 | 19 |
| | | Hinge region 1 | Hin1 | 27 |
| | | Fc1 | G2D1 | 20 |
| | First heavy chain | VHm | 175D10 | 4 |
| | | CH1 | CH1 | 19 |
| | | Hinge region 3 | Hin3 | 29 |
| | | Linker peptide 2 | Lin2 | 31 |
| | | anti-CD3 VH | L2K | 14 |
| | | Linker peptide 4 | Lin4 | 8 |
| | | Anti-CD3 VL | L2K | 15 |
| | | Linker peptide 3 | Lin3 | 32 |
| | | Hinge region 2 | Hin2 | 28 |
| | | Fc2 | G2D2 | 21 |
| CS10 | First light chain/ | VLm | 1E9.2 | 2 |
| | Second light chain | CL | CL | 18 |
| | Second heavy | VHm | 1E9.2 | 5 |
| | chain | CH1 | CH1 | 19 |
| | | Hinge region 1 | Hin1 | 27 |
| | | Fc1 | G2D1 | 20 |
| | First heavy chain | VHm | 1E9.2 | 5 |
| | | CH1 | CH1 | 19 |
| | | Hinge region 3 | Hin3 | 29 |
| | | Linker peptide 2 | Lin2 | 31 |
| | | anti-CD3 VH | L2K | 14 |
| | | Linker peptide 4 | Lin4 | 8 |
| | | Anti-CD3 VL | L2K | 15 |
| | | Linker peptide 3 | Lin3 | 32 |
| | | Hinge region 2 | Hin2 | 28 |
| | | Fc2 | G2D2 | 21 |
| CS11 | First light chain/ | VLm | 2C6.9 | 3 |
| | Second light chain | CL | CL | 18 |
| | Second heavy | VHm | 2C6.9 | 6 |
| | chain | CH1 | CH1 | 19 |
| | | Hinge region 1 | Hin1 | 27 |
| | | Fc1 | G2D1 | 20 |
| | First heavy chain | VHm | 2C6.9 | 6 |
| | | CH1 | CH1 | 19 |
| | | Hinge region 3 | Hin3 | 29 |
| | | Linker peptide 2 | Lin2 | 31 |
| | | anti-CD3 VH | L2K | 14 |
| | | Linker peptide 4 | Lin4 | 8 |
| | | Anti-CD3 VL | L2K | 15 |
| | | Linker peptide 3 | Lin3 | 32 |
| | | Hinge region 2 | Hin2 | 28 |
| | | Fc2 | G2D2 | 21 |
| SCS1 | First light chain/ | VLm | 2C6.9 | 3 |
| | Second light chain | CL | CL | 18 |
| | Second heavy | VHm | 2C6.9 | 6 |
| | chain | CH1 | CH1 | 19 |
| | | Hinge region 1 | Hin1 | 27 |
| | | Fc1 | G2D1 | 20 |
| | First heavy chain | VHm | 2C6.9 | 6 |
| | | CH1 | CH1 | 19 |
| | | Hinge region 3 | Hin3 | 29 |
| | | Linker peptide 2 | Lin2 | 31 |
| | | anti-CD3 VH | 2C11 | 16 |
| | | Linker peptide 4 | Lin4 | 8 |
| | | Anti-CD3 VL | 2C11 | 17 |
| | | Linker peptide 3 | Lin3 | 32 |
| | | Hinge region 2 | Hin2 | 28 |
| | | Fc2 | G2D2 | 21 |

Note:
the variable region sequences of Y1 and Y2 are from US8425902B2 and PCT/CN2019/075901. The antigen-binding domains specifically binding to CLDN18.2 of Y1, Y2, CS1, CS2, CS6 and CS9 are from 175D10; the antigen-binding domains specifically binding to CLDN18.2 of Y4, Y17, Y18, CS3, CS7 and CS10 are from 1E9.2; the antigen-binding domains specifically binding to CLDN18.2 of Y6, Y7, Y8, SY1, CS4, CS5, CS8, CS11 and SCS1 are from 2C6.9.

### Example 2: Expression and Purification of Bispecific Antibodies

The plasmid was extracted according to a conventional plasmid extraction method and used to transfect 293 cells or CHO-S cells, and the transfection reagent may be Lipofectamine2000 (Thermo fisher, Catalog No. 11668019). The transfected cells were cultured in 293 cells or CHO cells in suspension shaking at 37° C. in a 5% CO₂ shaker for 7-10 days. The supernatant was collected by centrifugation at 3000×g and filtered through a 0.22 μm filter, and then purified by protein A affinity chromatography and cation exchange chromatography to obtain the bispecific antibodies. The concentration of the purified bispecific antibody was determined by UV absorbance at 280 nm and the corresponding extinction coefficient for each protein. The purified protein was analyzed by SDS-PAGE, and the molecular weight of the bispecific antibody with a structure of YBODY was about 125 KD, and the molecular weight of the bispecific antibody with a structure of CSBODY was about 175 KD. The high-polymer content of each bispecific antibody was tested by high performance size exclusion chromatography (HPLC-SEC). The content of the bispecific antibody in the collected supernatant was 10.5-240 mg/L, and the purity of the bispecific antibody finally obtained through purification was more than 95% through HPLC-SEC detection.

### Example 3: Detection of Affinity of Bispecific Antibody CLDN18.2 Terminus

The affinity of the bispecific antibody CLDN18.2 terminus included in the present invention utilized flow cytometry to detect the binding of the bispecific antibody to the target antigen CLDN18.2 (SEQ ID NO: 40) of the corresponding cell. The present invention adopted human gastric cancer cell strain NUGC4 and human CLDN18.2 over-expressed cell strains including human embryonic kidney cell strains 293T-hCLDN18.2, human gastric cancer cell strains NUGC4-hCLDN18.2, human gastric cancer cell strains KATOIII-hCLDN18.2 and mouse colon cancer cell strains CT26-hCLDN18.2 as CLDN18.2 antigen expression-positive cells, and adopted human gastric cancer cell strains KATOIII and human CLDN18.1 (SEQ ID NO: 39) over-expressed human embryonic kidney cell strains 293T-hCLDN18.1 as CLDN18.2 negative cells. The CT26-hCLDN18.2, 293T-hCLDN18.1 and 293T-hCLDN18.2 cells were purchased from KYinno Biotechnology (Beijing) Co., Ltd, the NUGC4 cells were purchased from Nanjing Kebai Biotechnology Co., Ltd., and the KATOIII cells were purchased from Chinese Academy of Sciences, Shanghai Institutes for Biological Sciences. KATOIII-hCLDN18.2 and NUGC4-hCLDN18.2 cell strains were obtained by transfecting KATOIII and NUGC4 cells with lentivirus containing the gene encoding human CLDN18.2 and antibiotic pressure screening according to the relevant reagent instructions, for example, see the method as described in the instruction of Lenti-Pac HIV lentivirus package kit (GeneCopoeia, HPK-LvTR-20).

### 1. Detection of Expression of CLDN18.2 on the Surface of Cells

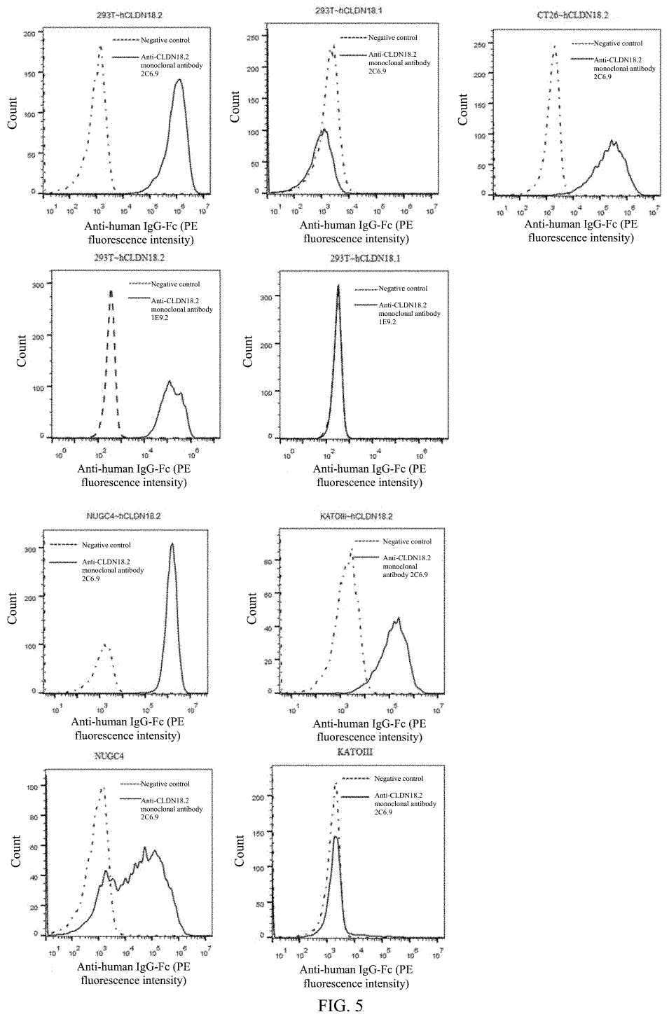

Cells were collected and resuspended in buffer (PBS+1% FBS) at 1×10⁴ cells/well in a 96-well plate at 100 μL per well. Then the mixture was centrifuged at 350×g for 5 min, and the supernatant was removed. Anti-CLDN18.2 monoclonal antibody 2C6.9 (with a light chain sequence set forth in SEQ ID NO: 33 and a heavy chain sequence set forth in SEQ ID NO: 34, prepared by Wuhan YZY Biopharma Co. Ltd.) and anti-CLDN18.2 monoclonal antibody 1E9.2 (with a light chain sequence set forth in SEQ ID NO: 104 and a heavy chain sequence set forth in SEQ ID NO: 105, prepared by Wuhan YZY Biopharma Co. Ltd.) were diluted to 1000 nM with buffer, added to a 96-well plate, resuspended, incubated at 4° C. in the dark for 1 h and centrifuged to remove supernatant; the plate was washed twice with buffer, resuspended in diluted PE-labeled anti-human IgG Fc antibody (Biolegend, 409304), incubated at 4° C. in the dark for 30 min, then washed twice with buffer, resuspended in 100 μL buffer, and detected on a flow cytometer (BD Accuri™ C6). As shown in FIG. 5, both anti-CLDN18.2 monoclonal antibody 2C6.9 and monoclonal antibody 1E9.2 could effectively bind to 293T-hCLDN18.2 but not to 293T-hCLDN18.1 cells, which indicated specificity of binding of anti-CLDN18.2 monoclonal antibodies 2C6.9 and 1E9.2 to the CLDN18.2 antigen. Meanwhile, NUGC4-hCLDN18.2, KATOIII-hCLDN18.2 and CT26-hCLDN18.2 cells after detection had strong binding effect to anti-CLDN18.2 monoclonal antibody 2C6.9, which indicated that the cell strains highly expressed human CLDN18.2 antigen; the human gastric cancer cell strain NUGC4 had certain binding effect to the CLDN18.2 monoclonal antibody 2C6.9, which indicated that moderately expressed human CLDN18.2 antigen; however, the human gastric cancer cell strains KATOIII had no binding effect to the CLDN18.2 monoclonal antibody, which indicated that the human gastric cancer cell strains KATOIII did not express the human CLDN18.2 antigen.

### 2. Detection of Binding Activity of Bispecific Antibodies to CLDN18.2 Positive Cells Using Flow Cytometry Analysis

NUGC4 and NUGC4-hCLDN18.2 cells were cultured, digested with pancreatin and centrifuged to collect cells. The cells were collected and resuspended in buffer (PBS+1% FBS) at 1×10⁴ cells/well in a 96-well plate at 100 μL per well. Then the mixture was centrifuged at 350×g for 5 min, and the supernatant was removed. The bispecific antibody was diluted to 3000 nM with buffer and serially 3- or 4-fold diluted to the 11^{th} concentration, and then the bispecific antibody was added to a 96-well plate at 100 μL/well, resuspended, incubated at 4° C. in the dark for 1 h and centrifuged to remove supernatant; the plate was washed twice with buffer, resuspended in diluted PE-labeled anti-human IgG Fc antibody (Biolegend, 409304), incubated at 4° C. in the dark for 30 min, then washed twice with buffer, resuspended in 100 μL buffer, and detected on a flow cytometer (BD Accuri™ C6). Y4, Y6, Y7, Y8, CS3, CS4, CS5, SY1 and SCS1 all had significant binding effects to NUGC4 and NUGC4-hCLDN18.2 cells, and specific binding EC₅₀ values are shown in Table 2. In the same experimental system, other bispecific antibodies such as Y17, Y18, CS6, CS7, CS8, CS9, CS10 and CS11 of the present invention all had binding EC₅₀ values of less than 50 nM to NUGC4-hCLDN18.2 cells; Y17, Y18, CS7, CS8, CS10 and CS11 all had binding EC₅₀ values of less than 100 nM to NUGC4 cells; IMAB362 monoclonal antibody (specifically binding to CLDN18.2, with a light chain sequence set forth in SEQ ID NO: 35 and a heavy chain sequence set forth in SEQ ID NO: 36, with reference to U.S. Pat. No. 8,425,902B2, prepared by Wuhan YZY Biopharma Co. Ltd.) did not significantly bind to NUGC4 cells, and had a binding EC₅₀ value of 10.79 nM to NUGC4-hCLDN18.2 cells. Y4, Y6, Y7, Y8, Y17, Y18, CS3, CS4, CS5, CS7, CS8, CS10, CS11, SY1 and SCS1 had superior binding effect to NUGC4 cells than IMAB362.

TABLE 2
Binding ability of bispecific antibodies
to NUGC4 and NUGC4-hCLDN18.2 cells

| Bispecific | EC₅₀ (nM) | |
|---|---|---|
| antibodies | NUGC4 | NUGC4~hCLDN18.2 |
| Y1 | No significant binding effect | Not reach plateau |
| Y2 | No significant binding effect | Not reach plateau |
| Y4 | 58.94 | 24.36 |
| Y6 | 183.5 | 45.91 |
| Y7 | 127.4 | 25.42 |
| Y8 | 75.11 | 46.93 |
| CS1 | Not reach plateau | 14.63 |
| CS2 | Not reach plateau | 35.97 |
| CS3 | 63.95 | 31.48 |
| CS4 | 23.42 | 18.87 |
| CS5 | 25.72 | 18.18 |
| SY1 | 303.8 | 125.1 |
| SCSI | 29.12 | 25.37 |

### 3. Detection of Binding Activity of Bispecific Antibodies to CLDN18.2 Negative Cells Using Flow Cytometry Analysis

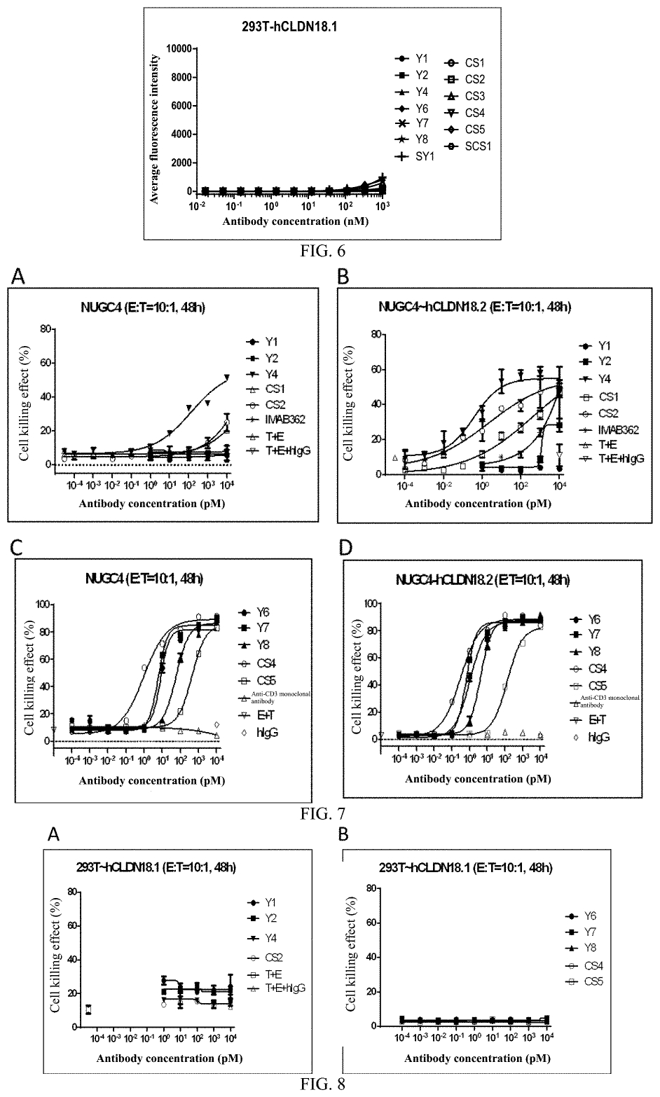

The step 3.2 was repeated for detection by using 293T-hCLDN18.1 cells. As shown in FIG. 6, bispecific antibodies had no significant binding effect to 293T-hCLDN18.1 cells, and had weak binding effect under the condition of high dose, and possibly non-specific binding effect. Under the same detection conditions, other bispecific antibodies such as Y17, Y18, CS6, CS7, CS8, CS9, CS10 and CS11 of the present invention had no significant binding effect with 293T-hCLDN18.1 cells.

### Example 4: Detection of Affinity of Bispecific Antibody CD3 Terminus by BIACORE

A human CD3 antigen (SEQ ID NO: 45) was fixed on a CM5 chip by adopting an amino coupling method, wherein the antigen coupling amount was 1500 RU, when the binding activity of the CD3 antigen terminus was detected, a sample was diluted to an initial concentration by adopting 1×HBS-EP+buffer, and then serially 2-fold diluted to the 4^{th} concentration, and then the samples were added to Biacore T200 from a low concentration to a high concentration, wherein the binding flow rate was 30 μL/min, the binding time was 120 s, and the dissociation time was 300 s; the chip was regenerated by adopting a pH1.5 Glycine solution, the regeneration flow rate was 10 μL/min, and the regeneration time was 30 s. After the detection was completed, data fitting was carried out on the result pattern by adopting Biacore T200 Evaluation Software in a 1:1 binding fitting mode to obtain an equilibrium dissociation constant (KD). As shown in Table 3, Y2, Y4, Y6, Y7, Y8, CS2, CS3 and CS4 all had strong binding effect to human CD3 antigen. In the same experiment system, other bispecific antibodies such as Y17, Y18, CS6, CS7, CS8, CS9, CS10 and CS11 of the present invention had stronger binding effect to human CD3 antigen with KD value less than 15 nM.

TABLE 3
Detection of binding ability of bispecific
antibodies to human CD3 antigen by BIACORE

| | Bispecific antibodies | | | | | | | | |
|---|---|---|---|---|---|---|---|---|---|
| | Y1 | Y2 | Y4 | Y6 | Y7 | Y8 | CS2 | CS3 | CS4 |
| KD | 1975 | 20.32 | 5.742 | 5.773 | 4.856 | 22.22 | 3.100 | 10.00 | 12.03 |
| (nM) | | | | | | | | | |

### Example 5: Detection of Affinity of Bispecific Antibody CD3 Terminus by BIACORE

The experiment in Example 4 was repeated using the murine CD3 antigen (SEQ ID NO: 46). The results are shown in Table 4, wherein SY1 and SCS1 had binding KD values of 79.30 nM and 94.10 nM to murine CD3 antigen, respectively.

TABLE 4
Detection of binding ability of
bispecific antibodies to murine CD3
antigen by BIACORE

| Bispecific antibodies | KD (nM) |
|---|---|
| SY1 | 79.30 |
| SCS1 | 94.10 |

### Example 6: Detection of In Vitro Killing Effect Mediated by Bispecific Antibodies

### 1. Isolation of Human Peripheral Blood Mononuclear Cells

The collected peripheral blood was transferred to a 50 mL centrifuge tube, and added with PBS in a volume ratio 1:1 for dilution, and then the mixture was gently mixed. A 50 mL centrifuge tube was added with 10-15 mL of Ficoll solution, then the diluted blood was gently added dropwise on the Ficoll upper layer of the centrifuge tube, and then centrifuged at 400×g for 30 min (the rising speed was set as 1, and the falling speed was set as 0). After the centrifugation was completed, the middle white peripheral blood mononuclear cell (PBMC) layer was sucked into another clean 50 mL centrifuge tube, the tube was added with 2-fold volume of PBS and centrifuged at 400×g for 10 min to remove the supernatant; the above operation was repeated, and the tube was added with red blood cell lysis buffer, gently pipetted and mixed well, and then lysed for 4-5 min at room temperature. The tube was added with PBS and washed 1-2 times. The cells were resuspended in culture medium according to the needs of the experiment and counted for subsequent experiments.

### 2. Detection of In Vitro Killing Effect of Bispecific Antibody on Target Cells by PI Single-Staining Method

Bispecific antibody mediated in vitro killing effect was detected by using the human PBMC (hPBMC) obtained by isolation as effector cells and CLDN18.2 expressing cells as target cells. The target cells were digested into single-cell suspensions with pancreatin, and centrifuged at 300×g for 5 min to collect cells; the cells were stained with 5 μM of 5,6-carboxyfluorescein diacetate, succinimidyl ester (CFSE) (37° C., 15 min), washed twice with complete medium and counted on a Vi-Cell cell counter (Beckman); then the cells were added to a 96-well plate at 2×10⁴ cells/100 μL/well according to the experimental design. The prepared 4×antibody molecule was added at 50 μL/well and the hPBMC was counted on a Cellometer cell counter and then placed in a 96-well plate (2×10⁵ cells/50 μL/well at an effector-to-target ratio of 10:1). The cell culture plate was placed in a cell incubator for culturing for 48 h, after the cells were digested into single cell suspension, propidium iodide (PI) solution with a final concentration of 1 μg/mL was added, after the cells were incubated for 10 min, the cells were detected on a flow cytometer (BD Accuri™ C6), and the percentage of CFSE+PI+double positive cells in CFSE+positive cells was analyzed. Anti-CD3 monoclonal antibody (with a light chain sequence set forth in SEQ ID NO: 37 and a heavy chain sequence set forth in SEQ ID NO: 38, prepared by Wuhan YZY Biopharma Co. Ltd.) was used as the CD3 monoclonal antibody control.

As shown in FIG. 7 and Table 5, bispecific antibodies Y4, Y6, Y7, Y8, CS3, CS4 and CS5 all had strong killing effects on NUGC4-hCLDN18.2 cells and NUGC4 cells, which were significantly stronger than IMAB362. CS1 and CS2 also had strong killing effect on NUGC4-hCLDN18.2 cells, which are significantly stronger than IMAB362. However, the anti-CD3 monoclonal antibody did not show killing effect in the same experiment system. According to the same experimental procedures, other bispecific antibodies such as Y17, Y18, CS6, CS7, CS8, CS9, CS10 and CS11 of the present invention had an EC₅₀ value of less than 10 pM for killing effect on NUGC4-hCLDN18.2 cells; Y17, Y18, CS7, CS8, CS10 and CS11 had an EC₅₀ value of less than 10 pM for killing effect on NUGC4 cells.

TABLE 5
In vitro killing effect of antibody-mediated hPBMC
on NUGC4 cells and NUGC4-hCLDN18.2 cells

| Bispecific | EC₅₀ (pM) | |
|---|---|---|
| antibodies | NUGC4 | NUGC4~hCLDN18.2 |
| Y1 | No significant killing effect | No significant killing effect |
| Y2 | No significant killing effect | Not reach plateau |
| Y4 | 136.6 | 0.3781 |
| Y6 | 7.993 | 1.070 |
| Y7 | 5.833 | 0.6550 |
| Y8 | 57.83 | 4.088 |
| CS1 | Not reach plateau | 428.4 |
| CS2 | Not reach plateau | 2.170 |
| CS3 | 0.3326 | 0.6984 |
| CS4 | 0.9397 | 0.3064 |
| CS5 | 433.2 | 132.5 |

### 3. In Vitro Killing Effect of Bispecific Antibody on Non-Target Cells

The procedures in 6.2 were repeated using hPBMC obtained by isolation as effector cells and CLDN18.1 expressing cells as non-target cells to detect bispecific antibody mediated in vitro killing effect. As shown in FIG. 8, Y1, Y2, Y4, Y6, Y7, Y8, CS2, CS4 and CS5 all did not have significant killing effect on non-target cells 293T-hCLDN18.1. Under the same experimental conditions, other bispecific antibodies such as Y17, Y18, CS1, CS3, CS6, CS7, CS8, CS9, CS10 and CS11 of the present invention did not show significant killing effect on non-target cells 293T-hCLDN18.1.

### Example 7: Bispecific Antibody-Mediated Activation on Immune Cells

### 1. Activation on Immune Cells

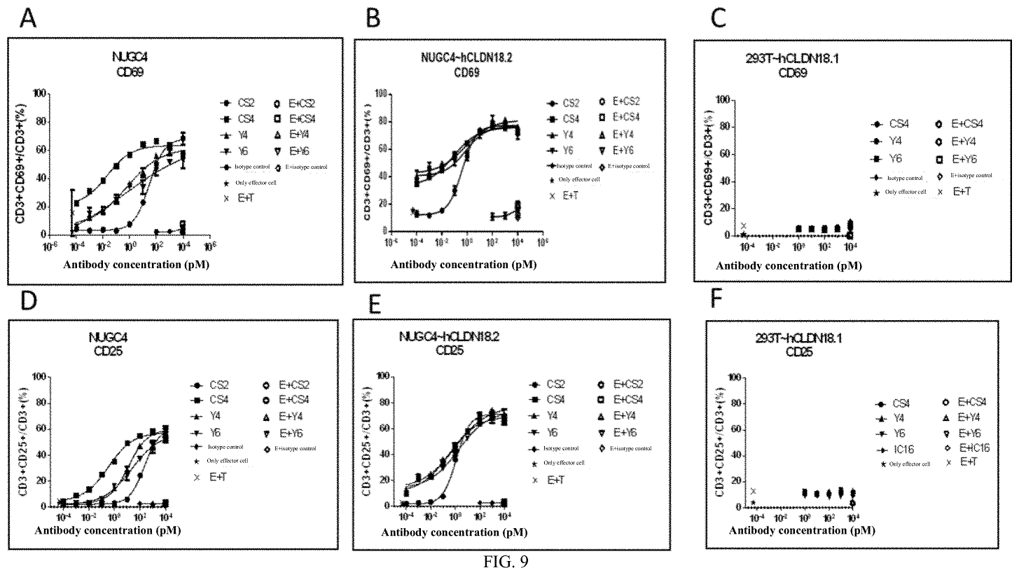

The isolated human peripheral blood mononuclear cells were taken out of the incubator, and digested into a single cell suspension by using pancreatin; 1 mL of the single cell suspension was taken and counted on a VI-Cell counter, and added into a 96-well plate at 2×10⁴ cells/100 μL/well according to the experimental design. The prepared 4×antibody molecules were added at 50 μL/well. The hPBMC was counted on a Cellometer cell counter and then placed in a 96-well plate at 2×10⁵ cells/50 μL/well with an effector-to-target ratio of 10:1; the cell culture plate was placed in a cell incubator for culturing for 48 h. The cell suspension in the cell culture plate was mixed and transferred to another 96-well plate, and after centrifugation at 400×g for 5 min, the hPBMC was resuspended in 100 μL buffer (containing 1.5 μL FITC-labeled anti-CD3 antibody (Biolegend, 300306); 1.5 μL APC-labeled anti-CD25 antibody (Biolegend, 302610) and 1.5 μL PE-labeled anti-CD69 antibody (Biolegend, 310906)). After incubation at 4° C. for 30 min, the cells were washed once with buffer and resuspended in 100 μL buffer, and detected by flow cytometry (BD Accuri™ C6). As shown in FIG. 9 and Table 6, in the presence of the target cells NUGC4, Y4, Y6, Y7, Y8, CS2, CS3, CS4 and CS5 could activate the expression of CD69 molecules (with an EC₅₀ value in a range of 0.4460-140.6 pM) and CD25 molecules (with an EC₅₀ value in a range of 2.377-480.8 pM) on the surface of CD3+T cells; in the presence of target cells NUGC4-hCLDN18.2, Y4, Y6, Y7, Y8, CS2, CS3, CS4 and CS5 could activate the expression of CD69 molecules (with an EC₅₀ value in a range of 0.1956-70.47 pM) and CD25 molecules (with an EC₅₀ value in a range of 0.9623-142.3 pM) on the surface of CD3+T cells; bispecific antibodies had no activation effect on T cells in the presence of the non-target cells 293T-hCLDN18.1. The above results indicated the targeting activation of the bispecific antibodies. According to the same experimental procedures, other bispecific antibodies such as Y17, Y18, CS1, CS6, CS7, CS8, CS9, CS10 and CS11 of the present invention all had good activation effect on T cells in the presence of target cells, and no activation effect on T cells in the absence of target cells.

TABLE 6
Activation of bispecific antibodies on immune cells in the presence of gastric
cancer cells NUGC4, NUGC4-hCLDN18.2 and non-target cells 293T-hCLDN18.1

| | Induction of expression of CD69 | | | Induction of expression of CD25 | | |
|---|---|---|---|---|---|---|
| | EC₅₀ (pM) | | | EC₅₀ (pM) | | |
| Bispecific | | NUGC4~ | 293T~ | | NUGC4~ | 293T~ |
| antibodies | NUGC4 | hCLDN18.2 | hCLDN18.1 | NUGC4 | hCLDN18.2 | hCLDN18.1 |
| Y4 | 0.6097 | 0.6698 | n/a | 12.23 | 0.9623 | n/a |
| Y6 | 4.865 | 0.5916 | n/a | 17.22 | 2.318 | n/a |
| Y7 | 4.133 | 0.4647 | n/a | 15.71 | 1.782 | n/a |
| Y8 | 33.27 | 2.981 | n/a | 181.9 | 16.52 | n/a |
| CS2 | 29.52 | 0.3892 | n/a | 215.5 | 1.210 | n/a |
| CS3 | 0.4614 | 1.448 | n/a | 5.384 | 5.447 | n/a |
| CS4 | 0.4460 | 0.1956 | n/a | 2.377 | 1.189 | n/a |
| CS5 | 140.6 | 70.47 | n/a | 480.8 | 142.3 | n/a |

Note:
n/a represents no significant killing effect.

### 2. Proliferation Effect on Immune Cells

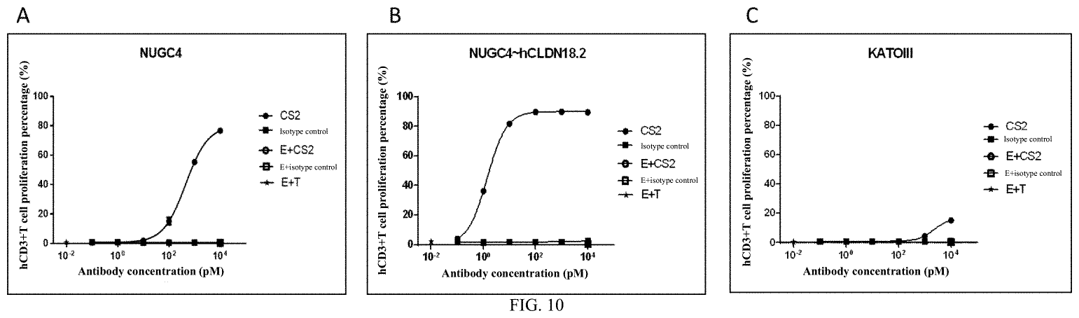

The isolated human peripheral blood mononuclear cells were taken out of the incubator, and digested into a single cell suspension by using pancreatin; 1 mL of the single cell suspension was taken and counted on a VI-Cell counter, and added into a 96-well plate at 2×10⁴ cells/100 μL/well according to the experimental design. The prepared 4×antibody molecules were added at 50 μL/well. The hPBMC cells were stained with 5 μM of CFSE (37° C., 15 min), washed twice with complete medium and counted on a Cellometer cell counter; then the cells were added to a 96-well plate at 2×10⁵ cells/50 μL/well with an effector-to-target ratio of 10:1 according to the experimental design. The cell culture plate was placed in a cell incubator for culturing for 5 days. The cell suspension in the cell culture plate was mixed well, transferred to another 96-well plate, and centrifuged at 400×g for 5 min. The hPBMC was resuspended in 100 μL buffer (containing 1.5 μL of the APC-labeled anti-CD3 antibody) and incubated at 4° C. for 30 min, washed once with buffer, resuspended in 100 μL buffer, and detected by flow cytometry (BD Accuri™ C6). The percentage of the cell population with reduced fluorescence intensity after CFSE staining in CD3+T cell population was analyzed. As shown in FIG. 10, in the presence of target cells NUGC4 and NUGC4-hCLDN18.2, the CS2 bispecific antibody could effectively promote the proliferation effect on CD3+T cells; in the presence of non-target cells KATOIII, only weak proliferation-promoting effect was observed at high dose, which was probably related to weak non-specific adsorption of CS2 molecules to T cells at high dose. Under the same experimental conditions, Y4, Y6, Y7, Y8, CS3 and CS4 could effectively promote the proliferation effect on CD3+T cells in the presence of target cells NUGC4 and NUGC4-hCLDN18.2, while had no T cell proliferation promotion effect in the presence of non-target cells KATOIII. According to the same experimental procedures, other bispecific antibodies such as Y17, Y18, CS1, CS6, CS7, CS8, CS9, CS10 and CS11 all had good proliferation effect on T cells in the presence of target cells, and no proliferation effect on T cells in the absence of target cells.

### Example 8: In Vivo Efficacy of Bispecific Antibodies in Subcutaneous Xenograft Tumors

### 1. In Vivo Efficacy of Bispecific Antibodies in CD3+T Cell Reconstituted Subcutaneous Tumor Model

The in vivo efficacy of bispecific antibodies was evaluated using an immune reconstituted B-NDG mouse NCI-N87-hCLDN18.2 human gastric cancer cell subcutaneous xenograft tumor model (for example, using CS4 bispecific antibody as an example, other antibodies could achieve similar effects according to the experimental procedures). NCI-N87-hCLDN18.2 cell strains are human gastric cancer cell NCI-N87 over-expressing human CLDN18.2 (NCI-N87 purchased from China Center for Type Culture Collection). A sufficient amount of NCI-N87-hCLDN18.2 cells were cultured under culture conditions, and when the cells were in exponential phase, the cells were collected and counted. Each mouse was inoculated subcutaneously at the right anterior scapula with 5×10⁶ human CLDN18.2 overexpression human gastric cancer cells NCI-N87-hCLDN18.2 (suspended in 0.1 mL of PBS). When the tumors grew to 100-200 mm³ (about 10 days after tumor bearing) after inoculation, the tumor-bearing mice were randomly grouped according to the body weight and the tumor volume of the mice, and the experiment was divided into a normal saline group, a CD3⁺ T cell group (5×10⁶ cells/mouse), a CD3⁺ T cell+CS4 (0.2 mg/kg) group (5×10⁶ cells/mouse). Cyclophosphamide was administered for lymphodepletion treatment on the grouping day, and the mice in the test groups were subjected to tail vein injection with CD3+T cells on the next day to reconstruct the immune system of B-NDG. CS4 bispecific antibody intervention was initiated on day 11 after CD3+T cell injection, injected with CS4 twice a week for 6 times. Mice were weighed 2 times weekly, and tumor volumes were measured, and calculated as tumor volume=½×major axis×minor axis×minor axis (mm³). Tumor growth inhibition (TGI) (%)=100%−(Tt−T0)/(Ct−C0)×100%; tumor growth inhibition T/C (%)=(Tt/T0)/(Ct/C0)×100%, wherein TO is average tumor volume of the test antibody group when administered in cages; Tt is average tumor volume of the test antibody group at each measurement; C0 is average tumor volume of the saline group when administered in cages; Ct is average tumor volume of the saline group at each measurement. As shown in FIG. 11 and Table 7, the bispecific antibody CS4 had better tumor inhibition effect in a CD3⁺ T cell immune reconstituted B-NDG mouse subcutaneous NCI-N87-hCLDN18.2 xenograft tumor model, and the tumor growth inhibition (TGI) was 80.11% and the T/C was 24.04% at day 49 after treatment. No significant change of the body weight of the mouse was seen in the treatment process. According to the same experimental procedures, other bispecific antibodies such as Y17, Y18, SY1, CS1, CS6, CS7, CS8, CS9, CS10, CS11 and SCS1 could effectively inhibit tumor growth and were tolerated by the animals.

TABLE 7
In vivo efficacy statistics of the bispecific antibody CS4 in a
CD3⁺T cell immune reconstructed B-NDG mouse subcutaneous
NCI-N87-hCLDN18.2 xenograft tumor model, and statistical
analysis performed according to the tumor volume at day 49

| | | Day 49 | | | |
|---|---|---|---|---|---|
| | | Tumor | | | |
| Group- | | volume (mm³) | TGI | T/C^{a} | P |
| ing | Groups | (mean ± SD) | (%) | (%) | value^{b} |
| Group 1 | Normal saline | 1851.67 ± 583.11 | / | / | / |
| | group | | | | |
| Group 2 | CD3⁺T (5 × 10⁶) | 1822.97 ± 412.26 | 1.67% | 97.82% | 0.9306 |
| Group 3 | CD3⁺T(5 × 10⁶) + | 444.80 ± 108.29 | 80.11% | 24.04% | 0.0007 |
| | CS4 | | | | |

Note:
^{a}T/C is less than or equal to 40%, and p <0.05 is effective after statistical treatment;
^{b}P value is calculated by comparing the data of the group with the saline group.

2. In Vivo Efficacy of Bispecific Antibodies in hCD3E Mouse Subcutaneous Tumor Model

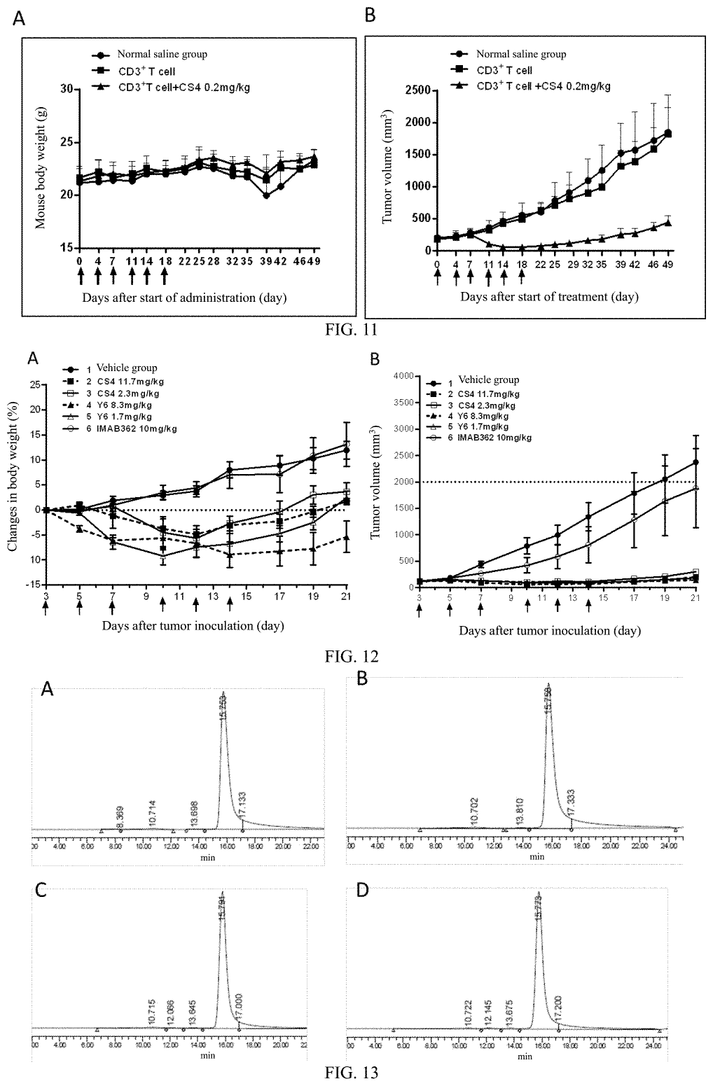

The in vivo efficacy of the bispecific antibodies was evaluated by using a mouse CT26-hCLDN18.2 cell subcutaneous tumor model constructed from BALB/cJGpt-Tg(CD3E BAC)/Gpt mice. BALB/cJGpt-Tg(CD3E BAC)/Gpt mice were purchased from Jiangsu GemPharmatech Co., Ltd., and T lymphocytes thereof expressed human CD3c. A sufficient amount of CT26-hCLDN18.2 cells were cultured, and when the cells were in exponential phase, the cells were collected and counted. After 3-7 days of adaptation period, 2×10⁵ CT26-hCLDN18.2 cells were inoculated subcutaneously to each mouse, and the cells were mixed with Matrigel (volume ratio 1:1) before inoculation, and the total volume of inoculation was 100 μL. The day of inoculation was defined SD0. When the average tumor volume was grown to about 100 mm³, the mice were grouped (6 mice/group) and administered intraperitoneally. The specific groups were as follows: a vehicle group: each mouse was injected with 0.005% (v/v) TW80-saline; a CS4 high dose group (11.7 mg/kg), a CS4 low dose group (2.3 mg/kg), a Y6 high dose group (8.3 mg/kg), a Y6 low dose group (1.7 mg/kg) and an IMAB362 group (10 mg/kg). All tested drugs were formulated with 0.005% TW80-saline. The administration was performed at SD3, SD5, SD7, SD10, SD12 and SD14, and the dose was unchanged. The body weight and tumor volume of the mice were measured before and 2-3 times a week after the administration, and the volume was calculated as tumor volume=½×major axis×minor axis×minor axis (mm³). Tumor growth inhibition (TGI) (%)=100%−(Tt−T0)/(Ct−C0)×100%, and tumor growth inhibition T/C (%)=(Tt/T0)/(Ct/C0)×100%, wherein TO is average tumor volume of the test antibody group when administered in cages; Tt is average tumor volume of the test antibody group at each measurement; C0 is average tumor volume of the vehicle group when administered in cages; Ct is average tumor volume of the vehicle group at each measurement. As shown in FIG. 12 and Table 8, although different levels of body weight reduction occurred after both the high and low doses of the test drugs were administered, the body weights of most mice recovered after the fifth administration on SD12, which exhibited a certain toxicity tolerance. The CS4 (11.7 mg/kg and 2.3 mg/kg) group and Y6 (8.3 mg/kg and 1.7 mg/kg) group all showed good tumor inhibition effects, and showed significant difference with the vehicle group. The IMAB362 (10 mg/kg) group did not show significant tumor inhibition in this experimental system. Other bispecific antibodies such as Y17, Y18, SY1, CS1, CS6, CS7, CS8, CS9, CS10, CS11 and SCS1 were also effective in inhibiting tumor growth according to the same experimental procedures, and were significantly superior to IMAB362, while exhibited good safety.

TABLE 8
In vivo efficacy statistics of bispecific antibodies CS4 and
Y6 in an hCD3E mouse CT26-hCLDN18.2 subcutaneous
tumor model, and statistical analysis performed according
to the tumor volume at day 21

| | | Day 21 | | | |
|---|---|---|---|---|---|
| | | Tumor | | | |
| Group- | | volume (mm³) | TGI | T/C^{a} | P |
| ing | Groups | (mean ± SD) | (%) | (%) | value^{b} |
| Group 1 | Vehicle group | 2371.72 ± 1235.68 | / | / | / |
| Group 2 | CS4 11.7 | 201.85 ± 101.37 | 96.05 | 8.91 | 0.0107 |
| | mg/kg | | | | |
| Group 3 | CS4 2.3 | 301.01 ± 112.50 | 91.80 | 12.91 | 0.0136 |
| | mg/kg | | | | |
| Group 4 | Y6 8.3 mg/kg | 142.75 ± 18.95 | 98.85 | 6.10 | 0.0089 |
| Group 5 | Y6 1.7 mg/kg | 178.32 ± 32.56 | 97.25 | 7.65 | 0.0106 |
| Group 6 | IMAB362 | 1880.12 ± 1830.86 | 21.67 | 81.56 | 0.7418 |
| | 10 mg/kg | | | | |

Note:
^{a}T/C is less than or equal to 40%, and p <0.05 is effective after statistical treatment;
^{b}P value is calculated by comparing the data of the group with the vehicle group.

### Example 9: Evaluation of Acid Stability of Bispecific Antibodies

Bispecific antibodies were evaluated according to a conventional acid stability evaluation method: when the antibody molecules were subjected to protein A affinity chromatography, the eluted antibody solution was not neutralized in the acid elution step (using a citric acid buffer at pH 3.5), and after a certain period of time in this buffer, 1/10 volume of 1 M Tris-HCl (pH 8.0) was added at the 30^{th} min for neutralization and HPLC-SEC detection was performed on this sample. As shown in FIG. 13, the bispecific antibody molecules CS2 and CS4 showed no aggregation or degradation after 30 min of treatment at pH 3.5, and they had a purity of >95%, indicating that the antibody molecules can maintain stability in acidic environment. Other bispecific antibodies Y4, Y6, Y7, Y8, Y17, Y18, CS1, CS3, CS5, CS6, CS7, CS8, CS9, CS10, CS11, SY1 and SCS1 followed the same experimental procedures, showing no aggregation or degradation after 30 min of treatment at pH 3.5, and they had a purity of >95%.

### Example 10: Bispecific Antibodies with Altered Fc Fragments

Fc1 (SEQ ID NO: 20) and Fc2 (SEQ ID NO: 21) in the bispecific antibody in Example 1 were substituted with SEQ ID NO: 22 and SEQ ID NO: 23, respectively, and the sequences of the other portions were unaltered. The expression and purification method and detection method are the same as in Examples 2-8. The results showed that the affinity of the bispecific antibody obtained after Fc substitution for CLDN18.2 and CD3, the in vitro killing effect thereof on CLDN18.2 positive target cells, and the activation effect thereof on immune cells were comparable to those of the bispecific antibody in Example 1. The in vivo efficacy results were slightly stronger than those of the bispecific antibody in Example 1 and had no significant difference.

Both Fc1 (SEQ ID NO: 20) and Fc2 (SEQ ID NO: 21) in the bispecific antibody in Example 1 were substituted with SEQ ID NO: 24, and the sequences of the other portions were unaltered. The expression and purification method and detection method are the same as in Examples 2-8. The results showed that the affinity of the bispecific antibody obtained after Fc substitution for CLDN18.2 and CD3, the in vitro killing effect thereof on CLDN18.2 positive target cells, and the activation effect thereof on immune cells were comparable to those of the bispecific antibody in Example 1. The in vivo efficacy results were slightly stronger than those of the bispecific antibody in Example 1 and had no significant difference.

Both Fc1 (SEQ ID NO: 20) and Fc2 (SEQ ID NO: 21) in the bispecific antibody in Example 1 were substituted with SEQ ID NO: 25, and the sequences of the other portions were unaltered. The expression and purification method and detection method are the same as in Examples 2-8. The results showed that the affinity of the bispecific antibody obtained after Fc substitution for CLDN18.2 and CD3, the in vitro killing effect thereof on CLDN18.2 positive target cells, the activation effect thereof on immune cells and in vivo efficacy thereof were all comparable to those of the bispecific antibody in Example 1, and had no significant difference.

Both Fc1 (SEQ ID NO: 20) and Fc2 (SEQ ID NO: 21) in the bispecific antibody in Example 1 were substituted with SEQ ID NO: 26, and the sequences of the other portions were unaltered. The expression and purification method and detection method are the same as in Examples 2-8. The results showed that the affinity of the bispecific antibody obtained after Fc substitution for CLDN18.2 and CD3, the in vitro killing effect thereof on CLDN18.2 positive target cells, the activation effect thereof on immune cells and in vivo efficacy thereof were all comparable to those of the bispecific antibody in Example 1, and had no significant difference.

### Example 11: Other Bispecific Antibodies

Bispecific antibody molecules YCS2 and YCS4 were constructed according to the method in Example 1. Specifically, the YCS2 was obtained by substituting VHm (SEQ ID NO: 4) of a second heavy chain and VLm (SEQ ID NO: 1) of a second light chain in the bispecific antibody molecule CS2 with the sequences set forth in SEQ ID NO: 6 and SEQ ID NO: 3, respectively, with the sequences of the other portions unaltered. Similarly, the YCS4 was obtained by substituting VHm (SEQ ID NO: 6) of a first heavy chain and VLm (SEQ ID NO: 3) of a first light chain in the bispecific antibody molecule CS4 with the sequences set forth in SEQ ID NO: 5 and SEQ ID NO: 2, respectively, with the sequences of the other portions unaltered. Specific sequences of YCS2 and YCS4 are shown in Table 9 below.

TABLE 9
Sequences of YCS2 and YCS4

| | | | | SEQ |
|---|---|---|---|---|
| Bispecific | | | | ID |
| antibodies | Polypeptide | Domain | No. | NO: |
| YCS2 | First light chain | VLm | 175D10 | 1 |
| | | CL | CL | 18 |
| | Second light chain | VLm | 2C6.9 | 3 |
| | | CL | CL | 18 |
| | Second heavy | VHm | 2C6.9 | 6 |
| | chain | CH1 | CH1 | 19 |
| | | Hinge region 1 | Hin1 | 27 |
| | | Fc1 | G2D1 | 20 |
| | First heavy chain | VHm | 175D10 | 4 |
| | | CH1 | CH1 | 19 |
| | | Hinge region 3 | Hin3 | 29 |
| | | Linker peptide 2 | Lin2 | 31 |
| | | anti-CD3 VH | 2a5 | 10 |
| | | Linker peptide 4 | Lin4 | 8 |
| | | Anti-CD3 VL | 2a5 | 11 |
| | | Linker peptide 3 | Lin3 | 32 |
| | | Hinge region 2 | Hin2 | 28 |
| | | Fc2 | G2D2 | 21 |
| YCS4 | First light chain | VLm | 1E9.2 | 2 |
| | | CL | CL | 18 |
| | Second light chain | VLm | 2C6.9 | 3 |
| | | CL | CL | 18 |
| | Second heavy | VHm | 2C6.9 | 6 |
| | chain | CH1 | CH1 | 19 |
| | | Hinge region 1 | Hin1 | 27 |
| | | Fc1 | G2D1 | 20 |
| | First heavy chain | VHm | 1E9.2 | 5 |
| | | CH1 | CH1 | 19 |
| | | Hinge region 3 | Hin3 | 29 |
| | | Linker peptide 2 | Lin2 | 31 |
| | | anti-CD3 VH | 2a5 | 10 |
| | | Linker peptide 4 | Lin4 | 8 |
| | | Anti-CD3 VL | 2a5 | 11 |
| | | Linker peptide 3 | Lin3 | 32 |
| | | Hinge region 2 | Hin2 | 28 |
| | | Fc2 | G2D2 | 21 |

The expression and purification method and detection method are the same as in Examples 2-8. The results showed that the in vitro killing effect of YCS2 on the tumor cells with low expression of CLDN18.2, the activation effect thereof on immune cells and the in vivo efficacy thereof were superior to that of the bispecific antibody CS2. The binding activity of YCS4 to CLDN18.2 and CD3, the in vitro killing effect thereof on CLDN18.2 positive target cells, the activation effect thereof on immune cells and in vivo efficacy thereof were all comparable to those of bispecific antibody CS4.

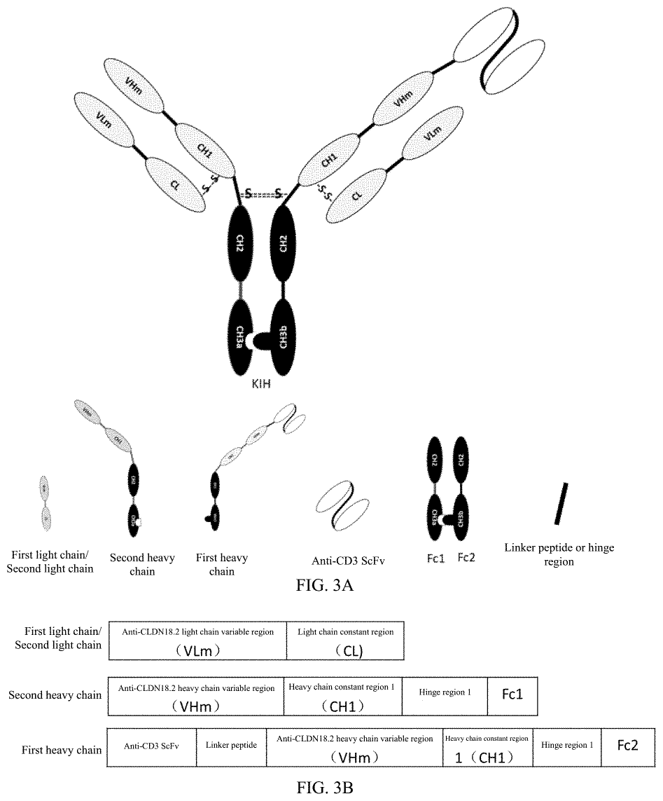

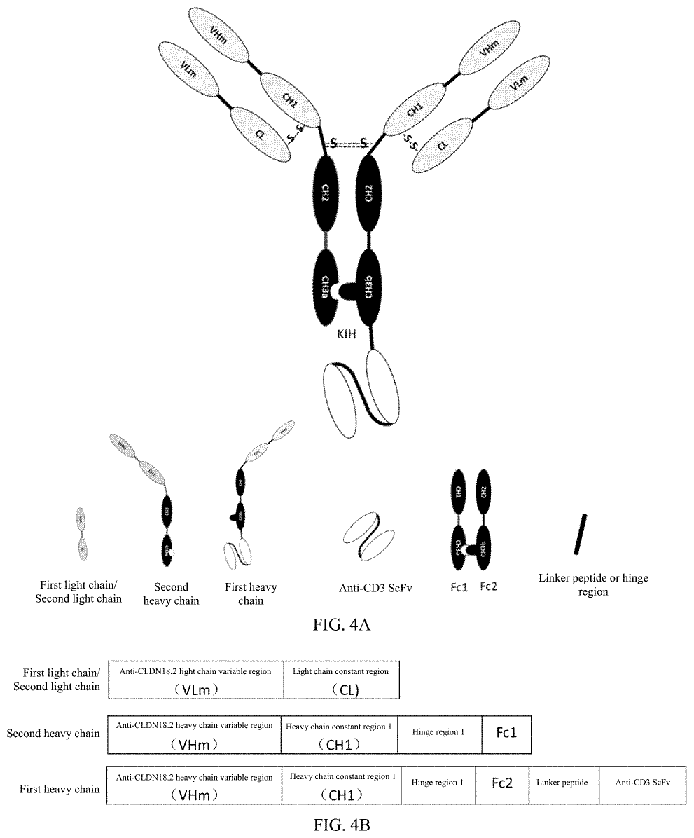

Bispecific antibody NCS3 having a structure shown in FIG. 3 and bispecific antibody CCS3 having a structure shown in FIG. 4 were constructed. The sequences of each portion in NCS3 and CCS3 were identical to those of bispecific antibody CS3, except that the anti-CD3 ScFv was different from linker peptides of VHm or Fc2. In bispecific antibody NCS3, the linker peptide of anti-CD3 ScFv and VHm were linker peptide 2 (SEQ ID NO: 31), and the hinge region between CH1 and FC2 was hinge region 1 (SEQ ID NO: 27). In bispecific antibody CCS3, the linker peptide of anti-CD3 ScFv and VHm was linker peptide 3 (SEQ ID NO: 32), and the hinge region between CH1 and FC2 was hinge region 1 (SEQ ID NO: 27). The expression and purification method and detection method are the same as in Examples 2-8.

The results showed that the affinity of the bispecific antibodies NCS3 and CCS3 for CLDN18.2 and CD3, the in vitro killing effect thereof on CLDN18.2 positive target cells, the activation effect thereof on immune cells and in vivo efficacy thereof were comparable to those of bispecific antibody CS3 and even superior to those of bispecific antibody CS3.

All documents mentioned in the present invention are incorporated by reference, just as each document is cited separately as a reference. In addition, it should be understood that various modifications or changes may be made by those skilled in the art after reading the above teachings of the present invention, and these equivalent forms also fall within the scope defined by the claims appended hereto.

1. A bispecific antibody, comprising an antigen-binding domain specifically binding to CD3 and an antigen-binding domain specifically binding to CLDN18.2,
wherein the antigen-binding domain specifically binding to CD3 is selected from the group consisting of:
1). an antigen-binding domain specifically binding to CD3 comprising the following CDRs or variants thereof:
(i) CDRH1, CDRH2 and CDRH3 comprised in a heavy chain variable region set forth in any one of SEQ ID NO: 7, 10 or 12, and
(ii) CDRL1, CDRL2 and CDRL3 comprised in a light chain variable region set forth in any one of SEQ ID NO: 9, 11 or 13,
preferably, the CDRH1 has a sequence set forth in SEQ ID NO: 79, the CDRH2 has a sequence set forth in SEQ ID NO: 80, the CDRH3 has a sequence set forth in SEQ ID NO: 81 or 85, the CDRL1 has a sequence set forth in SEQ ID NO: 82, the CDRL2 has a sequence set forth in SEQ ID NO: 83, and the CDRL3 has a sequence set forth in SEQ ID NO: 84;
2). an antigen-binding domain specifically binding to CD3 comprising the following CDRs or variants thereof:
(i) CDRH1, CDRH2 and CDRH3 comprised in a heavy chain variable region set forth in SEQ ID NO: 14, and
(ii) CDRL1, CDRL2 and CDRL3 comprised in a light chain variable region set forth in SEQ ID NO: 15,
preferably, the CDRH1 has a sequence set forth in SEQ ID NO: 86, the CDRH2 has a sequence set forth in SEQ ID NO: 87, the CDRH3 has a sequence set forth in SEQ ID NO: 88, the CDRL1 has a sequence set forth in SEQ ID NO: 89, the CDRL2 has a sequence set forth in SEQ ID NO: 90, and the CDRL3 has a sequence set forth in SEQ ID NO: 91; or
3). an antigen-binding domain specifically binding to CD3 comprising the following CDRs or variants thereof:
(i) CDRH1, CDRH2 and CDRH3 comprised in a heavy chain variable region set forth in SEQ ID NO: 16, and
(ii) CDRL1, CDRL2 and CDRL3 comprised in a light chain variable region set forth in SEQ ID NO: 17,
preferably, the CDRH1 has a sequence set forth in SEQ ID NO: 92, the CDRH2 has a sequence set forth in SEQ ID NO: 93, the CDRH3 has a sequence set forth in SEQ ID NO: 94, the CDRL1 has a sequence set forth in SEQ ID NO: 95, the CDRL2 has a sequence set forth in SEQ ID NO: 96, and the CDRL3 has a sequence set forth in SEQ ID NO: 97; and
wherein the antigen-binding domain specifically binding to CLDN18.2 is selected from the group consisting of:
1). an antigen-binding domain specifically binding to CLDN18.2 comprising the following CDRs or variants thereof:
(i) CDRH1, CDRH2 and CDRH3 comprised in a heavy chain variable region set forth in SEQ ID NO: 4, and
(ii) CDRL1, CDRL2 and CDRL3 comprised in a light chain variable region set forth in SEQ ID NO: 1,
preferably, the CDRH1 has a sequence set forth in SEQ ID NO: 47, the CDRH2 has a sequence set forth in SEQ ID NO: 48, the CDRH3 has a sequence set forth in SEQ ID NO: 49, the CDRL1 has a sequence set forth in SEQ ID NO: 50, the CDRL2 has a sequence set forth in SEQ ID NO: 51, and the CDRL3 has a sequence set forth in SEQ ID NO: 52;
2). an antigen-binding domain specifically binding to CLDN18.2 comprising the following CDRs or variants thereof:
(i) CDRH1, CDRH2 and CDRH3 comprised in a heavy chain variable region set forth in SEQ ID NO: 5, and
(ii) CDRL1, CDRL2 and CDRL3 comprised in a light chain variable region set forth in SEQ ID NO: 2,
preferably, the CDRH1 is selected from SEQ ID NOs: 53, 59 and 64, the CDRH2 is selected from SEQ ID NOs: 54, 60 and 65, the CDRH3 is selected from SEQ ID NOs: 55 and 61, the CDRL1 is selected from SEQ ID NOs: 56 and 62, the CDRL2 is selected from SEQ ID NOs: 57 and 63, and the CDRL3 is set forth in SEQ ID NO: 58;
more preferably, the CDRH1 has a sequence set forth in SEQ ID NO: 53, the CDRH2 has a sequence set forth in SEQ ID NO: 54, the CDRH3 has a sequence set forth in SEQ ID NO: 55, the CDRL1 has a sequence set forth in SEQ ID NO: 56, the CDRL2 has a sequence set forth in SEQ ID NO: 57, and the CDRL3 has a sequence set forth in SEQ ID NO: 58;
more preferably, the CDRH1 has a sequence set forth in SEQ ID NO: 59, the CDRH2 has a sequence set forth in SEQ ID NO: 60, the CDRH3 has a sequence set forth in SEQ ID NO: 61, the CDRL1 has a sequence set forth in SEQ ID NO: 62, the CDRL2 has a sequence set forth in SEQ ID NO: 63, and the CDRL3 has a sequence set forth in SEQ ID NO: 58;
more preferably, the CDRH1 has a sequence set forth in SEQ ID NO: 64, the CDRH2 has a sequence set forth in SEQ ID NO: 65, the CDRH3 has a sequence set forth in SEQ ID NO: 61, the CDRL1 has a sequence set forth in SEQ ID NO: 62, the CDRL2 has a sequence set forth in SEQ ID NO: 63, and the CDRL3 has a sequence set forth in SEQ ID NO: 58; or
3). an antigen-binding domain specifically binding to CLDN18.2 comprising the following CDRs or variants thereof:
(i) CDRH1, CDRH2 and CDRH3 comprised in a heavy chain variable region set forth in SEQ ID NO: 6, and
(ii) CDRL1, CDRL2 and CDRL3 comprised in a light chain variable region set forth in SEQ ID NO: 3,
preferably, the CDRH1 is selected from SEQ ID NOs: 66, 72 and 77, the CDRH2 is selected from SEQ ID NOs: 67, 73 and 78, the CDRH3 is selected from SEQ ID NOs: 68 and 74, the CDRL1 is selected from SEQ ID NOs: 69 and 75, the CDRL2 is selected from SEQ ID NOs: 70 and 76, and the CDRL3 is set forth in SEQ ID NO: 71;
more preferably, the CDRH1 has a sequence set forth in SEQ ID NO: 66, the CDRH2 has a sequence set forth in SEQ ID NO: 67, the CDRH3 has a sequence set forth in SEQ ID NO: 68, the CDRL1 has a sequence set forth in SEQ ID NO: 69, the CDRL2 has a sequence set forth in SEQ ID NO: 70, and the CDRL3 has a sequence set forth in SEQ ID NO: 71;
more preferably, the CDRH1 has a sequence set forth in SEQ ID NO: 72, the CDRH2 has a sequence set forth in SEQ ID NO: 73, the CDRH3 has a sequence set forth in SEQ ID NO: 74, the CDRL1 has a sequence set forth in SEQ ID NO: 75, the CDRL2 has a sequence set forth in SEQ ID NO: 76, and the CDRL3 has a sequence set forth in SEQ ID NO: 71;
more preferably, the CDRH1 has a sequence set forth in SEQ ID NO: 77, the CDRH2 has a sequence set forth in SEQ ID NO: 78, the CDRH3 has a sequence set forth in SEQ ID NO: 74, the CDRL1 has a sequence set forth in SEQ ID NO: 75, the CDRL2 has a sequence set forth in SEQ ID NO: 76, and the CDRL3 has a sequence set forth in SEQ ID NO: 71,
wherein the variants differ from the CDRs by 3, 2 or 1 amino acids or have at least 80%, 85%, 90%, 91%, 92%, 93%, 94%, 95%, 96%, 97%, 98% or 99% identity thereto.

2. The bispecific antibody according to claim 1, comprising an antigen-binding domain specifically binding to CD3 and an antigen-binding domain specifically binding to CLDN18.2,
wherein the antigen-binding domain specifically binding to CLDN18.2 comprises the following CDRs (or variants thereof) or variable regions (or variants thereof):
(i) CDRH1, CDRH2 and CDRH3 comprised in a heavy chain variable region set forth in SEQ ID NO: 4, or a heavy chain variable region set forth in SEQ ID NO: 4, and
(ii) CDRL1, CDRL2 and CDRL3 comprised in a light chain variable region set forth in SEQ ID NO: 1, or
a light chain variable region set forth in SEQ ID NO: 1,
preferably, the CDRH1 has a sequence set forth in SEQ ID NO: 47, the CDRH2 has a sequence set forth in SEQ ID NO: 48, the CDRH3 has a sequence set forth in SEQ ID NO: 49, the CDRL1 has a sequence set forth in SEQ ID NO: 50, the CDRL2 has a sequence set forth in SEQ ID NO: 51, and the CDRL3 has a sequence set forth in SEQ ID NO: 52; and
wherein the antigen-binding domain specifically binding to CD3 is selected from the group consisting of:
1). an antigen-binding domain specifically binding to CD3 comprising the following CDRs (or variants thereof) or variable regions (or variants thereof):
(i) CDRH1, CDRH2 and CDRH3 comprised in a heavy chain variable region set forth in SEQ ID NO: 7, or
a heavy chain variable region set forth in SEQ ID NO: 7, and
(ii) CDRL1, CDRL2 and CDRL3 comprised in a light chain variable region set forth in SEQ ID NO: 9, or
a light chain variable region set forth in SEQ ID NO: 9,
preferably, the CDRH1 has a sequence set forth in SEQ ID NO: 79, the CDRH2 has a sequence set forth in SEQ ID NO: 80, the CDRH3 has a sequence set forth in SEQ ID NO: 81, the CDRL1 has a sequence set forth in SEQ ID NO: 82, the CDRL2 has a sequence set forth in SEQ ID NO: 83, and the CDRL3 has a sequence set forth in SEQ ID NO: 84;
2). an antigen-binding domain specifically binding to CD3 comprising the following CDRs (or variants thereof) or variable regions (or variants thereof):
(i) CDRH1, CDRH2 and CDRH3 comprised in a heavy chain variable region set forth in SEQ ID NO: 10, or
a heavy chain variable region set forth in SEQ ID NO: 10, and
(ii) CDRL1, CDRL2 and CDRL3 comprised in a light chain variable region set forth in SEQ ID NO: 11, or
a light chain variable region set forth in SEQ ID NO: 11,
preferably, the CDRH1 has a sequence set forth in SEQ ID NO: 79, the CDRH2 has a sequence set forth in SEQ ID NO: 80, the CDRH3 has a sequence set forth in SEQ ID NO: 85, the CDRL1 has a sequence set forth in SEQ ID NO: 82, the CDRL2 has a sequence set forth in SEQ ID NO: 83, and the CDRL3 has a sequence set forth in SEQ ID NO: 84;
3). an antigen-binding domain specifically binding to CD3 comprising the following CDRs (or variants thereof) or variable regions (or variants thereof):
(i) CDRH1, CDRH2 and CDRH3 comprised in a heavy chain variable region set forth in SEQ ID NO: 12, or
a heavy chain variable region set forth in SEQ ID NO: 12, and
(ii) CDRL1, CDRL2 and CDRL3 comprised in a light chain variable region set forth in SEQ ID NO: 13, or
a light chain variable region set forth in SEQ ID NO: 13,
preferably, the CDRH1 has a sequence set forth in SEQ ID NO: 79, the CDRH2 has a sequence set forth in SEQ ID NO: 80, the CDRH3 has a sequence set forth in SEQ ID NO: 85, the CDRL1 has a sequence set forth in SEQ ID NO: 82, the CDRL2 has a sequence set forth in SEQ ID NO: 83, and the CDRL3 has a sequence set forth in SEQ ID NO: 84;
4). an antigen-binding domain specifically binding to CD3 comprising the following CDRs (or variants thereof) or variable regions (or variants thereof):
(i) CDRH1, CDRH2 and CDRH3 comprised in a heavy chain variable region set forth in SEQ ID NO: 14, or
a heavy chain variable region set forth in SEQ ID NO: 14, and
(ii) CDRL1, CDRL2 and CDRL3 comprised in a light chain variable region set forth in SEQ ID NO: 15, or
a light chain variable region set forth in SEQ ID NO: 15,
preferably, the CDRH1 has a sequence set forth in SEQ ID NO: 86, the CDRH2 has a sequence set forth in SEQ ID NO: 87, the CDRH3 has a sequence set forth in SEQ ID NO: 88, the CDRL1 has a sequence set forth in SEQ ID NO: 89, the CDRL2 has a sequence set forth in SEQ ID NO: 90, and the CDRL3 has a sequence set forth in SEQ ID NO: 91; or
5). an antigen-binding domain specifically binding to CD3 comprising the following CDRs (or variants thereof) or variable regions (or variants thereof):
(i) CDRH1, CDRH2 and CDRH3 comprised in a heavy chain variable region set forth in SEQ ID NO: 16, or
a heavy chain variable region set forth in SEQ ID NO: 16, and
(ii) CDRL1, CDRL2 and CDRL3 comprised in a light chain variable region set forth in SEQ ID NO: 17, or
a light chain variable region set forth in SEQ ID NO: 17,
preferably, the CDRH1 has a sequence set forth in SEQ ID NO: 92, the CDRH2 has a sequence set forth in SEQ ID NO: 93, the CDRH3 has a sequence set forth in SEQ ID NO: 94, the CDRL1 has a sequence set forth in SEQ ID NO: 95, the CDRL2 has a sequence set forth in SEQ ID NO: 96, and the CDRL3 has a sequence set forth in SEQ ID NO: 97,
wherein the variants differ from the CDRs or the variable regions by 3, 2 or 1 amino acids or have at least 80%, 85%, 90%, 91%, 92%, 93%, 94%, 95%, 96%, 97%, 98% or 99% identity thereto.

3. The bispecific antibody according to claim 1, comprising an antigen-binding domain specifically binding to CD3 and an antigen-binding domain specifically binding to CLDN18.2,
wherein the antigen-binding domain specifically binding to CLDN18.2 comprises the following CDRs (or variants thereof) or variable regions (or variants thereof):
(i) CDRH1, CDRH2 and CDRH3 comprised in a heavy chain variable region set forth in SEQ ID NO: 5, or
a heavy chain variable region set forth in SEQ ID NO: 5, and
(ii) CDRL1, CDRL2 and CDRL3 comprised in a light chain variable region set forth in SEQ ID NO: 2, or
a light chain variable region set forth in SEQ ID NO: 2,
preferably, the CDRH1 is selected from SEQ ID NOs: 53, 59 and 64, the CDRH2 is selected from SEQ ID NOs: 54, 60 and 65, the CDRH3 is selected from SEQ ID NOs: 55 and 61, the CDRL1 is selected from SEQ ID NOs: 56 and 62, the CDRL2 is selected from SEQ ID NOs: 57 and 63, and the CDRL3 is set forth in SEQ ID NO: 58;
more preferably, the CDRH1 has a sequence set forth in SEQ ID NO: 53, the CDRH2 has a sequence set forth in SEQ ID NO: 54, the CDRH3 has a sequence set forth in SEQ ID NO: 55, the CDRL1 has a sequence set forth in SEQ ID NO: 56, the CDRL2 has a sequence set forth in SEQ ID NO: 57, and the CDRL3 has a sequence set forth in SEQ ID NO: 58;
more preferably, the CDRH1 has a sequence set forth in SEQ ID NO: 59, the CDRH2 has a sequence set forth in SEQ ID NO: 60, the CDRH3 has a sequence set forth in SEQ ID NO: 61, the CDRL1 has a sequence set forth in SEQ ID NO: 62, the CDRL2 has a sequence set forth in SEQ ID NO: 63, and the CDRL3 has a sequence set forth in SEQ ID NO: 58;
more preferably, the CDRH1 has a sequence set forth in SEQ ID NO: 64, the CDRH2 has a sequence set forth in SEQ ID NO: 65, the CDRH3 has a sequence set forth in SEQ ID NO: 61, the CDRL1 has a sequence set forth in SEQ ID NO: 62, the CDRL2 has a sequence set forth in SEQ ID NO: 63, and the CDRL3 has a sequence set forth in SEQ ID NO: 58; and
wherein the antigen-binding domain specifically binding to CD3 is selected from the group consisting of:
1). an antigen-binding domain specifically binding to CD3 comprising the following CDRs (or variants thereof) or variable regions (or variants thereof):
(i) CDRH1, CDRH2 and CDRH3 comprised in a heavy chain variable region set forth in SEQ ID NO: 7, or
a heavy chain variable region set forth in SEQ ID NO: 7, and
(ii) CDRL1, CDRL2 and CDRL3 comprised in a light chain variable region set forth in SEQ ID NO: 9, or
a light chain variable region set forth in SEQ ID NO: 9,
preferably, the CDRH1 has a sequence set forth in SEQ ID NO: 79, the CDRH2 has a sequence set forth in SEQ ID NO: 80, the CDRH3 has a sequence set forth in SEQ ID NO: 81, the CDRL1 has a sequence set forth in SEQ ID NO: 82, the CDRL2 has a sequence set forth in SEQ ID NO: 83, and the CDRL3 has a sequence set forth in SEQ ID NO: 84;
2). an antigen-binding domain specifically binding to CD3 comprising the following CDRs (or variants thereof) or variable regions (or variants thereof):
(i) CDRH1, CDRH2 and CDRH3 comprised in a heavy chain variable region set forth in SEQ ID NO: 10, or
a heavy chain variable region set forth in SEQ ID NO: 10, and
(ii) CDRL1, CDRL2 and CDRL3 comprised in a light chain variable region set forth in SEQ ID NO: 11, or
a light chain variable region set forth in SEQ ID NO: 11,
preferably, the CDRH1 has a sequence set forth in SEQ ID NO: 79, the CDRH2 has a sequence set forth in SEQ ID NO: 80, the CDRH3 has a sequence set forth in SEQ ID NO: 85, the CDRL1 has a sequence set forth in SEQ ID NO: 82, the CDRL2 has a sequence set forth in SEQ ID NO: 83, and the CDRL3 has a sequence set forth in SEQ ID NO: 84;
3). an antigen-binding domain specifically binding to CD3 comprising the following CDRs (or variants thereof) or variable regions (or variants thereof):
(i) CDRH1, CDRH2 and CDRH3 comprised in a heavy chain variable region set forth in SEQ ID NO: 12, or
a heavy chain variable region set forth in SEQ ID NO: 12, and
(ii) CDRL1, CDRL2 and CDRL3 comprised in a light chain variable region set forth in SEQ ID NO: 13, or
a light chain variable region set forth in SEQ ID NO: 13,
preferably, the CDRH1 has a sequence set forth in SEQ ID NO: 79, the CDRH2 has a sequence set forth in SEQ ID NO: 80, the CDRH3 has a sequence set forth in SEQ ID NO: 85, the CDRL1 has a sequence set forth in SEQ ID NO: 82, the CDRL2 has a sequence set forth in SEQ ID NO: 83, and the CDRL3 has a sequence set forth in SEQ ID NO: 84;
4). an antigen-binding domain specifically binding to CD3 comprising the following CDRs (or variants thereof) or variable regions (or variants thereof):
(i) CDRH1, CDRH2 and CDRH3 comprised in a heavy chain variable region set forth in SEQ ID NO: 14, or
a heavy chain variable region set forth in SEQ ID NO: 14, and
(ii) CDRL1, CDRL2 and CDRL3 comprised in a light chain variable region set forth in SEQ ID NO: 15, or
a light chain variable region set forth in SEQ ID NO: 15,
preferably, the CDRH1 has a sequence set forth in SEQ ID NO: 86, the CDRH2 has a sequence set forth in SEQ ID NO: 87, the CDRH3 has a sequence set forth in SEQ ID NO: 88, the CDRL1 has a sequence set forth in SEQ ID NO: 89, the CDRL2 has a sequence set forth in SEQ ID NO: 90, and the CDRL3 has a sequence set forth in SEQ ID NO: 91; or
5). an antigen-binding domain specifically binding to CD3 comprising the following CDRs (or variants thereof) or variable regions (or variants thereof):
(i) CDRH1, CDRH2 and CDRH3 comprised in a heavy chain variable region set forth in SEQ ID NO: 16, or
a heavy chain variable region set forth in SEQ ID NO: 16, and
(ii) CDRL1, CDRL2 and CDRL3 comprised in a light chain variable region set forth in SEQ ID NO: 17, or
a light chain variable region set forth in SEQ ID NO: 17,
preferably, the CDRH1 has a sequence set forth in SEQ ID NO: 92, the CDRH2 has a sequence set forth in SEQ ID NO: 93, the CDRH3 has a sequence set forth in SEQ ID NO: 94, the CDRL1 has a sequence set forth in SEQ ID NO: 95, the CDRL2 has a sequence set forth in SEQ ID NO: 96, and the CDRL3 has a sequence set forth in SEQ ID NO: 97,
wherein the variants differ from the CDRs or the variable regions by 3, 2 or 1 amino acids or have at least 80%, 85%, 90%, 91%, 92%, 93%, 94%, 95%, 96%, 97%, 98% or 99% identity thereto.

4. The bispecific antibody according to claim 1, comprising an antigen-binding domain specifically binding to CD3 and an antigen-binding domain specifically binding to CLDN18.2,
wherein the antigen-binding domain specifically binding to CLDN18.2 comprises the following CDRs (or variants thereof) or variable regions (or variants thereof):
(i) CDRH1, CDRH2 and CDRH3 comprised in a heavy chain variable region set forth in SEQ ID NO: 6, or
a heavy chain variable region set forth in SEQ ID NO: 6, and
(ii) CDRL1, CDRL2 and CDRL3 comprised in a light chain variable region set forth in SEQ ID NO: 3, or
a light chain variable region set forth in SEQ ID NO: 3,
preferably, the CDRH1 is selected from SEQ ID NOs: 66, 72 and 77, the CDRH2 is selected from SEQ ID NOs: 67, 73 and 78, the CDRH3 is selected from SEQ ID NOs: 68 and 74, the CDRL1 is selected from SEQ ID NOs: 69 and 75, the CDRL2 is selected from SEQ ID NOs: 70 and 76, and the CDRL3 is set forth in SEQ ID NO: 71;
more preferably, the CDRH1 has a sequence set forth in SEQ ID NO: 66, the CDRH2 has a sequence set forth in SEQ ID NO: 67, the CDRH3 has a sequence set forth in SEQ ID NO: 68, the CDRL1 has a sequence set forth in SEQ ID NO: 69, the CDRL2 has a sequence set forth in SEQ ID NO: 70, and the CDRL3 has a sequence set forth in SEQ ID NO: 71;
more preferably, the CDRH1 has a sequence set forth in SEQ ID NO: 72, the CDRH2 has a sequence set forth in SEQ ID NO: 73, the CDRH3 has a sequence set forth in SEQ ID NO: 74, the CDRL1 has a sequence set forth in SEQ ID NO: 75, the CDRL2 has a sequence set forth in SEQ ID NO: 76, and the CDRL3 has a sequence set forth in SEQ ID NO: 71;
more preferably, the CDRH1 has a sequence set forth in SEQ ID NO: 77, the CDRH2 has a sequence set forth in SEQ ID NO: 78, the CDRH3 has a sequence set forth in SEQ ID NO: 74, the CDRL1 has a sequence set forth in SEQ ID NO: 75, the CDRL2 has a sequence set forth in SEQ ID NO: 76, and the CDRL3 has a sequence set forth in SEQ ID NO: 71; and
wherein the antigen-binding domain specifically binding to CD3 is selected from the group consisting of:
1). an antigen-binding domain specifically binding to CD3 comprising the following CDRs (or variants thereof) or variable regions (or variants thereof):
(i) CDRH1, CDRH2 and CDRH3 comprised in a heavy chain variable region set forth in SEQ ID NO: 7, or
a heavy chain variable region set forth in SEQ ID NO: 7, and
(ii) CDRL1, CDRL2 and CDRL3 comprised in a light chain variable region set forth in SEQ ID NO: 9, or
a light chain variable region set forth in SEQ ID NO: 9,
preferably, the CDRH1 has a sequence set forth in SEQ ID NO: 79, the CDRH2 has a sequence set forth in SEQ ID NO: 80, the CDRH3 has a sequence set forth in SEQ ID NO: 81, the CDRL1 has a sequence set forth in SEQ ID NO: 82, the CDRL2 has a sequence set forth in SEQ ID NO: 83, and the CDRL3 has a sequence set forth in SEQ ID NO: 84;
2). an antigen-binding domain specifically binding to CD3 comprising the following CDRs (or variants thereof) or variable regions (or variants thereof):
(i) CDRH1, CDRH2 and CDRH3 comprised in a heavy chain variable region set forth in SEQ ID NO: 10, or
a heavy chain variable region set forth in SEQ ID NO: 10, and
(ii) CDRL1, CDRL2 and CDRL3 comprised in a light chain variable region set forth in SEQ ID NO: 11, or
a light chain variable region set forth in SEQ ID NO: 11,
preferably, the CDRH1 has a sequence set forth in SEQ ID NO: 79, the CDRH2 has a sequence set forth in SEQ ID NO: 80, the CDRH3 has a sequence set forth in SEQ ID NO: 85, the CDRL1 has a sequence set forth in SEQ ID NO: 82, the CDRL2 has a sequence set forth in SEQ ID NO: 83, and the CDRL3 has a sequence set forth in SEQ ID NO: 84;
3). an antigen-binding domain specifically binding to CD3 comprising the following CDRs (or variants thereof) or variable regions (or variants thereof):
(i) CDRH1, CDRH2 and CDRH3 comprised in a heavy chain variable region set forth in SEQ ID NO: 12, or
a heavy chain variable region set forth in SEQ ID NO: 12, and
(ii) CDRL1, CDRL2 and CDRL3 comprised in a light chain variable region set forth in SEQ ID NO: 13, or
a light chain variable region set forth in SEQ ID NO: 13,
preferably, the CDRH1 has a sequence set forth in SEQ ID NO: 79, the CDRH2 has a sequence set forth in SEQ ID NO: 80, the CDRH3 has a sequence set forth in SEQ ID NO: 85, the CDRL1 has a sequence set forth in SEQ ID NO: 82, the CDRL2 has a sequence set forth in SEQ ID NO: 83, and the CDRL3 has a sequence set forth in SEQ ID NO: 84;
4). an antigen-binding domain specifically binding to CD3 comprising the following CDRs (or variants thereof) or variable regions (or variants thereof):
(i) CDRH1, CDRH2 and CDRH3 comprised in a heavy chain variable region set forth in SEQ ID NO: 14, or
a heavy chain variable region set forth in SEQ ID NO: 14, and
(ii) CDRL1, CDRL2 and CDRL3 comprised in a light chain variable region set forth in SEQ ID NO: 15, or
a light chain variable region set forth in SEQ ID NO: 15,
preferably, the CDRH1 has a sequence set forth in SEQ ID NO: 86, the CDRH2 has a sequence set forth in SEQ ID NO: 87, the CDRH3 has a sequence set forth in SEQ ID NO: 88, the CDRL1 has a sequence set forth in SEQ ID NO: 89, the CDRL2 has a sequence set forth in SEQ ID NO: 90, and the CDRL3 has a sequence set forth in SEQ ID NO: 91; or
5). an antigen-binding domain specifically binding to CD3 comprising the following CDRs (or variants thereof) or variable regions (or variants thereof):
(i) CDRH1, CDRH2 and CDRH3 comprised in a heavy chain variable region set forth in SEQ ID NO: 16, or
a heavy chain variable region set forth in SEQ ID NO: 16, and
(ii) CDRL1, CDRL2 and CDRL3 comprised in a light chain variable region set forth in SEQ ID NO: 17, or
a light chain variable region set forth in SEQ ID NO: 17,
preferably, the CDRH1 has a sequence set forth in SEQ ID NO: 92, the CDRH2 has a sequence set forth in SEQ ID NO: 93, the CDRH3 has a sequence set forth in SEQ ID NO: 94, the CDRL1 has a sequence set forth in SEQ ID NO: 95, the CDRL2 has a sequence set forth in SEQ ID NO: 96, and the CDRL3 has a sequence set forth in SEQ ID NO: 97,
wherein the variants differ from the CDRs or the variable regions by 3, 2 or 1 amino acids or have at least 80%, 85%, 90%, 91%, 92%, 93%, 94%, 95%, 96%, 97%, 98% or 99% identity thereto.

5. The bispecific antibody according to claim 1, comprising an antigen-binding domain specifically binding to CD3 and an antigen-binding domain specifically binding to CLDN18.2, wherein
(1) the antigen-binding domain specifically binding to CD3 comprises the following CDRs or variants thereof:
(i) CDRH1, CDRH2 and CDRH3 comprised in a heavy chain variable region set forth in SEQ ID NO: 7, and
(ii) CDRL1, CDRL2 and CDRL3 comprised in a light chain variable region set forth in SEQ ID NO: 9,
preferably, the CDRH1 has a sequence set forth in SEQ ID NO: 79, the CDRH2 has a sequence set forth in SEQ ID NO: 80, the CDRH3 has a sequence set forth in SEQ ID NO: 81, the CDRL1 has a sequence set forth in SEQ ID NO: 82, the CDRL2 has a sequence set forth in SEQ ID NO: 83, and the CDRL3 has a sequence set forth in SEQ ID NO: 84; and
the antigen-binding domain specifically binding to CLDN18.2 comprises the following CDRs or variants thereof:
(i) CDRH1, CDRH2 and CDRH3 comprised in a heavy chain variable region set forth in SEQ ID NO: 4, and
(ii) CDRL1, CDRL2 and CDRL3 comprised in a light chain variable region set forth in SEQ ID NO: 1,
preferably, the CDRH1 has a sequence set forth in SEQ ID NO: 47, the CDRH2 has a sequence set forth in SEQ ID NO: 48, the CDRH3 has a sequence set forth in SEQ ID NO: 49, the CDRL1 has a sequence set forth in SEQ ID NO: 50, the CDRL2 has a sequence set forth in SEQ ID NO: 51, and the CDRL3 has a sequence set forth in SEQ ID NO: 52;
or
(2) the antigen-binding domain specifically binding to CD3 comprises the following CDRs or variants thereof:
(i) CDRH1, CDRH2 and CDRH3 comprised in a heavy chain variable region set forth in SEQ ID NO: 10, and
(ii) CDRL1, CDRL2 and CDRL3 comprised in a light chain variable region set forth in SEQ ID NO: 11,
preferably, the CDRH1 has a sequence set forth in SEQ ID NO: 79, the CDRH2 has a sequence set forth in SEQ ID NO: 80, the CDRH3 has a sequence set forth in SEQ ID NO: 85, the CDRL1 has a sequence set forth in SEQ ID NO: 82, the CDRL2 has a sequence set forth in SEQ ID NO: 83, and the CDRL3 has a sequence set forth in SEQ ID NO: 84; and
the antigen-binding domain specifically binding to CLDN18.2 comprises the following CDRs or variants thereof:
(i) CDRH1, CDRH2 and CDRH3 comprised in a heavy chain variable region set forth in SEQ ID NO: 4, and
(ii) CDRL1, CDRL2 and CDRL3 comprised in a light chain variable region set forth in SEQ ID NO: 1,
preferably, the CDRH1 has a sequence set forth in SEQ ID NO: 47, the CDRH2 has a sequence set forth in SEQ ID NO: 48, the CDRH3 has a sequence set forth in SEQ ID NO: 49, the CDRL1 has a sequence set forth in SEQ ID NO: 50, the CDRL2 has a sequence set forth in SEQ ID NO: 51, and the CDRL3 has a sequence set forth in SEQ ID NO: 52;
or
(3) the antigen-binding domain specifically binding to CD3 comprises the following CDRs or variants thereof:
(i) CDRH1, CDRH2 and CDRH3 comprised in a heavy chain variable region set forth in SEQ ID NO: 12, and
(ii) CDRL1, CDRL2 and CDRL3 comprised in a light chain variable region set forth in SEQ ID NO: 13,
preferably, the CDRH1 has a sequence set forth in SEQ ID NO: 79, the CDRH2 has a sequence set forth in SEQ ID NO: 80, the CDRH3 has a sequence set forth in SEQ ID NO: 85, the CDRL1 has a sequence set forth in SEQ ID NO: 82, the CDRL2 has a sequence set forth in SEQ ID NO: 83, and the CDRL3 has a sequence set forth in SEQ ID NO: 84; and
the antigen-binding domain specifically binding to CLDN18.2 comprises the following CDRs or variants thereof:
(i) CDRH1, CDRH2 and CDRH3 comprised in a heavy chain variable region set forth in SEQ ID NO: 4, and
(ii) CDRL1, CDRL2 and CDRL3 comprised in a light chain variable region set forth in SEQ ID NO: 1,
preferably, the CDRH1 has a sequence set forth in SEQ ID NO: 47, the CDRH2 has a sequence set forth in SEQ ID NO: 48, the CDRH3 has a sequence set forth in SEQ ID NO: 49, the CDRL1 has a sequence set forth in SEQ ID NO: 50, the CDRL2 has a sequence set forth in SEQ ID NO: 51, and the CDRL3 has a sequence set forth in SEQ ID NO: 52;
or
(4) the antigen-binding domain specifically binding to CD3 comprises the following CDRs or variants thereof:
(i) CDRH1, CDRH2 and CDRH3 comprised in a heavy chain variable region set forth in SEQ ID NO: 14, and
(ii) CDRL1, CDRL2 and CDRL3 comprised in a light chain variable region set forth in SEQ ID NO: 15,
preferably, the CDRH1 has a sequence set forth in SEQ ID NO: 86, the CDRH2 has a sequence set forth in SEQ ID NO: 87, the CDRH3 has a sequence set forth in SEQ ID NO: 88, the CDRL1 has a sequence set forth in SEQ ID NO: 89, the CDRL2 has a sequence set forth in SEQ ID NO: 90, and the CDRL3 has a sequence set forth in SEQ ID NO: 91; and
the antigen-binding domain specifically binding to CLDN18.2 comprises the following CDRs or variants thereof:
(i) CDRH1, CDRH2 and CDRH3 comprised in a heavy chain variable region set forth in SEQ ID NO: 4, and
(ii) CDRL1, CDRL2 and CDRL3 comprised in a light chain variable region set forth in SEQ ID NO: 1,
preferably, the CDRH1 has a sequence set forth in SEQ ID NO: 47, the CDRH2 has a sequence set forth in SEQ ID NO: 48, the CDRH3 has a sequence set forth in SEQ ID NO: 49, the CDRL1 has a sequence set forth in SEQ ID NO: 50, the CDRL2 has a sequence set forth in SEQ ID NO: 51, and the CDRL3 has a sequence set forth in SEQ ID NO: 52;
or
(5) the antigen-binding domain specifically binding to CD3 comprises the following CDRs or variants thereof:
(i) CDRH1, CDRH2 and CDRH3 comprised in a heavy chain variable region set forth in SEQ ID NO: 16, and
(ii) CDRL1, CDRL2 and CDRL3 comprised in a light chain variable region set forth in SEQ ID NO: 17,
preferably, the CDRH1 has a sequence set forth in SEQ ID NO: 92, the CDRH2 has a sequence set forth in SEQ ID NO: 93, the CDRH3 has a sequence set forth in SEQ ID NO: 94, the CDRL1 has a sequence set forth in SEQ ID NO: 95, the CDRL2 has a sequence set forth in SEQ ID NO: 96, and the CDRL3 has a sequence set forth in SEQ ID NO: 97; and
the antigen-binding domain specifically binding to CLDN18.2 comprises the following CDRs or variants thereof:
(i) CDRH1, CDRH2 and CDRH3 comprised in a heavy chain variable region set forth in SEQ ID NO: 4, and
(ii) CDRL1, CDRL2 and CDRL3 comprised in a light chain variable region set forth in SEQ ID NO: 1,
preferably, the CDRH1 has a sequence set forth in SEQ ID NO: 47, the CDRH2 has a sequence set forth in SEQ ID NO: 48, the CDRH3 has a sequence set forth in SEQ ID NO: 49, the CDRL1 has a sequence set forth in SEQ ID NO: 50, the CDRL2 has a sequence set forth in SEQ ID NO: 51, and the CDRL3 has a sequence set forth in SEQ ID NO: 52;
or
(6) wherein the antigen-binding domain specifically binding to CD3 comprises the following CDRs or variants thereof:
(i) CDRH1, CDRH2 and CDRH3 comprised in a heavy chain variable region set forth in SEQ ID NO: 7, and
(ii) CDRL1, CDRL2 and CDRL3 comprised in a light chain variable region set forth in SEQ ID NO: 9,
preferably, the CDRH1 has a sequence set forth in SEQ ID NO: 79, the CDRH2 has a sequence set forth in SEQ ID NO: 80, the CDRH3 has a sequence set forth in SEQ ID NO: 81, the CDRL1 has a sequence set forth in SEQ ID NO: 82, the CDRL2 has a sequence set forth in SEQ ID NO: 83, and the CDRL3 has a sequence set forth in SEQ ID NO: 84; and
the antigen-binding domain specifically binding to CLDN18.2 comprises the following CDRs or variants thereof:
(i) CDRH1, CDRH2 and CDRH3 comprised in a heavy chain variable region set forth in SEQ ID NO: 5, and
(ii) CDRL1, CDRL2 and CDRL3 comprised in a light chain variable region set forth in SEQ ID NO: 2,
preferably, the CDRH1 is selected from SEQ ID NOs: 53, 59 and 64, the CDRH2 is selected from SEQ ID NOs: 54, 60 and 65, the CDRH3 is selected from SEQ ID NOs: 55 and 61, the CDRL1 is selected from SEQ ID NOs: 56 and 62, the CDRL2 is selected from SEQ ID NOs: 57 and 63, and the CDRL3 is selected from SEQ ID NO: 58;
more preferably, the CDRH1 has a sequence set forth in SEQ ID NO: 53, the CDRH2 has a sequence set forth in SEQ ID NO: 54, the CDRH3 has a sequence set forth in SEQ ID NO: 55, the CDRL1 has a sequence set forth in SEQ ID NO: 56, the CDRL2 has a sequence set forth in SEQ ID NO: 57, and the CDRL3 has a sequence set forth in SEQ ID NO: 58;
more preferably, the CDRH1 has a sequence set forth in SEQ ID NO: 59, the CDRH2 has a sequence set forth in SEQ ID NO: 60, the CDRH3 has a sequence set forth in SEQ ID NO: 61, the CDRL1 has a sequence set forth in SEQ ID NO: 62, the CDRL2 has a sequence set forth in SEQ ID NO: 63, and the CDRL3 has a sequence set forth in SEQ ID NO: 58;
more preferably, the CDRH1 has a sequence set forth in SEQ ID NO: 64, the CDRH2 has a sequence set forth in SEQ ID NO: 65, the CDRH3 has a sequence set forth in SEQ ID NO: 61, the CDRL1 has a sequence set forth in SEQ ID NO: 62, the CDRL2 has a sequence set forth in SEQ ID NO: 63, and the CDRL3 has a sequence set forth in SEQ ID NO: 58;
or
(7) the antigen-binding domain specifically binding to CD3 comprises the following CDRs or variants thereof:
(i) CDRH1, CDRH2 and CDRH3 comprised in a heavy chain variable region set forth in SEQ ID NO: 10, and
(ii) CDRL1, CDRL2 and CDRL3 comprised in a light chain variable region set forth in SEQ ID NO: 11,
preferably, the CDRH1 has a sequence set forth in SEQ ID NO: 79, the CDRH2 has a sequence set forth in SEQ ID NO: 80, the CDRH3 has a sequence set forth in SEQ ID NO: 85, the CDRL1 has a sequence set forth in SEQ ID NO: 82, the CDRL2 has a sequence set forth in SEQ ID NO: 83, and the CDRL3 has a sequence set forth in SEQ ID NO: 84; and
the antigen-binding domain specifically binding to CLDN18.2 comprises the following CDRs or variants thereof:
(i) CDRH1, CDRH2 and CDRH3 comprised in a heavy chain variable region set forth in SEQ ID NO: 5, and
(ii) CDRL1, CDRL2 and CDRL3 comprised in a light chain variable region set forth in SEQ ID NO: 2,
preferably, the CDRH1 is selected from SEQ ID NOs: 53, 59 and 64, the CDRH2 is selected from SEQ ID NOs: 54, 60 and 65, the CDRH3 is selected from SEQ ID NOs: 55 and 61, the CDRL1 is selected from SEQ ID NOs: 56 and 62, the CDRL2 is selected from SEQ ID NOs: 57 and 63, and the CDRL3 is set forth in SEQ ID NO: 58;
more preferably, the CDRH1 has a sequence set forth in SEQ ID NO: 53, the CDRH2 has a sequence set forth in SEQ ID NO: 54, the CDRH3 has a sequence set forth in SEQ ID NO: 55, the CDRL1 has a sequence set forth in SEQ ID NO: 56, the CDRL2 has a sequence set forth in SEQ ID NO: 57, and the CDRL3 has a sequence set forth in SEQ ID NO: 58;
more preferably, the CDRH1 has a sequence set forth in SEQ ID NO: 59, the CDRH2 has a sequence set forth in SEQ ID NO: 60, the CDRH3 has a sequence set forth in SEQ ID NO: 61, the CDRL1 has a sequence set forth in SEQ ID NO: 62, the CDRL2 has a sequence set forth in SEQ ID NO: 63, and the CDRL3 has a sequence set forth in SEQ ID NO: 58;
more preferably, the CDRH1 has a sequence set forth in SEQ ID NO: 64, the CDRH2 has a sequence set forth in SEQ ID NO: 65, the CDRH3 has a sequence set forth in SEQ ID NO: 61, the CDRL1 has a sequence set forth in SEQ ID NO: 62, the CDRL2 has a sequence set forth in SEQ ID NO: 63, and the CDRL3 has a sequence set forth in SEQ ID NO: 58;
or
(8) the antigen-binding domain specifically binding to CD3 comprises the following CDRs or variants thereof:
(i) CDRH1, CDRH2 and CDRH3 comprised in a heavy chain variable region set forth in SEQ ID NO: 12, and
(ii) CDRL1, CDRL2 and CDRL3 comprised in a light chain variable region set forth in SEQ ID NO: 13,
preferably, the CDRH1 has a sequence set forth in SEQ ID NO: 79, the CDRH2 has a sequence set forth in SEQ ID NO: 80, the CDRH3 has a sequence set forth in SEQ ID NO: 85, the CDRL1 has a sequence set forth in SEQ ID NO: 82, the CDRL2 has a sequence set forth in SEQ ID NO: 83, and the CDRL3 has a sequence set forth in SEQ ID NO: 84; and
the antigen-binding domain specifically binding to CLDN18.2 comprises the following CDRs or variants thereof:
(i) CDRH1, CDRH2 and CDRH3 comprised in a heavy chain variable region set forth in SEQ ID NO: 5, and
(ii) CDRL1, CDRL2 and CDRL3 comprised in a light chain variable region set forth in SEQ ID NO: 2,
preferably, the CDRH1 is selected from SEQ ID NOs: 53, 59 and 64, the CDRH2 is selected from SEQ ID NOs: 54, 60 and 65, the CDRH3 is selected from SEQ ID NOs: 55 and 61, the CDRL1 is selected from SEQ ID NOs: 56 and 62, the CDRL2 is selected from SEQ ID NOs: 57 and 63, and the CDRL3 is set forth in SEQ ID NO: 58;
more preferably, the CDRH1 has a sequence set forth in SEQ ID NO: 53, the CDRH2 has a sequence set forth in SEQ ID NO: 54, the CDRH3 has a sequence set forth in SEQ ID NO: 55, the CDRL1 has a sequence set forth in SEQ ID NO: 56, the CDRL2 has a sequence set forth in SEQ ID NO: 57, and the CDRL3 has a sequence set forth in SEQ ID NO: 58;
more preferably, the CDRH1 has a sequence set forth in SEQ ID NO: 59, the CDRH2 has a sequence set forth in SEQ ID NO: 60, the CDRH3 has a sequence set forth in SEQ ID NO: 61, the CDRL1 has a sequence set forth in SEQ ID NO: 62, the CDRL2 has a sequence set forth in SEQ ID NO: 63, and the CDRL3 has a sequence set forth in SEQ ID NO: 58;
more preferably, the CDRH1 has a sequence set forth in SEQ ID NO: 64, the CDRH2 has a sequence set forth in SEQ ID NO: 65, the CDRH3 has a sequence set forth in SEQ ID NO: 61, the CDRL1 has a sequence set forth in SEQ ID NO: 62, the CDRL2 has a sequence set forth in SEQ ID NO: 63, and the CDRL3 has a sequence set forth in SEQ ID NO: 58;
or
(9) the antigen-binding domain specifically binding to CD3 comprises the following CDRs or variants thereof:
(i) CDRH1, CDRH2 and CDRH3 comprised in a heavy chain variable region set forth in SEQ ID NO: 14, and
(ii) CDRL1, CDRL2 and CDRL3 comprised in a light chain variable region set forth in SEQ ID NO: 15,
preferably, the CDRH1 has a sequence set forth in SEQ ID NO: 86, the CDRH2 has a sequence set forth in SEQ ID NO: 87, the CDRH3 has a sequence set forth in SEQ ID NO: 88, the CDRL1 has a sequence set forth in SEQ ID NO: 89, the CDRL2 has a sequence set forth in SEQ ID NO: 90, and the CDRL3 has a sequence set forth in SEQ ID NO: 91; and
the antigen-binding domain specifically binding to CLDN18.2 comprises the following CDRs or variants thereof:
(i) CDRH1, CDRH2 and CDRH3 comprised in a heavy chain variable region set forth in SEQ ID NO: 5, and
(ii) CDRL1, CDRL2 and CDRL3 comprised in a light chain variable region set forth in SEQ ID NO: 2,
preferably, the CDRH1 is selected from SEQ ID NOs: 53, 59 and 64, the CDRH2 is selected from SEQ ID NOs: 54, 60 and 65, the CDRH3 is selected from SEQ ID NOs: 55 and 61, the CDRL1 is selected from SEQ ID NOs: 56 and 62, the CDRL2 is selected from SEQ ID NOs: 57 and 63, and the CDRL3 is set forth in SEQ ID NO: 58;
more preferably, the CDRH1 has a sequence set forth in SEQ ID NO: 53, the CDRH2 has a sequence set forth in SEQ ID NO: 54, the CDRH3 has a sequence set forth in SEQ ID NO: 55, the CDRL1 has a sequence set forth in SEQ ID NO: 56, the CDRL2 has a sequence set forth in SEQ ID NO: 57, and the CDRL3 has a sequence set forth in SEQ ID NO: 58;
more preferably, the CDRH1 has a sequence set forth in SEQ ID NO: 59, the CDRH2 has a sequence set forth in SEQ ID NO: 60, the CDRH3 has a sequence set forth in SEQ ID NO: 61, the CDRL1 has a sequence set forth in SEQ ID NO: 62, the CDRL2 has a sequence set forth in SEQ ID NO: 63, and the CDRL3 has a sequence set forth in SEQ ID NO: 58;
more preferably, the CDRH1 has a sequence set forth in SEQ ID NO: 64, the CDRH2 has a sequence set forth in SEQ ID NO: 65, the CDRH3 has a sequence set forth in SEQ ID NO: 61, the CDRL1 has a sequence set forth in SEQ ID NO: 62, the CDRL2 has a sequence set forth in SEQ ID NO: 63, and the CDRL3 has a sequence set forth in SEQ ID NO: 58;
or
(10) the antigen-binding domain specifically binding to CD3 comprises the following CDRs or variants thereof:
(i) CDRH1, CDRH2 and CDRH3 comprised in a heavy chain variable region set forth in SEQ ID NO: 16, and
(ii) CDRL1, CDRL2 and CDRL3 comprised in a light chain variable region set forth in SEQ ID NO: 17,
preferably, the CDRH1 has a sequence set forth in SEQ ID NO: 92, the CDRH2 has a sequence set forth in SEQ ID NO: 93, the CDRH3 has a sequence set forth in SEQ ID NO: 94, the CDRL1 has a sequence set forth in SEQ ID NO: 95, the CDRL2 has a sequence set forth in SEQ ID NO: 96, and the CDRL3 has a sequence set forth in SEQ ID NO: 97; and
the antigen-binding domain specifically binding to CLDN18.2 comprises the following CDRs or variants thereof:
(i) CDRH1, CDRH2 and CDRH3 comprised in a heavy chain variable region set forth in SEQ ID NO: 5, and
(ii) CDRL1, CDRL2 and CDRL3 comprised in a light chain variable region set forth in SEQ ID NO: 2,
preferably, the CDRH1 is selected from SEQ ID NOs: 53, 59 and 64, the CDRH2 is selected from SEQ ID NOs: 54, 60 and 65, the CDRH3 is selected from SEQ ID NOs: 55 and 61, the CDRL1 is selected from SEQ ID NOs: 56 and 62, the CDRL2 is selected from SEQ ID NOs: 57 and 63, and the CDRL3 is set forth in SEQ ID NO: 58;
more preferably, the CDRH1 has a sequence set forth in SEQ ID NO: 53, the CDRH2 has a sequence set forth in SEQ ID NO: 54, the CDRH3 has a sequence set forth in SEQ ID NO: 55, the CDRL1 has a sequence set forth in SEQ ID NO: 56, the CDRL2 has a sequence set forth in SEQ ID NO: 57, and the CDRL3 has a sequence set forth in SEQ ID NO: 58;
more preferably, the CDRH1 has a sequence set forth in SEQ ID NO: 59, the CDRH2 has a sequence set forth in SEQ ID NO: 60, the CDRH3 has a sequence set forth in SEQ ID NO: 61, the CDRL1 has a sequence set forth in SEQ ID NO: 62, the CDRL2 has a sequence set forth in SEQ ID NO: 63, and the CDRL3 has a sequence set forth in SEQ ID NO: 58;
more preferably, the CDRH1 has a sequence set forth in SEQ ID NO: 64, the CDRH2 has a sequence set forth in SEQ ID NO: 65, the CDRH3 has a sequence set forth in SEQ ID NO: 61, the CDRL1 has a sequence set forth in SEQ ID NO: 62, the CDRL2 has a sequence set forth in SEQ ID NO: 63, and the CDRL3 has a sequence set forth in SEQ ID NO: 58;
or
(11) wherein the antigen-binding domain specifically binding to CD3 comprises the following CDRs or variants thereof:
(i) CDRH1, CDRH2 and CDRH3 comprised in a heavy chain variable region set forth in SEQ ID NO: 7, and
(ii) CDRL1, CDRL2 and CDRL3 comprised in a light chain variable region set forth in SEQ ID NO: 9,
preferably, the CDRH1 has a sequence set forth in SEQ ID NO: 79, the CDRH2 has a sequence set forth in SEQ ID NO: 80, the CDRH3 has a sequence set forth in SEQ ID NO: 81, the CDRL1 has a sequence set forth in SEQ ID NO: 82, the CDRL2 has a sequence set forth in SEQ ID NO: 83, and the CDRL3 has a sequence set forth in SEQ ID NO: 84; and
the antigen-binding domain specifically binding to CLDN18.2 comprises the following CDRs or variants thereof:
(i) CDRH1, CDRH2 and CDRH3 comprised in a heavy chain variable region set forth in SEQ ID NO: 6, and
(ii) CDRL1, CDRL2 and CDRL3 comprised in a light chain variable region set forth in SEQ ID NO: 3,
preferably, the CDRH1 is selected from SEQ ID NOs: 66, 72 and 77, the CDRH2 is selected from SEQ ID NOs: 67, 73 and 78, the CDRH3 is selected from SEQ ID NOs: 68 and 74, the CDRL1 is selected from SEQ ID NOs: 69 and 75, the CDRL2 is selected from SEQ ID NOs: 70 and 76, and the CDRL3 is set forth in SEQ ID NO: 71;
more preferably, the CDRH1 has a sequence set forth in SEQ ID NO: 66, the CDRH2 has a sequence set forth in SEQ ID NO: 67, the CDRH3 has a sequence set forth in SEQ ID NO: 68, the CDRL1 has a sequence set forth in SEQ ID NO: 69, the CDRL2 has a sequence set forth in SEQ ID NO: 70, and the CDRL3 has a sequence set forth in SEQ ID NO: 71;
more preferably, the CDRH1 has a sequence set forth in SEQ ID NO: 72, the CDRH2 has a sequence set forth in SEQ ID NO: 73, the CDRH3 has a sequence set forth in SEQ ID NO: 74, the CDRL1 has a sequence set forth in SEQ ID NO: 75, the CDRL2 has a sequence set forth in SEQ ID NO: 76, and the CDRL3 has a sequence set forth in SEQ ID NO: 71;
more preferably, the CDRH1 has a sequence set forth in SEQ ID NO: 77, the CDRH2 has a sequence set forth in SEQ ID NO: 78, the CDRH3 has a sequence set forth in SEQ ID NO: 74, the CDRL1 has a sequence set forth in SEQ ID NO: 75, the CDRL2 has a sequence set forth in SEQ ID NO: 76, and the CDRL3 has a sequence set forth in SEQ ID NO: 71;
or
(12) the antigen-binding domain specifically binding to CD3 comprises the following CDRs or variants thereof:
(i) CDRH1, CDRH2 and CDRH3 comprised in a heavy chain variable region set forth in SEQ ID NO: 10, and
(ii) CDRL1, CDRL2 and CDRL3 comprised in a light chain variable region set forth in SEQ ID NO: 11,
preferably, the CDRH1 has a sequence set forth in SEQ ID NO: 79, the CDRH2 has a sequence set forth in SEQ ID NO: 80, the CDRH3 has a sequence set forth in SEQ ID NO: 85, the CDRL1 has a sequence set forth in SEQ ID NO: 82, the CDRL2 has a sequence set forth in SEQ ID NO: 83, and the CDRL3 has a sequence set forth in SEQ ID NO: 84; and
the antigen-binding domain specifically binding to CLDN18.2 comprises the following CDRs or variants thereof:
(i) CDRH1, CDRH2 and CDRH3 comprised in a heavy chain variable region set forth in SEQ ID NO: 6, and
(ii) CDRL1, CDRL2 and CDRL3 comprised in a light chain variable region set forth in SEQ ID NO: 3,
preferably, the CDRH1 is selected from SEQ ID NOs: 66, 72 and 77, the CDRH2 is selected from SEQ ID NOs: 67, 73 and 78, the CDRH3 is selected from SEQ ID NOs: 68 and 74, the CDRL1 is selected from SEQ ID NOs: 69 and 75, the CDRL2 is selected from SEQ ID NOs: 70 and 76, and the CDRL3 is set forth in SEQ ID NO: 71;
more preferably, the CDRH1 has a sequence set forth in SEQ ID NO: 66, the CDRH2 has a sequence set forth in SEQ ID NO: 67, the CDRH3 has a sequence set forth in SEQ ID NO: 68, the CDRL1 has a sequence set forth in SEQ ID NO: 69, the CDRL2 has a sequence set forth in SEQ ID NO: 70, and the CDRL3 has a sequence set forth in SEQ ID NO: 71;
more preferably, the CDRH1 has a sequence set forth in SEQ ID NO: 72, the CDRH2 has a sequence set forth in SEQ ID NO: 73, the CDRH3 has a sequence set forth in SEQ ID NO: 74, the CDRL1 has a sequence set forth in SEQ ID NO: 75, the CDRL2 has a sequence set forth in SEQ ID NO: 76, and the CDRL3 has a sequence set forth in SEQ ID NO: 71;
more preferably, the CDRH1 has a sequence set forth in SEQ ID NO: 77, the CDRH2 has a sequence set forth in SEQ ID NO: 78, the CDRH3 has a sequence set forth in SEQ ID NO: 74, the CDRL1 has a sequence set forth in SEQ ID NO: 75, the CDRL2 has a sequence set forth in SEQ ID NO: 76, and the CDRL3 has a sequence set forth in SEQ ID NO: 71;
or
(13) the antigen-binding domain specifically binding to CD3 comprises the following CDRs or variants thereof:
(i) CDRH1, CDRH2 and CDRH3 comprised in a heavy chain variable region set forth in SEQ ID NO: 12, and
(ii) CDRL1, CDRL2 and CDRL3 comprised in a light chain variable region set forth in SEQ ID NO: 13,
preferably, the CDRH1 has a sequence set forth in SEQ ID NO: 79, the CDRH2 has a sequence set forth in SEQ ID NO: 80, the CDRH3 has a sequence set forth in SEQ ID NO: 85, the CDRL1 has a sequence set forth in SEQ ID NO: 82, the CDRL2 has a sequence set forth in SEQ ID NO: 83, and the CDRL3 has a sequence set forth in SEQ ID NO: 84; and
the antigen-binding domain specifically binding to CLDN18.2 comprises the following CDRs or variants thereof:
(i) CDRH1, CDRH2 and CDRH3 comprised in a heavy chain variable region set forth in SEQ ID NO: 6, and
(ii) CDRL1, CDRL2 and CDRL3 comprised in a light chain variable region set forth in SEQ ID NO: 3,
preferably, the CDRH1 is selected from SEQ ID NOs: 66, 72 and 77, the CDRH2 is selected from SEQ ID NOs: 67, 73 and 78, the CDRH3 is selected from SEQ ID NOs: 68 and 74, the CDRL1 is selected from SEQ ID NOs: 69 and 75, the CDRL2 is selected from SEQ ID NOs: 70 and 76, and the CDRL3 is set forth in SEQ ID NO: 71;
more preferably, the CDRH1 has a sequence set forth in SEQ ID NO: 66, the CDRH2 has a sequence set forth in SEQ ID NO: 67, the CDRH3 has a sequence set forth in SEQ ID NO: 68, the CDRL1 has a sequence set forth in SEQ ID NO: 69, the CDRL2 has a sequence set forth in SEQ ID NO: 70, and the CDRL3 has a sequence set forth in SEQ ID NO: 71;
more preferably, the CDRH1 has a sequence set forth in SEQ ID NO: 72, the CDRH2 has a sequence set forth in SEQ ID NO: 73, the CDRH3 has a sequence set forth in SEQ ID NO: 74, the CDRL1 has a sequence set forth in SEQ ID NO: 75, the CDRL2 has a sequence set forth in SEQ ID NO: 76, and the CDRL3 has a sequence set forth in SEQ ID NO: 71;
more preferably, the CDRH1 has a sequence set forth in SEQ ID NO: 77, the CDRH2 has a sequence set forth in SEQ ID NO: 78, the CDRH3 has a sequence set forth in SEQ ID NO: 74, the CDRL1 has a sequence set forth in SEQ ID NO: 75, the CDRL2 has a sequence set forth in SEQ ID NO: 76, and the CDRL3 has a sequence set forth in SEQ ID NO: 71;
or
(14) the antigen-binding domain specifically binding to CD3 comprises the following CDRs or variants thereof:
(i) CDRH1, CDRH2 and CDRH3 comprised in a heavy chain variable region set forth in SEQ ID NO: 14, and
(ii) CDRL1, CDRL2 and CDRL3 comprised in a light chain variable region set forth in SEQ ID NO: 15,
preferably, the CDRH1 has a sequence set forth in SEQ ID NO: 86, the CDRH2 has a sequence set forth in SEQ ID NO: 87, the CDRH3 has a sequence set forth in SEQ ID NO: 88, the CDRL1 has a sequence set forth in SEQ ID NO: 89, the CDRL2 has a sequence set forth in SEQ ID NO: 90, and the CDRL3 has a sequence set forth in SEQ ID NO: 91; and
the antigen-binding domain specifically binding to CLDN18.2 comprises the following CDRs or variants thereof:
(i) CDRH1, CDRH2 and CDRH3 comprised in a heavy chain variable region set forth in SEQ ID NO: 6, and
(ii) CDRL1, CDRL2 and CDRL3 comprised in a light chain variable region set forth in SEQ ID NO: 3,
preferably, the CDRH1 is selected from SEQ ID NOs: 66, 72 and 77, the CDRH2 is selected from SEQ ID NOs: 67, 73 and 78, the CDRH3 is selected from SEQ ID NOs: 68 and 74, the CDRL1 is selected from SEQ ID NOs: 69 and 75, the CDRL2 is selected from SEQ ID NOs: 70 and 76, and the CDRL3 is set forth in SEQ ID NO: 71;
more preferably, the CDRH1 has a sequence set forth in SEQ ID NO: 66, the CDRH2 has a sequence set forth in SEQ ID NO: 67, the CDRH3 has a sequence set forth in SEQ ID NO: 68, the CDRL1 has a sequence set forth in SEQ ID NO: 69, the CDRL2 has a sequence set forth in SEQ ID NO: 70, and the CDRL3 has a sequence set forth in SEQ ID NO: 71;
more preferably, the CDRH1 has a sequence set forth in SEQ ID NO: 72, the CDRH2 has a sequence set forth in SEQ ID NO: 73, the CDRH3 has a sequence set forth in SEQ ID NO: 74, the CDRL1 has a sequence set forth in SEQ ID NO: 75, the CDRL2 has a sequence set forth in SEQ ID NO: 76, and the CDRL3 has a sequence set forth in SEQ ID NO: 71;
more preferably, the CDRH1 has a sequence set forth in SEQ ID NO: 77, the CDRH2 has a sequence set forth in SEQ ID NO: 78, the CDRH3 has a sequence set forth in SEQ ID NO: 74, the CDRL1 has a sequence set forth in SEQ ID NO: 75, the CDRL2 has a sequence set forth in SEQ ID NO: 76, and the CDRL3 has a sequence set forth in SEQ ID NO: 71;
or
(15) the antigen-binding domain specifically binding to CD3 comprises the following CDRs or variants thereof:
(i) CDRH1, CDRH2 and CDRH3 comprised in a heavy chain variable region set forth in SEQ ID NO: 16, and
(ii) CDRL1, CDRL2 and CDRL3 comprised in a light chain variable region set forth in SEQ ID NO: 17,
preferably, the CDRH1 has a sequence set forth in SEQ ID NO: 92, the CDRH2 has a sequence set forth in SEQ ID NO: 93, the CDRH3 has a sequence set forth in SEQ ID NO: 94, the CDRL1 has a sequence set forth in SEQ ID NO: 95, the CDRL2 has a sequence set forth in SEQ ID NO: 96, and the CDRL3 has a sequence set forth in SEQ ID NO: 97; and
the antigen-binding domain specifically binding to CLDN18.2 comprises the following CDRs or variants thereof:
(i) CDRH1, CDRH2 and CDRH3 comprised in a heavy chain variable region set forth in SEQ ID NO: 6, and
(ii) CDRL1, CDRL2 and CDRL3 comprised in a light chain variable region set forth in SEQ ID NO: 3,
preferably, the CDRH1 is selected from SEQ ID NOs: 66, 72 and 77, the CDRH2 is selected from SEQ ID NOs: 67, 73 and 78, the CDRH3 is selected from SEQ ID NOs: 68 and 74, the CDRL1 is selected from SEQ ID NOs: 69 and 75, the CDRL2 is selected from SEQ ID NOs: 70 and 76, and the CDRL3 is set forth in SEQ ID NO: 71;
more preferably, the CDRH1 has a sequence set forth in SEQ ID NO: 66, the CDRH2 has a sequence set forth in SEQ ID NO: 67, the CDRH3 has a sequence set forth in SEQ ID NO: 68, the CDRL1 has a sequence set forth in SEQ ID NO: 69, the CDRL2 has a sequence set forth in SEQ ID NO: 70, and the CDRL3 has a sequence set forth in SEQ ID NO: 71;
more preferably, the CDRH1 has a sequence set forth in SEQ ID NO: 72, the CDRH2 has a sequence set forth in SEQ ID NO: 73, the CDRH3 has a sequence set forth in SEQ ID NO: 74, the CDRL1 has a sequence set forth in SEQ ID NO: 75, the CDRL2 has a sequence set forth in SEQ ID NO: 76, and the CDRL3 has a sequence set forth in SEQ ID NO: 71;
more preferably, the CDRH1 has a sequence set forth in SEQ ID NO: 77, the CDRH2 has a sequence set forth in SEQ ID NO: 78, the CDRH3 has a sequence set forth in SEQ ID NO: 74, the CDRL1 has a sequence set forth in SEQ ID NO: 75, the CDRL2 has a sequence set forth in SEQ ID NO: 76, and the CDRL3 has a sequence set forth in SEQ ID NO: 71;
wherein the variants differ from the CDRs by 3, 2 or 1 amino acids or have at least 80%, 85%, 90%, 91%, 92%, 93%, 94%, 95%, 96%, 97%, 98% or 99% identity thereto.

6. The bispecific antibody according to claim 1, wherein the antigen-binding domain specifically binding to CD3 is selected from the group consisting of:
1). an antigen-binding domain specifically binding to CD3 comprising the following variable region amino acid sequences or variants thereof:
(i) a heavy chain variable region amino acid sequence set forth in SEQ ID NO: 7, and
(ii) a light chain variable region amino acid sequence set forth in SEQ ID NO: 9;
2). an antigen-binding domain specifically binding to CD3 comprising the following variable region amino acid sequences or variants thereof:
(i) a heavy chain variable region amino acid sequence set forth in SEQ ID NO: 10, and
(ii) a light chain variable region amino acid sequence set forth in SEQ ID NO: 11;
3). an antigen-binding domain specifically binding to CD3 comprising the following variable region amino acid sequences or variants thereof:
(i) a heavy chain variable region amino acid sequence set forth in SEQ ID NO: 12, and
(ii) a light chain variable region amino acid sequence set forth in SEQ ID NO: 13;
4). an antigen-binding domain specifically binding to CD3 comprising the following variable region amino acid sequences or variants thereof:
(i) a heavy chain variable region amino acid sequence set forth in SEQ ID NO: 14, and
(ii) a light chain variable region amino acid sequence set forth in SEQ ID NO: 15; or
5). an antigen-binding domain specifically binding to CD3 comprising the following variable region amino acid sequences or variants thereof:
(i) a heavy chain variable region amino acid sequence set forth in SEQ ID NO: 16, and
(ii) a light chain variable region amino acid sequence set forth in SEQ ID NO: 17;
wherein the antigen-binding domain specifically binding to CLDN18.2 is selected from the group consisting of:
1). an antigen-binding domain specifically binding to CLDN18.2 comprising the following variable region amino acid sequences or variants thereof:
(i) a heavy chain variable region amino acid sequence set forth in SEQ ID NO: 4, and
(ii) a light chain variable region amino acid sequence set forth in SEQ ID NO: 1;
2). an antigen-binding domain specifically binding to CLDN18.2 comprising the following variable region amino acid sequences or variants thereof:
(i) a heavy chain variable region amino acid sequence set forth in SEQ ID NO: 5, and
(ii) a light chain variable region amino acid sequence set forth in SEQ ID NO: 2; or
3). an antigen-binding domain specifically binding to CLDN18.2 comprising the following variable region amino acid sequences or variants thereof:
(i) a heavy chain variable region amino acid sequence set forth in SEQ ID NO: 6, and
(ii) a light chain variable region amino acid sequence set forth in SEQ ID NO: 3,
wherein the variants differ from the variable region amino acid sequences by 3, 2 or 1 amino acids or have at least 80%, 85%, 90%, 91%, 92%, 93%, 94%, 95%, 96%, 97%, 98% or 99% identity thereto.

7. The bispecific antibody according to claim 1, comprising an antigen-binding domain specifically binding to CD3 and an antigen-binding domain specifically binding to CLDN18.2, wherein
(1) the antigen-binding domain specifically binding to CD3 comprises the following variable region amino acid sequences or variants thereof:
(i) a heavy chain variable region amino acid sequence set forth in SEQ ID NO: 7, and
(ii) a light chain variable region amino acid sequence set forth in SEQ ID NO: 9; and
the antigen-binding domain specifically binding to CLDN18.2 comprises the following variable region amino acid sequences or variants thereof:
(i) a heavy chain variable region amino acid sequence set forth in SEQ ID NO: 4, and
(ii) a light chain variable region amino acid sequence set forth in SEQ ID NO: 1; or
(2) the antigen-binding domain specifically binding to CD3 comprises the following variable region amino acid sequences or variants thereof:
(i) a heavy chain variable region amino acid sequence set forth in SEQ ID NO: 10, and
(ii) a light chain variable region amino acid sequence set forth in SEQ ID NO: 11; and
the antigen-binding domain specifically binding to CLDN18.2 comprises the following variable region amino acid sequences or variants thereof:
(i) a heavy chain variable region amino acid sequence set forth in SEQ ID NO: 4, and
(ii) a light chain variable region amino acid sequence set forth in SEQ ID NO: 1; or
(3) the antigen-binding domain specifically binding to CD3 comprises the following variable region amino acid sequences or variants thereof:
(i) a heavy chain variable region amino acid sequence set forth in SEQ ID NO: 12, and
(ii) a light chain variable region amino acid sequence set forth in SEQ ID NO: 13; and
the antigen-binding domain specifically binding to CLDN18.2 comprises the following variable region amino acid sequences or variants thereof:
(i) a heavy chain variable region amino acid sequence set forth in SEQ ID NO: 4, and
(ii) a light chain variable region amino acid sequence set forth in SEQ ID NO: 1; or
(4) the antigen-binding domain specifically binding to CD3 comprises the following variable region amino acid sequences or variants thereof:
(i) a heavy chain variable region amino acid sequence set forth in SEQ ID NO: 14, and
(ii) a light chain variable region amino acid sequence set forth in SEQ ID NO: 15; and
the antigen-binding domain specifically binding to CLDN18.2 comprises the following variable region amino acid sequences or variants thereof:
(i) a heavy chain variable region amino acid sequence set forth in SEQ ID NO: 4, and
(ii) a light chain variable region amino acid sequence set forth in SEQ ID NO: 1; or
(5) the antigen-binding domain specifically binding to CD3 comprises the following variable region amino acid sequences or variants thereof:
(i) a heavy chain variable region amino acid sequence set forth in SEQ ID NO: 16, and
(ii) a light chain variable region amino acid sequence set forth in SEQ ID NO: 17; and
the antigen-binding domain specifically binding to CLDN18.2 comprises the following variable region amino acid sequences or variants thereof:
(i) a heavy chain variable region amino acid sequence set forth in SEQ ID NO: 4, and
(ii) a light chain variable region amino acid sequence set forth in SEQ ID NO: 1; or
(6) the antigen-binding domain specifically binding to CD3 comprises the following variable region amino acid sequences or variants thereof:
(i) a heavy chain variable region amino acid sequence set forth in SEQ ID NO: 7, and
(ii) a light chain variable region amino acid sequence set forth in SEQ ID NO: 9; and
the antigen-binding domain specifically binding to CLDN18.2 comprises the following variable region amino acid sequences or variants thereof:
(i) a heavy chain variable region amino acid sequence set forth in SEQ ID NO: 5, and
(ii) a light chain variable region amino acid sequence set forth in SEQ ID NO: 2; or
(7) the antigen-binding domain specifically binding to CD3 comprises the following variable region amino acid sequences or variants thereof:
(i) a heavy chain variable region amino acid sequence set forth in SEQ ID NO: 10, and
(ii) a light chain variable region amino acid sequence set forth in SEQ ID NO: 11; and
the antigen-binding domain specifically binding to CLDN18.2 comprises the following variable region amino acid sequences or variants thereof:
(i) a heavy chain variable region amino acid sequence set forth in SEQ ID NO: 5, and
(ii) a light chain variable region amino acid sequence set forth in SEQ ID NO: 2; or
(8) the antigen-binding domain specifically binding to CD3 comprises the following variable region amino acid sequences or variants thereof:
(i) a heavy chain variable region amino acid sequence set forth in SEQ ID NO: 12, and
(ii) a light chain variable region amino acid sequence set forth in SEQ ID NO: 13; and
the antigen-binding domain specifically binding to CLDN18.2 comprises the following variable region amino acid sequences or variants thereof:
(i) a heavy chain variable region amino acid sequence set forth in SEQ ID NO: 5, and
(ii) a light chain variable region amino acid sequence set forth in SEQ ID NO: 2; or
(9) the antigen-binding domain specifically binding to CD3 comprises the following variable region amino acid sequences or variants thereof:
(i) a heavy chain variable region amino acid sequence set forth in SEQ ID NO: 14, and
(ii) a light chain variable region amino acid sequence set forth in SEQ ID NO: 15; and
the antigen-binding domain specifically binding to CLDN18.2 comprises the following variable region amino acid sequences or variants thereof:
(i) a heavy chain variable region amino acid sequence set forth in SEQ ID NO: 5, and
(ii) a light chain variable region amino acid sequence set forth in SEQ ID NO: 2; or
(10) the antigen-binding domain specifically binding to CD3 comprises the following variable region amino acid sequences or variants thereof:
(i) a heavy chain variable region amino acid sequence set forth in SEQ ID NO: 16, and
(ii) a light chain variable region amino acid sequence set forth in SEQ ID NO: 17; and
the antigen-binding domain specifically binding to CLDN18.2 comprises the following variable region amino acid sequences or variants thereof:
(i) a heavy chain variable region amino acid sequence set forth in SEQ ID NO: 5, and
(ii) a light chain variable region amino acid sequence set forth in SEQ ID NO: 2; or
(11) the antigen-binding domain specifically binding to CD3 comprises the following variable region amino acid sequences or variants thereof:
(i) a heavy chain variable region amino acid sequence set forth in SEQ ID NO: 7, and
(ii) a light chain variable region amino acid sequence set forth in SEQ ID NO: 9; and
the antigen-binding domain specifically binding to CLDN18.2 comprises the following variable region amino acid sequences or variants thereof:
(i) a heavy chain variable region amino acid sequence set forth in SEQ ID NO: 6, and
(ii) a light chain variable region amino acid sequence set forth in SEQ ID NO: 3; or
(12) the antigen-binding domain specifically binding to CD3 comprises the following variable region amino acid sequences or variants thereof:
(i) a heavy chain variable region amino acid sequence set forth in SEQ ID NO: 10, and
(ii) a light chain variable region amino acid sequence set forth in SEQ ID NO: 11; and
the antigen-binding domain specifically binding to CLDN18.2 comprises the following variable region amino acid sequences or variants thereof:
(i) a heavy chain variable region amino acid sequence set forth in SEQ ID NO: 6, and
(ii) a light chain variable region amino acid sequence set forth in SEQ ID NO: 3; or
(13) the antigen-binding domain specifically binding to CD3 comprises the following variable region amino acid sequences or variants thereof:
(i) a heavy chain variable region amino acid sequence set forth in SEQ ID NO: 12, and
(ii) a light chain variable region amino acid sequence set forth in SEQ ID NO: 13; and
the antigen-binding domain specifically binding to CLDN18.2 comprises the following variable region amino acid sequences or variants thereof:
(i) a heavy chain variable region amino acid sequence set forth in SEQ ID NO: 6, and
(ii) a light chain variable region amino acid sequence set forth in SEQ ID NO: 3; or
(14) the antigen-binding domain specifically binding to CD3 comprises the following variable region amino acid sequences or variants thereof:
(i) a heavy chain variable region amino acid sequence set forth in SEQ ID NO: 14, and
(ii) a light chain variable region amino acid sequence set forth in SEQ ID NO: 15; and
the antigen-binding domain specifically binding to CLDN18.2 comprises the following variable region amino acid sequences or variants thereof:
(i) a heavy chain variable region amino acid sequence set forth in SEQ ID NO: 6, and
(ii) a light chain variable region amino acid sequence set forth in SEQ ID NO: 3; or
(15) the antigen-binding domain specifically binding to CD3 comprises the following variable region amino acid sequences or variants thereof:
(i) a heavy chain variable region amino acid sequence set forth in SEQ ID NO: 16, and
(ii) a light chain variable region amino acid sequence set forth in SEQ ID NO: 17; and
the antigen-binding domain specifically binding to CLDN18.2 comprises the following variable region amino acid sequences or variants thereof:
(i) a heavy chain variable region amino acid sequence set forth in SEQ ID NO: 6, and
(ii) a light chain variable region amino acid sequence set forth in SEQ ID NO: 3;
wherein the variants differ from the variable region amino acid sequences by 3, 2 or 1 amino acids or have at least 80%, 85%, 90%, 91%, 92%, 93%, 94%, 95%, 96%, 97%, 98% or 99% identity thereto.

8. The bispecific antibody according to claim 1,
wherein the antigen-binding domain specifically binding to CLDN18.2 is in the form of an Fab fragment and the antigen-binding domain specifically binding to CD3 is in the form of an scFv, preferably, the bispecific antibody comprising:
a) a first monomer, comprising a first heavy chain,
wherein the first heavy chain comprises:
1) a first heavy chain variable region;
2) a first heavy chain constant region, comprising a first CH1 domain and a first Fc domain (wherein, preferably the first CH1 domain and the first Fc domain are linked by hinge region 1, and preferably the first Fc domain is selected from an IgG1 subtype, an IgG2 subtype, an IgG3 subtype and an IgG4 subtype);
3) an antigen-binding domain specifically binding to CD3 (preferably human CD3), wherein a heavy chain variable region and a light chain variable region in the antigen-binding domain are linked by a linker peptide and positions of the two are interchangeable, wherein the antigen-binding domain specifically binding to CD3 is as defined in claim 1, wherein
i) the antigen-binding domain is linked to a C-terminus of the first CH1 domain and an N-terminus of the first Fc domain by a linker peptide, preferably by a linker peptide and a hinge region (e.g., hinge region 3), and preferably the antigen-binding domain is linked to the first Fc domain by a linker peptide and a hinge region (e.g., hinge region 2), or
ii) the antigen-binding domain is linked to a C-terminus of the first Fc domain by a linker peptide, preferably by a linker peptide and/or a hinge region (e.g., hinge region 1, 2 or 3), or
iii) the antigen-binding domain is linked to an N-terminus of the first heavy chain variable region by a linker peptide, preferably by a linker peptide and/or a hinge region (e.g., hinge region 1, 2 or 3);
b) a second monomer, comprising a second heavy chain,
wherein the second heavy chain comprises: a second heavy chain variable region and a second heavy chain constant region, the second heavy chain constant region comprising a second CH1 domain and a second Fc domain (preferably the second CH1 domain and the second Fc domain are linked by a hinge region (e.g., hinge region 1), and preferably the second Fc domain is selected from an IgG1 subtype, an IgG2 subtype, an IgG3 subtype and an IgG4 subtype),
c) a first light chain assembled with the first heavy chain and comprising a first light chain variable region and a first light chain constant region;
d) a second light chain assembled with the second heavy chain and comprising a second light chain variable region and a second light chain constant region;
wherein the first light chain constant region is identical to or different from the second light chain constant regions, the first CH1 domain is identical to or different from the second CH1 domain, and the first Fc domain is identical to or different from the second Fc domain;
wherein the first heavy chain variable region and the first light chain variable region form a first antigen-binding domain, the second heavy chain variable region and the second light chain variable region form a second antigen-binding domain, and a sequence of the first antigen-binding domain and the second antigen-binding domain is a sequence of the antigen-binding domain specifically binding to CLDN18.2 as defined in claim 1, and the first antigen-binding domain and the second antigen-binding domain have identical or different sequences.

9. The bispecific antibody according to claim 1,
wherein the antigen-binding domain specifically binding to CLDN18.2 is in the form of an Fab fragment and the antigen-binding domain specifically binding to CD3 is in the form of an scFv, preferably, the bispecific antibody comprising:
a) a first monomer, comprising a heavy chain,
wherein the heavy chain comprises:
1) a first heavy chain variable region;
2) a heavy chain constant region comprising a CH1 domain and a first Fc domain (preferably the CH1 domain and the first Fc domain are linked by a hinge region (e.g., hinge region 1), and preferably the first Fc domain is selected from an IgG1 subtype, an IgG2 subtype, an IgG3 subtype and an IgG4 subtype);
b) a second monomer, comprising an antigen-binding domain specifically binding to CD3 (preferably human CD3), wherein the antigen-binding domain is linked to an N-terminus of a second Fc domain (preferably the second Fc domain is selected from an IgG1 subtype, an IgG2 subtype, an IgG3 subtype and an IgG4 subtype) by a linker peptide (preferably by a linker peptide and/or a hinge region (e.g., hinge region 1, 2 or 3)), wherein a heavy chain variable region and a light chain variable region in the antigen-binding domain specifically binding to CD3 are linked by a linker peptide and positions of the two are interchangeable, wherein the antigen-binding domain specifically binding to CD3 is as defined in claim 1,
c) a light chain assembled with the heavy chain and comprising a first light chain variable region and a light chain constant region;
wherein the first heavy chain variable region and the first light chain variable region form the antigen-binding domain specifically binding to CLDN18.2 as defined in claim 1.

10. The bispecific antibody according to claim 9, wherein the antigen-binding domain specifically binding to CLDN18.2 is selected from the group consisting of:
1) an antigen-binding domain specifically binding to CLDN18.2 comprising the following CDRs (or variants thereof) or variable regions (or variants thereof):
(i) CDRH1, CDRH2 and CDRH3 comprised in a heavy chain variable region set forth in SEQ ID NO: 5, or a heavy chain variable region set forth in SEQ ID NO: 5, and
(ii) CDRL1, CDRL2 and CDRL3 comprised in a light chain variable region set forth in SEQ ID NO: 2, or a light chain variable region set forth in SEQ ID NO: 2; or
2) an antigen-binding domain specifically binding to CLDN18.2 comprising the following CDRs (or variants thereof) or variable regions (or variants thereof):
(i) CDRH1, CDRH2 and CDRH3 comprised in a heavy chain variable region set forth in SEQ ID NO: 6, or a heavy chain variable region set forth in SEQ ID NO: 6, and
(ii) CDRL1, CDRL2 and CDRL3 comprised in a light chain variable region set forth in SEQ ID NO: 3, or a light chain variable region set forth in SEQ ID NO: 3,
wherein the variants differ from the CDRs or the variable regions by 3, 2 or 1 amino acids or have at least 80%, 85%, 90%, 91%, 92%, 93%, 94%, 95%, 96%, 97%, 98% or 99% identity thereto.

11. The bispecific antibody according to claim 8,
wherein the light chain constant region CL has a sequence set forth in SEQ ID NO: 18, 98, 99 or 100, and the CH1 domain has a sequence set forth in SEQ ID NO: 19, 101, 102 or 103, or
wherein the first Fc domain or the second Fc domain has identical or different sequence, preferably selected from SEQ ID NO: 20, 21, 22, 23, 24, 25 or 26, or
wherein the hinge region 1 has a sequence set forth in SEQ ID NO: 27, the hinge region 2 has a sequence set forth in SEQ ID NO: 28, and/or the hinge region 3 has a sequence set forth in SEQ ID NO: 29, or
wherein the linker peptide has a sequence selected from sequences set forth in SEQ ID NOs: 8, 30, 31 and 32.

12. The bispecific antibody according to claim 9,
wherein the light chain constant region CL has a sequence set forth in SEQ ID NO: 18, 98, 99 or 100, and the CH1 domain has a sequence set forth in SEQ ID NO: 19, 101, 102 or 103, or
wherein the first Fc domain or the second Fc domain has identical or different sequence, preferably selected from SEQ ID NO: 20, 21, 22, 23, 24, 25 or 26, or
wherein the hinge region 1 has a sequence set forth in SEQ ID NO: 27, the hinge region 2 has a sequence set forth in SEQ ID NO: 28, and/or the hinge region 3 has a sequence set forth in SEQ ID NO: 29, or
wherein the linker peptide has a sequence selected from sequences set forth in SEQ ID NOs: 8, 30, 31 and 32.

13. (canceled)

14. (canceled)

15. A nucleic acid composition, comprising a nucleic acid sequence encoding the bispecific antibody according to claim 1.

16. The nucleic acid composition according to claim 15, which is contained in an expression vector or a host cell.

17. (canceled)

18. A material which is selected from the group consisting of the followings:
(1) a pharmaceutical composition, comprising the bispecific antibody according to claim 1 and a pharmaceutical carrier, and optionally, a drug (e.g., a small molecule drug or a macromolecular drug) for the treatment of cancer (e.g., gastric cancer, pancreatic cancer, ovarian cancer, esophageal cancer or non-small cell lung cancer);
a kit, comprising the bispecific antibody according to claim 1, and optionally, a drug (e.g., a small molecule drug or a macromolecular drug) for the treatment of cancer (e.g., gastric cancer, pancreatic cancer, ovarian cancer, esophageal cancer or non-small cell lung cancer), and
a bispecific antibody conjugate, comprising the bispecific antibody according to claim 1 and a further substance, wherein the further substance is selected from one or more of a therapeutic agent, a prodrug, a peptide, a protein, an enzyme, a virus, a lipid, a biological response modifier, a pharmaceutical agent and PEG, and preferably the therapeutic agent comprises a detectable label, e.g., a radioactive label, an immunomodulator, a hormone, an enzyme, an oligonucleotide, a photoactive therapeutic or diagnostic agent, a cytotoxic agent such as a drug or a toxin, an ultrasound enhancing agent and a nonradioactive label.

19. (canceled)

20. (canceled)

21. A method for treating cancer, comprising administering to a subject a therapeutically effective amount of the bispecific antibody according to claim 1,
wherein the cancer is, for example, gastric cancer, pancreatic cancer, ovarian cancer, esophageal cancer or non-small cell lung cancer.

22. A bispecific antibody conjugate, comprising the bispecific antibody according to claim 1 and a further substance, wherein the further substance is selected from one or more of a therapeutic agent, a prodrug, a peptide, a protein, an enzyme, a virus, a lipid, a biological response modifier, a pharmaceutical agent and PEG, and preferably the therapeutic agent comprises a detectable label, e.g., a radioactive label, an immunomodulator, a hormone, an enzyme, an oligonucleotide, a photoactive therapeutic or diagnostic agent, a cytotoxic agent such as a drug or a toxin, an ultrasound enhancing agent and a nonradioactive label.

23. A nucleic acid composition, comprising:
a) a first expression vector comprising a first nucleic acid encoding the antigen-binding domain specifically binding to CD3 as defined in claim 5; and
b) a second expression vector comprising a second nucleic acid encoding the antigen-binding domain specifically binding to CLDN18.2 as defined in claim 5.

24. A nucleic acid composition, comprising:
a) a first expression vector comprising a first nucleic acid encoding the antigen-binding domain specifically binding to CD3 as defined in claim 6; and
b) a second expression vector comprising a second nucleic acid encoding the antigen-binding domain specifically binding to CLDN18.2 as defined in claim 6.

25. A nucleic acid composition, comprising:
a) a first expression vector comprising a first nucleic acid encoding the antigen-binding domain specifically binding to CD3 as defined in claim 7; and
b) a second expression vector comprising a second nucleic acid encoding the antigen-binding domain specifically binding to CLDN18.2 as defined in claim 7.
